(19) United States
(12) Patent Application Publication
(10) Pub. No.: US 2024/0307559 A1
(43) Pub. Date: Sep. 19, 2024

(54) KCNV2 GENE THERAPY

(71) Applicant: MeiraGTx UK II Limited, London (GB)

(72) Inventor: Anastasios Georgiadis, London (GB)

(21) Appl. No.: 18/578,623

(22) PCT Filed: Jul. 13, 2022
(86) PCT No.: PCT/IB2022/056457

§ 371 Date: Jan. 11, 2024

Related U.S. Application Data
(60) Provisional application No. 63/221,879, filed on Jul. 14, 2021.

(51) Int. Cl. A61K 48/00; A61K 38/17; C07K 14/705; C12N 15/86

(52) U.S. Cl. CPC A61K 48/0058; A61K 38/177; C07K 14/705; C12N 15/86; C12N 2750/14143; C12N 2830/008

(57) ABSTRACT

Provided herein are expression constructs, viral genomes, and vectors for the expression of Kv8.2, as well as pharmaceutical compositions comprising the vectors disclosed herein. Also provided are methods of using the expression constructs and vectors disclosed herein, including methods of treating a retinal disease in a subject in need thereof, wherein the retinal disease is associated with one or more mutations in the KCNV2 gene, the method comprising administering to the subject a vector disclosed herein.

### FIELD OF THE DISCLOSURE

The present disclosure relates generally to the field of molecular biology and medicine. More particularly, the invention provides compositions and methods for gene therapy for the treatment of retinal diseases.

### BACKGROUND

Kv8.2 is a voltage gated potassium channel subunit encoded by the KCNV2 gene. The KCNV2 gene is located on chromosome 9p24.2 and consists of 2 exons encoding a 545-amino acid protein. The protein is expressed in the retina in rod and cone photoreceptor inner segments (ellipsoid and myoid regions) and is absent in outer segments in human, mouse, and macaque. Kv8.2 interacts with other potassium subunits, such as Kv2.1, which is expressed in rods and cone inner segments, and with Kv2.2, which is expressed in cones but not rods in humans. Kv8.2 further interacts with Kv2 channels to alter their biophysical properties.

Kv8.2 is the only potassium channel subunit that has, thus far, been implicated in human disease. Variants/mutations of Kv8.2 cause a severe inherited photoreceptor dystrophy known as “cone-dystrophy with supernormal rod response” (CDSSR). Symptoms of CDSSR include reduced visual acuity, color vision defects, and altered electroretinogram responses, including elevated b-wave amplitudes.

Accordingly, novel therapies for the treatment of retinal diseases associated with KCNV2 mutations (including, but not limited to CDSSR) are urgently needed.

### SUMMARY OF THE DISCLOSURE

In one aspect the disclosure provides an expression construct comprising:
(a) a promotor sequence that confers expression in photoreceptor cells, and
(b) a nucleic acid sequence encoding Kv8.2;
wherein the nucleic acid sequence is operably linked to the promotor.

In embodiments, the promotor sequence is a CAG or rhodopsin kinase (RK) promotor sequence. In embodiments, the promotor sequence comprises a sequence that is at least 90% identical to SEQ ID: NO:8. In embodiments, the promotor sequence comprises a sequence of SEQ ID: NO:8. In other embodiments, the promotor sequence comprises a sequence that is at least 90% identical to SEQ ID: NO:7. In embodiments, the promotor sequence comprises a sequence of SEQ ID: NO:7.

In embodiments, the expression construct further comprises a post transcriptional regulatory element. In embodiments, the expression construct further comprises a woodchuck hepatitis virus post transcriptional regulatory element (WPRE). In embodiments, the WPRE comprises a sequence that is at least 90% identical to SEQ ID NO:11 or comprises the sequence of SEQ ID NO:11.

In embodiments, the nucleic acid sequence encoding the Kv8.2 is a coding sequence (cds) from a WT KCNV2 gene. In embodiments, the nucleic acid sequence encoding the Kv8.2 comprises a sequence that is at least 90% identical to SEQ ID NO:9. In embodiments, the nucleic acid sequence encoding the Kv8.2 comprises a sequence comprising SEQ ID NO:9.

In embodiments, the nucleic acid sequence encoding the Kv8.2 is a codon-optimized KCNV2 gene sequence. In embodiments, the nucleic acid sequence encoding the Kv8.2 comprises a sequence that is at least 90% identical to SEQ ID NO:10. In embodiments, the nucleic acid sequence encoding the Kv8.2 comprises a sequence comprising SEQ ID NO: 10.

In some embodiments, the nucleic acid sequence encoding the Kv8.2 encodes a protein comprising a sequence that is at least 90% identical to SEQ ID NO:13. In some embodiments, the nucleic acid sequence encoding the Kv8.2 encodes a protein comprising SEQ ID NO: 13.

In embodiments, the expression construct further comprises bovine growth hormone polyadenylation (BGH-polyA) signal. In embodiments, the polyadenylation signal comprises a sequence that is at least 90% identical to SEQ ID NO:12. In embodiments, the polyadenylation signal comprises SEQ ID NO:12.

In embodiments, expression construct comprises a sequence that is at least 90% identical to a sequence selected from the group consisting of SEQ ID NOS: 1-4. In embodiments, the expression construct comprises a sequence selected from the group consisting of SEQ ID NOS: 1-4.

In another aspect the disclosure provides a vector comprising an expression construct disclosed herein. In embodiments, the vector is a viral vector. In embodiments, vector is an adeno-associated virus (AAV) vector. In embodiments, vector comprises a genome derived from AAV serotype AAV2. In embodiments, the vector comprises a capsid derived from AAV7m8. In embodiments, the vector comprises a capsid derived from AAV5.

In another aspect, the disclosure provides a pharmaceutical composition comprising a vector disclosed herein and pharmaceutically acceptable carrier.

In another aspect, the disclosure provides a method for treating a retinal disease in a subject in need thereof, wherein the retinal disease is associated with one or more mutations in the KCNV2 gene, the method comprising administering to the subject a vector or a pharmaceutical composition disclosed herein. In embodiments, the retinal disease is cone-dystrophy with supernormal rod response (CDSSR).

In another aspect, the disclosure provides a method of increasing expression of KCNV2 in a subject in need thereof, the method comprising administering to the subject a vector or a pharmaceutical composition disclosed herein.

In another aspect, the disclosure provides a method of increasing Kv8.2 levels in a photoreceptor in a subject in need thereof, the method comprising administering to the subject a vector or a pharmaceutical composition disclosed herein.

In embodiments, the vector or the pharmaceutical composition is administered by intraocular injection. In embodiments of the disclosed methods, the vector or the pharmaceutical composition is injected into the central retina of the subject.

### BRIEF DESCRIPTION OF THE DRAWINGS

FIG. 1 shows a schematic of expression constructs: pCAG-KCNV2 WT, pCAG-KCNV2 Opti, pRT-KCNV2 WT, and pRT-KCNV2 Opti.

FIG. 2 illustrates the mRNA levels for KCNV2 WT and KCNV2 Opti in HEK293 cells relative to mRNA levels for KCNV2 WT and KCNV2 Opti in ARPE19 cells, as determined by qPCR (48 hours post transfection).

FIG. 3 shows Kv8.2 immunofluorescence of ARPE19 cells transfected with pCAG-GFP (top row), pCAG-KCNV2 Opti (middle row), and pCAG-KCNV2 WT (bottom row). Scale bar=10 μm.

FIGS. 4A and 4B show data from HEK293 cells analyzed by FACS. FIG. 4A. FACS data showing mean fluorescence intensity (MFI) from three independent experiments for HEK293 cells transfected with pCAG-KCNV2 WT and pCAG-KCNV2 Opti expression constructs, respectively. FIG. 4B. FACS data showing the percent of Kv8.2-Alexa 488 positive cell populations in non-transfected control vs. cells transfected with pCAG-KCNV2 WT or pCAG-KCNV2 Opti expression constructs, respectively. There was no significant difference in % Kv8.2 positive cells between codon optimized (Opti) and wild-type (WT) vectors.

FIG. 5A and FIG. 5B show transduction efficiency in transduced ARPE19 cells. FIG. 5A shows Kv8.2 fluorescent intensity in Kv8.2 immunolabeled ARPE19 cells transfected with the indicated AAV5 vectors at 2 multiplicities of infection (MOIs) (average integrated density per cell). FIG. 5B shows the percent of DAPI positive ARPE19 cells that were Kv8.2 positive after transduction with the indicated AAV5 vectors at 2MOIs.

FIG. 6 shows retinal organoid morphology. Live, brightfield imaging of whole retinal organoids. Typical morphology of WT (top row) and KCNV2 KO retinal organoids at day 140 when transduction occurred. Retinal organoids are laminated, have photoreceptor outer segments (‘brush borders’) and occasional clusters of retinal pigment epithelium (RPE). No gross morphological differences were observed between WT and knockout (KO) retinal organoids.

FIG. 7 shows transgenic Kv8.2 expression in KCNV2 KO retinal organoids (K28D5). Confocal tile scan analysis three weeks post transduction with AAV7m8. Signal is detected in the outermost photoreceptor cell layer. Scale bar=100 μm and 10 μm.

FIG. 8 shows AAV7m8 transduction of inner retinal cells. Transduced retinal cryosection co-stained with Kv8.2 and rod bipolar cell marker PKCa. WT organoids contained several Kv8.2 positive inner retinal cells (arrows) in the inner nuclear layer (INL) (separated from outer nuclear layer (ONL) by dashed line) in addition to the ONL, whereas the majority of Kv8.2 positive transduced cells were in the outer nuclear layer. pRK-KCNV2 vectors produced little detectable Kv8.2 protein (lower panel), although some Kv8.2 positive photoreceptors cells could be seen in the ONL of AAV7m8 RK-KCVN2 Opti condition (*). Scale bar=10 μm.

FIG. 9 shows AAV transduction of RPE cells. In addition to photoreceptors, RPE cells are present in organoids. RPE are present in clusters (arrow) rather than a planar sheet adjacent to the photoreceptor outer segments as seen in vivo. RPE are polarized and express CRALBP at their apical surface (A), nuclei are located basally (B). AAV efficiently transduced RPE cells in retinal organoids high levels of Kv8.2 are detected throughout the cytoplasm. In contrast, AAV RK-KCNV2 did not produce detectable levels of Kv8.2 expression in RPE cells.

FIG. 10 shows the transduction of Muller Glia cells with AAV5 pCAG-KCNV2-Opti, AAV5 pRK-KCNV2-Opti, AAV7m9 pCAG-KCNV2-Opti, and AAV7m9 pRK-KCNV2-Opti, respectively. CRALBP stains Muller Glia cells, which span the neural retina and form the outer limiting membrane via tight junctions with rod and cone cells. Despite their close proximity to photoreceptor cells, Muller Glia did not co-stain for Kv8.2.

FIG. 11 shows that transgenic Kv8.2 expression is localized to the plasma membrane and photoreceptor inner segments. Clone K28 (differentiation 5) transduced with 7m8 AAV vectors with both codon optimized and WT KCNV2 vectors driven by the CAG promoter (CAG KCNV2-WT and 7m8 CAG KCVN2 Opti) stained with rhodopsin and Kv8.2. Nuclei are counterstained with DAPI.

FIG. 12 shows Kv8.2 co-localization with Kv.2.1 at the photoreceptor inner segment. High magnification confocal microscopy of WT vs. transduced KCNV2 KO organoids transduced with AAV5 CAG-WT or AAV5 CAG-Opti vectors. Potassium channel Kv2.1 localizes to the plasma membrane of globular inner segment structures, vector derived Kv8.2 is co-expressed in the inner segment in a similar expression pattern to the WT.

FIGS. 13A, 13B, and 13C show TUNEL staining in WT, control, and transduced retinal organoids. FIG. 13A Whole confocal tile scans (40× magnification) of WT retinal organoid cryosections stained with DAPI and terminal deoxynucleotidyl transferase dUTP nick end labeling (TUNEL), an indicator of apoptosis. TUNEL reactivity was scarce to non-existent in the retinal cell layers (INL and ONL) but could be detected in the center of the organoid (dashed region) and in areas of non-retinal tissue. FIG. 13B Qualitatively, there was no increase in TUNEL reactivity in KCNV2 KO organoids relative to WT and no increase in KO organoids transduced with AAV 7m8. FIG. 13C. Qualitatively there was no increase in TUNEL reactivity in KCNV2 KO organoids relative to WT and no increase in KO organoids transduced with AAV5 vectors comprising WT or codon optimized KCNV2.

FIG. 14 shows cone cell numbers in AAV transduced retinal organoids. LM opsin staining was used to determine the average number of cones per 100 μm in WT, KCNV2 KO, and organoids transduced with the indicated vectors. Each point represents one transduced organoid for which cones were counted from one 7 μm retinal cryosection. The whole organoid was imaged at 40× magnification and cones per μm counted (between 34 and 483 counted per organoid). All organoids had a good distribution of cone cells. Non-transduced KCNV2 KO organoids had significantly more cones than WT non-edited controls (p=0.004, unpaired t-tests). AAV transduction (all vectors grouped) did not reduce cone numbers relative to WT (p=0.2) but did reduce cone numbers relative to non-transduced p=0.02. Error bars=Stdev.

FIGS. 15A and 15B show relative mRNA levels in transduced retinal organoids, determined by qPCR. FIG. 15A KCNV2 expression in KCNV2 KO clones K5, K12, and K28 transduced With the different AAV vectors containing either photoreceptor specific RK or constitutive CAG promoter driving the expression of two different versions of the KCNV2 gene: codon optimized or WT. Untreated KO KCNV2 clones and WT isogenic control, 15, are included for comparison. Graph shows expression of codon optimized KCNV2, 21 days post transduction with different AAV vectors, AAV5-CAG-KCNV2opti, AAV7m8-CAG-KCNV2opti, AAV5-RK-KCNV2opti, or AAV7m8-RK-KCNV2-Opti. Results are expressed as fold change in KCNV2 mRNA expression relative to the lowest expressing sample (AAV5 CAG-KCNV2-Opti). FIG. 15B A graph showing the expression of WT KCNV2, 21 days post transduction with indicated AAV vectors. Results are expressed as fold change in WT KCNV2 mRNA relative to age matched un-transduced control from the same KO clone.

FIGS. 16A and 16B show quantification of retinal organoid immunofluorescence. FIG. 16A shows total Kv8.2 fluorescence in the outer nuclear layer (ONL). Bars=mean fluorescence in transduced organoids normalized to measured area and expressed as a % fluorescence in WT control organoid. Tile scan images were acquired to obtain fluorescence measurements over the whole length of the ONL. Dotted line represents the mean ‘background’ signal in non-transduced KO control organoids. Each dot represents one organoid. n=3-4 organoids from independent experiments. Error bars=+/−SEM. There was a significant difference in total fluorescence between CAG and RK promoters in both 7m8 and AAV5 capsids (p=0.031 and 0.028 respectively, 2 tailed, paired student's t test). There was no significant difference between WT and Opti in vectors with CAG or RK promoters despite a trend towards increased fluorescence intensity in codon optimized (Opti) vectors.

FIG. 16B shows representative immunofluorescence in the photoreceptors layer of transduced KCNV2 KO, WT, and AAV 7m8 Kv8.2 transduced organoids. Kv8.2 and potassium channel subunit Kv.2.1, cone Arrestin (Arr3) and nuclei are stained with DAPI. Scale bar=10 μm.

FIG. 17 shows relative co-localization of Kv8.2 and Kv2.1 in transduced organoids. Organoid cryosections were co-stained with Kv.2.1 and Kv8.2 and the total co-localizing area was measured on thresholded images in FIJI and normalized to the length of the retina assayed per organoid. Results are expressed as a fold change relative to non-transduced control. Results were analyzed by one way ANOVA and Dunnett's multiple comparison test. *p=0.02, **p=0.002.

FIGS. 18A, 18B, and 18C show proximity ligation assay (PLA) signal specificity in transduced organoids. FIG. 18A. PLA signal following Kv2.1 and Kv8.2 co-stain (dots) was abundant in retinal organoids at the outer periphery where photoreceptor inner/outer segments are situated. FIG. 18B PLA signal was largely absent in the ONL (photoreceptor layer) of KCNV2 KO organoids. FIG. 18C Quantification of PLA puncta (imaged J) normalized to area of measurement (n=3 regions of interest (ROIs)) per organoid. There was a significant reduction in PLA signal in KCNV2 KO photoreceptors relative to WT (clones K28 and K12) (p<0.03). Error bars=SEM.

FIG. 19 shows the PLA signal in AAV5 and AAV7m8 transduced photoreceptors. 63× maximum intensity projections from 7 μm organoid cryosections. The photoreceptor layer (ONL) has a distinctive compact structure visible in the DAPI channel above the outer plexiform layer, which does not have any nuclei. The PLA signal (dots) signifies Kv.2.1/Kv8.2 protein-protein interactions. The PLA signal was concentrated at the apical edge of the ONL, in the region of the photoreceptor inner segments (IS). Transduced organoids had a higher PLA signal density than non-transduced KCNV2 KO organoids derived from IPSC clone K12.

FIGS. 20A and 20B shows quantification of PLA puncta in two transduced retinal organoid clones (clone 12 and clone 28 respectively). Bars represent the average number of PLA puncta in the ONL per field of view (approximately 32 per 100-150 photoreceptors, 10-600 puncta counted per field of view) normalized to the measured area. Error bars=SEM).

### DETAILED DESCRIPTION

Provided herein are expression constructs, viral genomes, and vectors for the expression of Potassium Voltage-Gated Channel Modifier Subfamily V Member 2 (Kv8.2), as well as methods of using the expression constructs, viral genomes, and vectors for treating a retinal disease associated with one or more mutations in the KCNV2 gene.

### Kv8.2

Kv8.2 is a voltage gated potassium channel subunit. The KCNV2 gene is located on chromosome 9p24.2 and comprises 2 exons encoding the 545-amino acid Kv8.2 protein. Kv8.2 cannot form functional homomeric channels but interact with other potassium channel subunits, Kv2.1 and Kv2.2 to alter their biophysical properties. Kv8.2 is the only silent subunit that has thus far been implicated in human disease. Variants/mutations cause a severe inherited photoreceptor dystrophy known as “cone-dystrophy with supernormal rod response” (CDSSR).

KCNV2 (Kv8.2) is expressed in the retina in rod and cone photoreceptor inner segments (ellipsoid and myoid regions) and is absent in outer segments in human, mouse, and macaque. Kv8.2 interacts with Kv2.1, which is expressed in rods and cone inner segments, and with Kv2.2, which is expressed in cones but not rods in humans.

A KCNV2 (Kv8.2) homozygous knock-out (KO) mice show many similarities to the human disorder, including an electroretinogram (ERG) with reduced a-wave and an elevated b-wave response to bright light stimulation. KCNV2 KO mice exhibit a reduction in cone cell numbers (80% of WT), an increase in TUNEL positive cells throughout the retina (at 1, 3 and 6 months old) and an overall thinning of the outer nuclear layer (ONL 60% of the WT at 6 months old).

The correct sub-cellular localization of many important photoreceptor proteins has been demonstrated previously (e.g. Rhodopsin, RetGC, ABCA4 are located in the rod outer segments; Bassoon, Ribeye are located at the synaptic terminal). The presence of the potassium channel subunits Kv8.2, Kv2.1, and Kv2.2 in human embryonic stem cells (HESC) or induced pluripotent stem cells (IPSC) derived human retinal organoids has not yet been investigated in any publications although the presence of the KCNV2 transcript has been detected in human retinal organoids by single cell RNA seq. Documented species-specific differences in the function of Kv8.2 and its binding partners (e.g., the absence of Kv2.2 in mouse retina) make the use of human cell models important to the development of a potential KCNV2 AAV gene therapy.

Mutations in KCNV2 can cause retinal disease including photoreceptor dystrophies, such as cone dystrophy with supernormal rod response (CDSSR). The diagnosis of such diseases is established by electrophysiological evaluation; functional results depend on the stage of the disease and the age of the individual. For example, CDSSR is associated with an electroretinogram (ERG) with reduced a-wave and an elevated b-wave response to bright light stimulation. A diagnosis of cone dystrophies may be supported by a demonstration of reduced cone cell numbers (˜80% of normal). For example, retinas having abnormal KCNV2 expression may have increased TUNEL positive cells and an overall thinning of the outer nuclear layer (ONL, 60% of normal).

### Expression Constructs

In one aspect, provided is an expression construct comprising: (a) a promotor sequence that confers expression in photoreceptor cells, and (b) a nucleic acid sequence encoding Potassium Voltage-Gated Channel Modifier Subfamily V Member 2 (Kv8.2); wherein the nucleic acid sequence is operably linked to the promoter. As used herein, “operably linked” refers to both expression control sequences (e.g., promoters) that are contiguous with the coding sequences for Kv8.2 and expression control sequences that act in trans or at a distance to control the expression of Kv8.2. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein processing and/or secretion.

A great number of expression control sequences, e.g., native, constitutive, inducible and/or tissue-specific, are known in the art and may be utilized to drive expression of the gene, depending upon the type of expression desired. For eukaryotic cells, expression control sequences typically include a promoter, an enhancer, and a polyadenylation sequence which may include splice donor and acceptor sites. The polyadenylation (poly A) sequence generally is inserted following the sequence encoding Kv8.2 and before the 3′ ITR sequence. Another regulatory component of the rAAV useful in the methods disclosed herein is an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the sequence encoding Kv8.2 in the rAAV vector.

In one embodiment, the promotor sequence comprises a rhodopsin kinase (RK) promoter sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7. In one embodiment, the promotor sequence comprises SEQ ID NO:7.

In one embodiment, the promotor sequence comprises a synthetic cytomegalovirus-derived promotor sequence (CAG). In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8. In one embodiment, the promotor sequence comprises SEQ ID NO:8.

In some embodiments, the promoter is specific to photoreceptor cells, that is, the promoter has activity in photoreceptor cells, but has reduced or no activity in other cell types.

In one embodiment, the nucleic acid sequence encoding the Kv8.2 is a coding sequence from a WT KCNV2 gene. In some embodiments, the nucleic acid sequence encoding the Kv8.2 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:9. In one embodiment, the nucleic acid sequence encoding the Kv8.2 comprises SEQ ID NO:9.

In one embodiment, the nucleic acid sequence encoding the Kv8.2 is a codon-optimized sequence. In some embodiments, the nucleic acid sequence encoding the Kv8.2 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In one embodiment, the nucleic acid sequence encoding the Kv8.2 comprises SEQ ID NO:10.

In some embodiments, the nucleic acid sequence encoding the Kv8.2 encodes a protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:13. In some embodiments, the nucleic acid sequence encoding the Kv8.2 encodes a protein comprising SEQ ID NO: 13.

In one embodiment, the expression construct comprises a post transcriptional regulatory element. In one embodiment, the expression construct comprises a woodchuck hepatitis virus post transcriptional regulatory element (WPRE). In some embodiments, the post transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 11. In one embodiment, the post transcriptional regulatory element comprises SEQ ID NO:11.

In one embodiment, the expression construct comprises a polyadenylation signal. In one embodiment, the expression construct comprises a bovine growth hormone polyadenylation (BGH-polyA) signal. In some embodiments, the polyadenylation signal comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12. In one embodiment, the polyadenylation signal comprises SEQ ID NO: 12.

### Vectors

In one aspect, provided are recombinant vectors and their use for the introduction of a transgene or an expression construct into a cell. In some embodiments, the recombinant vectors comprise recombinant DNA constructs that include additional DNA elements including DNA segments that provide for the replication of the DNA in a host cell and expression of the target gene in target cells at appropriate levels. The ordinarily skilled artisan appreciates that expression control sequences (promoters, enhancers, and the like) are selected based on their ability to promote expression of the target gene in the target cell. “Vector,” as used herein, means a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus derived sequences). A vector may also refer to a virion comprising a nucleic acid to be delivered into a host cell, either in vitro or in vivo. In some embodiments, a vector refers to a virion comprising a recombinant viral genome, wherein the recombinant viral genome comprises one or more ITRs and a transgene.

In one embodiment, the recombinant vector is a viral vector or a combination of multiple viral vectors.

In one aspect, provided is a vector comprising any of the expression constructs disclosed herein.

In one aspect, provided is a vector comprising a nucleic acid comprising (a) a promotor sequence that confers expression in photoreceptor cells, and (b) a nucleic acid sequence encoding Kv8.2, wherein the nucleic acid sequence encoding Kv8.2 is operably linked to the promoter.

In one embodiment, the promotor sequence comprises a rhodopsin kinase (RK) promoter sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7. In one embodiment, the promotor sequence comprises SEQ ID NO:7.

In one embodiment, the promotor sequence comprises a synthetic cytomegalovirus-derived promotor sequence (CAG). In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8. In one embodiment, the promotor sequence comprises SEQ ID NO:8.

In some embodiments, the promoter is specific to photoreceptor cells.

In one embodiment, the nucleic acid sequence encoding the Kv8.2 is a coding sequence from a WT KCNV2 gene. In some embodiments, the nucleic acid sequence encoding the Kv8.2 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:9. In one embodiment, the nucleic acid sequence encoding the Kv8.2 comprises SEQ ID NO:9.

In one embodiment, the nucleic acid sequence encoding the Kv8.2 is a codon-optimized sequence. In some embodiments, the nucleic acid sequence encoding the Kv8.2 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In one embodiment, the nucleic acid sequence encoding the Kv8.2 comprises SEQ ID NO:10.

In some embodiments, the nucleic acid sequence encoding the Kv8.2 encodes a protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:13. In some embodiments, the nucleic acid sequence encoding the Kv8.2 encodes a protein comprising SEQ ID NO: 13.

In one embodiment, the vector comprises a nucleic acid comprising a post transcriptional regulatory element. In one embodiment, the vector comprises a nucleic acid comprising a woodchuck hepatitis virus post transcriptional regulatory element (WPRE). In some embodiments, the post transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11. In one embodiment, the post transcriptional regulatory element comprises SEQ ID NO:11.

In one embodiment, the vector comprises a nucleic acid comprising a polyadenylation signal. In one embodiment, the vector comprises a nucleic acid comprising a bovine growth hormone polyadenylation (BGH-polyA) signal. In some embodiments, the polyadenylation signal comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12. In one embodiment, the polyadenylation signal comprises SEQ ID NO:12.

In one embodiment, the vector comprises a nucleic acid comprising one or more inverted terminal repeats (ITR). In one embodiment, the ITR sequence is derived from AAV serotype 2. In one embodiment, the 5′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:5. In one embodiment, the 5′ ITR sequence comprises SEQ ID NO:5. In one embodiment, the 3′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:6. In one embodiment, the 3′ ITR sequence comprises SEQ ID NO:6.

In some embodiments, the vector comprises a nucleic acid comprising a sequence selected from the group consisting of SEQ ID NOS: 1-4.

In one embodiment, provided is a vector comprising a nucleic acid comprising one or more of:
(a) promotor sequence comprising an RK promoter sequence;
(b) a nucleic acid sequence encoding Kv8.2, wherein the nucleic acid sequence encoding Kv8.2 is operably linked to the promoter;
(c) a WPRE;
(d) a BGH-polyA signal; and
(e) one or more ITRs. In some embodiments, the nucleic acid comprises two ITR sequences.

In one embodiment, provided is a vector comprising a nucleic acid comprising one or more of:
(a) promotor sequence comprising a CAG promoter sequence;
(b) a nucleic acid sequence encoding Kv8.2, wherein the nucleic acid sequence encoding Kv8.2 is operably linked to the promoter;
(c) a WPRE;
(d) a BGH-poly A signal; and
(e) one or more ITRs. In some embodiments, the nucleic acid comprises two ITR sequences.

In one embodiment, provided is a vector comprising a nucleic acid comprising one or more of:
(a) promotor sequence comprising an RK promoter sequence;
(b) a codon optimized nucleic acid sequence encoding Kv8.2, wherein the nucleic acid sequence encoding Kv8.2 is operably linked to the promoter;
(c) a WPRE;
(d) a BGH-polyA signal; and
(e) one or more ITRs. In some embodiments, the nucleic acid comprises two ITR sequences.

In one embodiment, provided is a vector comprising a nucleic acid comprising one or more of:
(a) promotor sequence comprising a CAG promoter sequence;
(b) a codon optimized nucleic acid sequence encoding Kv8.2, wherein the nucleic acid sequence encoding Kv8.2 is operably linked to the promoter;
(c) a WPRE;
(d) a BGH-polyA signal; and
(e) one or more ITRs. In some embodiments, the nucleic acid comprises two ITR sequences.

In one embodiment, provided is a vector comprising a nucleic acid comprising one or more of:
(a) a promotor sequence comprising a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12; and
(e) one or more ITRs. In some embodiments, the nucleic acid comprises two ITR sequences.

In one embodiment, provided is a vector comprising a nucleic acid comprising one or more of:
(a) a promotor sequence comprising a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12; and
(e) one or more ITRs. In some embodiments, the nucleic acid comprises two ITR sequences.

In one embodiment, provided is a vector comprising a nucleic acid comprising one or more of:
(a) a promotor sequence comprising a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:10;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12; and
(e) one or more ITRs. In some embodiments, the nucleic acid comprises two ITR sequences.

In one embodiment, provided is a vector comprising a nucleic acid comprising one or more of:
(a) a promotor sequence comprising a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:10;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12; and
(e) one or more ITRs. In some embodiments, the nucleic acid comprises two ITR sequences.

In one embodiment, provided is a vector comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding a Kv8.2 protein, wherein the Kv8.2 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:13, and wherein the nucleic acid sequence encoding the Kv8.2 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12; and
(e) one or more ITRs. In some embodiments, the nucleic acid comprises two ITR sequences.

In one embodiment, provided is a vector comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding a Kv8.2 protein, wherein the Kv8.2 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO: 13 and wherein the nucleic acid sequence encoding the Kv8.2 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:7;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:9;
(c) a post transcriptional regulatory element comprising SEQ ID NO:11;
(d) a polyadenylation signal comprising a sequence SEQ ID NO: 12; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:8;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:9;
(c) a post transcriptional regulatory element comprising SEQ ID NO:11;
(d) a polyadenylation signal comprising a sequence SEQ ID NO:12; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:7;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:10;
(c) a post transcriptional regulatory element comprising SEQ ID NO:11;
(d) a polyadenylation signal comprising a sequence SEQ ID NO: 12; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:8;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:10;
(c) a post transcriptional regulatory element comprising SEQ ID NO:11;
(d) a polyadenylation signal comprising a sequence SEQ ID NO:12; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:7;
(b) a nucleic acid sequence encoding a Kv8.2 protein, wherein the Kv8.2 protein comprises SEQ ID NO:13, and wherein the nucleic acid sequence encoding the Kv8.2 protein is operably linked to the promoter;
(c) a post transcriptional regulatory element comprising SEQ ID NO:11;
(d) a polyadenylation signal comprising a sequence SEQ ID NO: 12; and
(e) one or more ITRs. In some embodiments, the nucleic acid comprises two ITR sequences.

In one embodiment, provided is a vector comprising a nucleic acid comprising one or more of:
(a) a promotor sequence comprising SEQ ID NO:8;
(b) a nucleic acid sequence encoding a Kv8.2 protein, wherein the Kv8.2 protein comprises SEQ ID NO:13, and wherein the nucleic acid sequence encoding the Kv8.2 protein is operably linked to the promoter;
(c) a post transcriptional regulatory element comprising SEQ ID NO:11;
(d) a polyadenylation signal comprising a sequence SEQ ID NO: 12; and
(e) one or more ITRs. In some embodiments, the nucleic acid comprises two ITR sequences.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The Rep reading frame encodes four proteins of molecular weight 78 kD, 68 kD, 52 kD and 40 kD. These proteins function mainly in regulating AAV replication and rescue and integration of the AAV into a host cell's chromosomes. The Cap reading frame encodes three structural proteins of molecular weight 85 kD (VP 1), 72 kD (VP2) and 61 kD (VP3), which form the virion capsid. More than 80% of total proteins in AAV virion comprise VP3. Flanking the rep and cap open reading frames at the 5′ and 3′ ends are about 141 bp long ITRs. The ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, preferably the Rep and/or Cap genes, but retain functional flanking ITR sequences.

In some embodiments, the viral vector is an rAAV virion, which comprises an rAAV genome and one or more capsid proteins. In some embodiments, the rAAV genome comprises an expression cassette disclosed herein.

In some embodiments, the viral vector disclosed herein comprises a nucleic acid comprising AAV 5′ ITRs and 3′ ITRs located 5′ and 3′ to sequence encoding Kv8.2, respectively. However, in certain embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to the sequence encoding Kv8.2. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh. 10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/5 (i.e., an AAV containing AAV2 ITRs and AAV5 capsid proteins). In some embodiments, the pseudotyped AAV is AAV2/7m8 (i.e., an AAV containing AAV2 ITRs and AAV7m8 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In one aspect, provided is a viral genome comprising a nucleic acid comprising (a) a promotor sequence that confers expression in photoreceptor cells, and (b) a nucleic acid sequence encoding Kv8.2, wherein the nucleic acid sequence encoding Kv8.2 is operably linked to the promoter.

In one embodiment, the promotor sequence comprises a RK promoter sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7. In one embodiment, the promotor sequence comprises SEQ ID NO:7.

In one embodiment, the promotor sequence comprises a CAG promotor sequence. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8. In one embodiment, the promotor sequence comprises SEQ ID NO:8.

In some embodiments, the promoter is specific to photoreceptor cells.

In one embodiment, the nucleic acid sequence encoding the Kv8.2 is a coding sequence from a wild-type KCNV2gene. In some embodiments, the nucleic acid sequence encoding the Kv8.2 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:9. In one embodiment, the nucleic acid sequence encoding the RetGC comprises SEQ ID NO:9.

In one embodiment, the nucleic acid sequence encoding the Kv8.2 is a codon-optimized sequence. In some embodiments, the nucleic acid sequence encoding the Kv8.2 comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In one embodiment, the nucleic acid sequence encoding the RetGC comprises SEQ ID NO:10.

In some embodiments, the nucleic acid sequence encoding the Kv8.2 encodes a protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:13. In some embodiments, the nucleic acid sequence encoding the Kv8.2 encodes a protein comprising SEQ ID NO:13.

In one embodiment, the viral genome comprises a nucleic acid comprising a post transcriptional regulatory element. In one embodiment, the viral genome comprises a nucleic acid comprising a WPRE. In some embodiments, the post transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11. In one embodiment, the post transcriptional regulatory element comprises SEQ ID NO:11.

In one embodiment, the viral genome comprises a nucleic acid comprising a polyadenylation signal. In one embodiment, the viral genome comprises a nucleic acid comprising a BGH-polyA signal. In some embodiments, the polyadenylation signal comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12. In one embodiment, the polyadenylation signal comprises SEQ ID NO:12.

In one aspect, the viral genome comprises a nucleic acid comprising one or more inverted terminal repeats (ITR). In one embodiment, the ITR sequence is derived from AAV serotype 2. In one embodiment, the 5′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:5. In one embodiment, the 5′ ITR sequence comprises SEQ ID NO:5. In one embodiment, the 3′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:6. In one embodiment, the 3′ ITR sequence comprises SEQ ID NO:6.

In some embodiments, the viral genome comprises a nucleic acid comprising a sequence that is at least 80%, at least 85%, at least 90%, at least 91%, at least 92%, at least 93%, at least 94%, at least 95%, at least 96%, at least 97%, at least 98%, or at least 99% identical to any of the sequences of SEQ ID NOS: 1-4. In some embodiments, the viral genome comprises a nucleic acid comprising a sequence selected from the group consisting of SEQ ID NOS: 1-4.

In one embodiment, provided is a viral genome comprising a nucleic acid comprising one or more of:
(a) promotor sequence comprising an RK promoter sequence;
(b) a nucleic acid sequence encoding a Kv8.2, wherein the nucleic acid sequence encoding Kv8.2 is operably linked to the promoter;
(c) a WPRE;
(d) a BGH-polyA signal; and
(e) one or more ITRs. In some embodiments, the viral genome comprises two ITR sequences.

In one embodiment, provided is a viral genome comprising a nucleic acid comprising one or more of:
(a) promotor sequence comprising a CAG promoter sequence;
(b) a nucleic acid sequence encoding a Kv8.2, wherein the nucleic acid sequence encoding Kv8.2 is operably linked to the promoter;
(c) a WPRE;
(d) a BGH-polyA signal; and
(e) one or more ITRs. In some embodiments, the viral genome comprises two ITR sequences.

In one embodiment, provided is a viral genome comprising a nucleic acid comprising one or more of:
(a) promotor sequence comprising an RK promoter sequence;
(b) a nucleic acid sequence encoding a codon optimized Kv8.2, wherein the nucleic acid sequence encoding Kv8.2 is operably linked to the promoter;
(c) a WPRE;
(d) a BGH-polyA signal; and
(e) one or more ITRs. In some embodiments, the viral genome comprises two ITR sequences.

In one embodiment, provided is a viral genome comprising a nucleic acid comprising one or more of:
(a) promotor sequence comprising a CAG promoter sequence;
(b) a nucleic acid sequence encoding a codon optimized Kv8.2, wherein the nucleic acid sequence encoding Kv8.2 is operably linked to the promoter;
(c) a WPRE;
(d) a BGH-polyA signal; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:9;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding a codon optimized Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:10;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:12; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding a codon optimized Kv8.2, wherein the nucleic acid sequence encoding Kv8.2 is operably linked to the promoter and wherein the nucleic acid sequence encoding Kv8.2 comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:10;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12; and
(e) one or more ITRs. In some embodiments, the viral genome comprises two ITR sequences.

In one embodiment, provided is viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:7;
(b) a nucleic acid sequence encoding a Kv8.2 protein, wherein the Kv8.2 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO:13, and wherein the nucleic acid sequence encoding the Kv8.2 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:8;
(b) a nucleic acid sequence encoding a Kv8.2 protein, wherein the Kv8.2 protein comprises a sequence that is least 80%, at least 85%, at least 90%, at least 91%, at least 92%, at least 93%, at least 94%, at least 95%, at least 96%, at least 97%, at least 98%, or at least 99% identical to SEQ ID NO: 13 and wherein the nucleic acid sequence encoding the Kv8.2 protein is operably linked to the promoter;
(c) a post transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:7;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:9;
(c) a post transcriptional regulatory element comprising SEQ ID NO:11;
(d) a polyadenylation signal comprising a sequence SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:8;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:9;
(c) a post transcriptional regulatory element comprising SEQ ID NO:11;
(d) a polyadenylation signal comprising a sequence SEQ ID NO:12; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:7;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:10;
(c) a post transcriptional regulatory element comprising SEQ ID NO:11;
(d) a polyadenylation signal comprising a sequence SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:8;
(b) a nucleic acid sequence encoding Kv8.2, wherein the nucleic acid sequence encoding Kv8.2 is operably linked to the promoter and wherein nucleic acid sequence encoding Kv8.2 comprises SEQ ID NO:10;
(c) a post transcriptional regulatory element comprising SEQ ID NO:11;
(d) a polyadenylation signal comprising a sequence SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:7;
(b) a nucleic acid sequence encoding a Kv8.2 protein, wherein the Kv8.2 protein comprises SEQ ID NO:13, and wherein the nucleic acid sequence encoding the Kv8.2 protein is operably linked to the promoter;
(c) a post transcriptional regulatory element comprising SEQ ID NO:11;
(d) a polyadenylation signal comprising a sequence SEQ ID NO: 12; and
(e) one or more ITRs. In some embodiments, the viral genome comprises two ITR sequences.

In one embodiment, provided is a viral genome comprising a nucleic acid comprising one or more of:
(a) a promotor sequence comprising SEQ ID NO:8;
(b) a nucleic acid sequence encoding a Kv8.2 protein, wherein the Kv8.2 protein comprises SEQ ID NO:13, and wherein the nucleic acid sequence encoding the Kv8.2 protein is operably linked to the promoter;
(c) a post transcriptional regulatory element comprising SEQ ID NO:11;
(d) a polyadenylation signal comprising a sequence SEQ ID NO: 12; and
(e) one or more ITRs. In some embodiments, the viral genome comprises two ITR sequences.

Adenoviral (AV) vectors include, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Alternatively, other systems such as lentiviral vectors can be used. Lentiviral-based systems can transduce nondividing as well as dividing cells making them useful for applications targeting, for examples, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the expression cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells includes hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this invention are known to those with skill in nucleic acid manipulation and include genetic engineering, recombinant engineering, and synthetic techniques. See, e.g., Sambrook et al, and Ausubel et al., cited above; and International Patent Publication No. WO 95/13598. Further, methods suitable for producing a rAAV cassette in an adenoviral capsid have been described in U.S. Pat. Nos. 5,856,152 and 5,871,982.

Briefly, in order to package the rAAV genome into a rAAV virion, a host cell must contain sequences necessary to express AAV rep and AAV cap or functional fragments thereof as well as helper genes needed for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection and protoplast fusion. In one embodiment, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

The rep and cap sequences, along with their expression control sequences, may be supplied on a single vector, or each sequence may be supplied on its own vector. Preferably, the rep and cap sequences are supplied on the same vector. Alternatively, the rep and cap sequences may be supplied on a vector that contains other DNA sequences that are to be introduced into the host cells. Preferably, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this invention are conventional genetic engineering or recombinant engineering techniques such as those described in the references above. For example, the rAAV may be produced utilizing a triple transfection method using either the calcium phosphate method (Clontech) or Effectene reagent (Qiagen, Valencia, Calif.), according to manufacturer's instructions. See, also, Herzog et al, 1999, Nature Medic., 5(1):56-63, for the method used in the following examples, employing the plasmid with the transgene, CPA-RPE65, a helper plasmid containing AAV rep and cap, and a plasmid supplying adenovirus helper functions of E2A, E4Orf6 and VA. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions are then produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art, preferably on a separate plasmid. Thereafter, the recombinant AAV virion which directs expression of the transgene is isolated from the cell or cell culture in the absence of contaminating helper virus or WT AAV.

Expression of the KCNV2 gene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR (qPCR); and the level of protein expression may be monitored by Western blotting, immunohistochemistry, enzyme-linked immunosorbent assay (ELISA), radioimmunoassay (RIA) or by the specific methods detailed below in the Examples.

### Pharmaceutical Compositions

Provided herein are pharmaceutical composition comprising any of the vectors disclosed herein and a pharmaceutically acceptable excipient.

The recombinant AAV containing the gene encoding Kv8.2 is preferably assessed for contamination by conventional methods and then formulated into a pharmaceutical composition suitable for administration to a patient.

Such formulation involves the use of a pharmaceutically and/or physiologically acceptable vehicle or carrier, particularly one suitable for subretinal injection, such as buffered saline or other buffers, e.g., HEPES, to maintain pH at appropriate physiological levels

The vector of the invention can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilisers, buffers, antioxidants and/or other additives may be included, as required.

For delayed release, the vector may be included in a pharmaceutical composition which is formulated for slow release, such as in microcapsules formed from biocompatible polymers or in liposomal carrier systems according to methods known in the art.

If the virus is to be stored long-term, it may be frozen in the presence of glycerol.

### Methods of Treatment

Provided herein is a method of treating a retinal disease in a subject in need thereof, wherein the retinal disease is associated with one or more mutations in the KCNV2 gene, the method comprising administering to the subject a vector disclosed herein. Provided herein is a vector for use in a method of treating a retinal disease in a subject in need thereof, wherein the retinal disease is associated with one or more mutations in the KCNV2 gene. In some embodiments, the subject carries a mutation in the KCNV2.

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In some embodiments, the retinal disease is a cone-dystrophy. In one embodiment, the retinal disease is cone-dystrophy with supernormal rod response (CDSSR).

In one aspect, provided is a method comprising:
(a) determining whether a subject carries a mutation in the KCNV2 gene; and
(b) administering a pharmaceutical composition comprising a vector disclosed herein to the subject if the subject carries a mutation in the KCNV2 gene.

### Route and Methods of Administration

In some embodiments, the vectors or the pharmaceutical compositions disclosed herein are administered by intraocular injection. In some embodiments, the vectors or the pharmaceutical compositions disclosed herein are administered by direct retinal, subretinal, or intravitreal injection. In some embodiments, the vectors or the pharmaceutical compositions disclosed herein are administered to the central retina of a subject.

The dose of a vector of the invention may be determined according to various parameters, especially according to the age, weight and condition of the patient to be treated, the particular ocular disorder and the degree to which the disorder, if progressive, has developed, the route of administration; and the required regimen. Again, a physician will be able to determine the required route of administration and dosage for any particular patient. An effective amount of an rAAV carrying a nucleic acid sequence encoding the desired transgene under the control of the promoter sequence desirably ranges between about 1×10⁹ to 2×10¹² rAAV genome particles or between 1×10¹⁰ to 2×10¹¹ genome particles. A genome particle is defined herein as an AAV capsid that contains a single stranded DNA molecule that can be quantified with a sequence specific method (such as real-time PCR). In some embodiments, the about 1×10⁹ to 2×10¹² rAAV genome particles are provided in a volume of between about 150 to about 800 μl. In some embodiments, the about 1×10¹⁰ to 2×10¹¹ rAAV genome particles are provided in a volume of between about 250 to about 500 μl. Still other dosages in these ranges may be selected by the attending physician.

The dose may be provided as a single dose, but may be repeated for the fellow eye or in cases where vector may not have targeted the correct region of retina for whatever reason (such as surgical complication). The treatment is preferably a single permanent treatment for each eye, but repeat injections, for example in future years and/or with different AAV serotypes may be considered. As such, it may be desirable to administer multiple “booster” dosages of the pharmaceutical compositions disclosed herein. For example, depending upon the duration of the transgene within the ocular target cell, one may deliver booster dosages at 6 month intervals, or yearly following the first administration. Such booster dosages and the need therefor can be monitored by the attending physicians, using, for example, the retinal and visual function tests and the visual behavior tests known in the art. Other similar tests may be used to determine the status of the treated subject over time. Selection of the appropriate tests may be made by the attending physician. Still alternatively, the methods disclosed herein may also involve injection of a larger volume of a vector-containing solution in a single or multiple infection to allow levels of visual function close to those found in WT retinas.

### Additional Methods

In one aspect, provided is a method of increasing expression of Kv8.2 in a subject in need thereof, the method comprising administering to the subject a vector disclosed herein. In one aspect, provided is a method of increasing expression of Kv8.2 in a cell, the method comprising contacting the cell with a vector disclosed herein.

### Articles of Manufacture and Kits

Also provided are kits or articles of manufacture for use in the methods described herein. In aspects, the kits comprise the compositions described herein (e.g., compositions for delivery of a Kv8.2 coding sequence) in suitable packaging. Suitable packaging for compositions (such as ocular compositions for injection) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Also provided are kits comprising the compositions described herein. These kits may further comprise instruction(s) on methods of using the composition, such as uses described herein. The kits described herein may further include other materials desirable from a commercial and user standpoint, including buffers, diluents, filters, needles, syringes, and package inserts with instructions for performing the administration of the composition or performing any methods described herein. For example, in some embodiments, the kit comprises an rAAV comprising a KCNV2 transgene for the expression of a Kv8.2 protein in target cells, a pharmaceutically acceptable carrier suitable for injection, and one or more of: a buffer, a diluent, a filter, a needle, a syringe, and a package insert with instructions for performing the injections.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: AAV-KCNV2 Expression Construct Design and Production

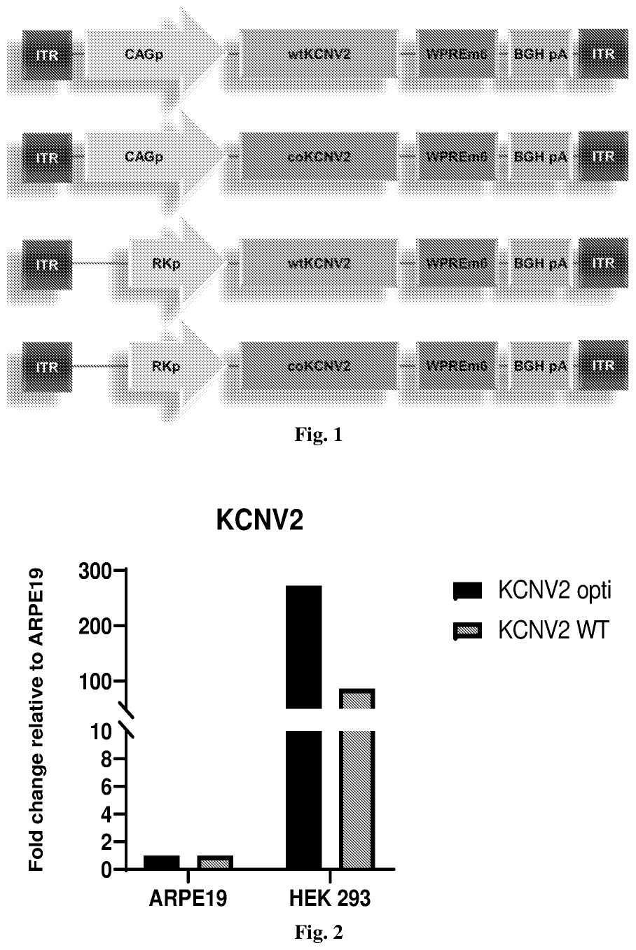

First, KCNV2 cDNA or the codon optimized KCNV2 cDNA were cloned into an AAV single stranded backbone downstream of the ubiquitous CAG promotor or photoreceptor-specific RK1 promoter. Kozak consensus sequences were placed between the promoter and the transgene. A Woodchuck Hepatitis Virus mutant 6 (WPREm6) sequence was placed between the transgene and the polyA. The polyA sequence was a Bovine growth hormone polyA (BghpA) sequence. See FIG. 1 for schematic representations of the four expression constructs. Cloning was carried out at VectorBuilder, Inc. (Chicago, IL, USA). Upon receipt of the plasmids, complete sequencing (including the ITR regions) was carried out at Genewiz (South Plainfield, NJ, USA) and sequences were aligned to the plasmid maps using Snapgene (San Diego, CA, USA). The four constructs were packages into both AAV5 and 7m8 capsids by triple transfection in HEK293 T cells and purified by cesium chloride centrifugation at SignaGen laboratories (Frederick, MD, USA).

### Example 2: Verification of CAG Expression Constructs by Transfection in Cell Lines

In order to validate the transgenic expression constructs, HEK293 and arising retinal pigment epithelial (ARPE19) cells were transfected using a standard nucleofection technique. Initially, cells were transfected with expression constructs comprising the WT KCNV2 gene or the codon optimized KCNV2 gene, respectively, under control of the CAG promotor (pCAG-KCNV2 WT and pCAG-KCNV2 Opti). An expression plasmid comprising a Green Fluorescent Protein (GFP) transgene and a human cytomegalovirus (CMV) promoter (CMV-GFP) was used as a control. Expression was verified by qPCR, immunofluorescence, and FACS.

Verification of CAG Expression Constructs by qPCR.

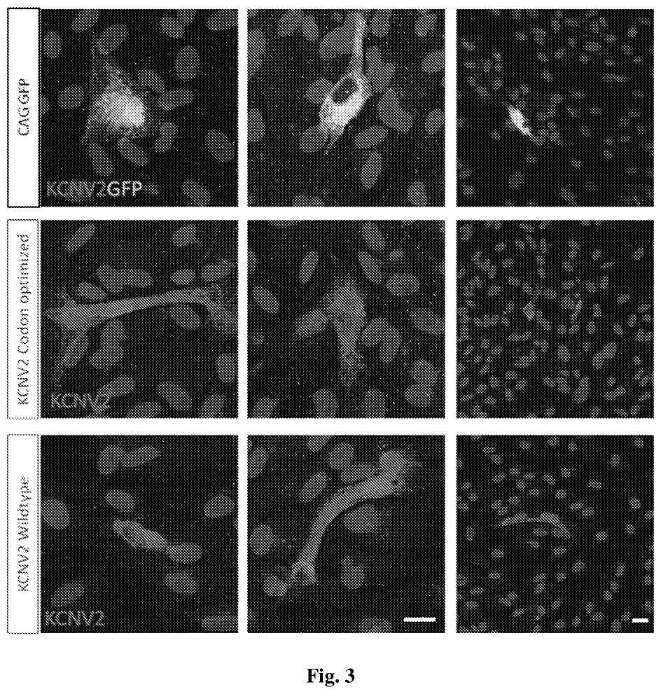

KCNV2 WT and KCNV2 Opti mRNA levels were assessed 48 hours post nucleofection of HEK293 or ARPE19 cells, respectively, with the pCAG-KCNV2 WT or pCAG-KCNV2 Opti expression constructs, respectively. mRNA levels were determined by qPCR using TAQMAN primer probe sets designed to detect WT and Opti transcripts. Expression levels were normalized to housekeeping genes GAPDH and β actin. Both expression plasmids produced detectable KCNV2 mRNA in both cell lines. Despite transfection of the same quantity of plasmid DNA, ARPE19 had less of both WT and Opti transcripts than HEK293 at 48 hours, suggesting poorer transfection efficiency (FIG. 2). Opti and WT transcript levels cannot be compared directly in this analysis due to differences in amplification efficiency between primer pairs.

### Verification of CAG Expression Constructs by Immunofluorescence.

ARPE19 cells were nucleofected with pCAG-KCNV2 WT and pCAG-KCNV2 Opti expression constructs, respectively. ARPE19 cells were also transfected with a pmaxGFP (GFP driven by a CAG promoter) expression construct from Lonza Biosciences (Morrisville, NC, USA) as a control. Kv8.2 protein was detected using a KCNV2 rabbit polyclonal primary antibody (Sigma Aldrich #HPA031131, 1:100) and a donkey anti-rabbit Alexa Fluor 555 secondary antibody. Certain ARPE19 cells transfected with both KCNV2 expression plasmids produced Kv8.2 protein that was detectable by immunofluorescence (FIG. 3). The transfection levels were low, as indicated by many ARPE19 cells that did not have detectable Kv8.2 protein. Kv8.2 protein localized to the cell membrane and cytoplasm.

### Verification of CAG Expression Constructs by Fluorescence-Activated Single Cell Sorting (FACS)

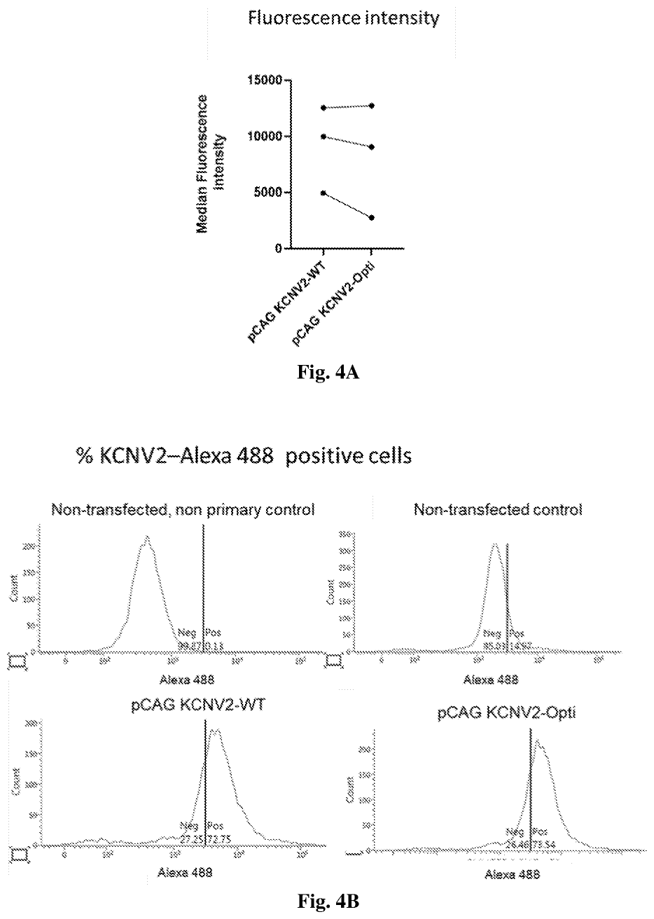

HEK293 cells were transfected with 3.5 μg pCAG-KCNV2 WT or pCAG-KCNV2 Opti expression constructs and harvested 48 hours later. A pCMV-GFP expression construct was used as a control. Cells were stained in suspension with Kv8.2 primary antibody (Sigma Aldrich #HPA031131, 1:100) and Alexa Fluor 488 anti-rabit antibody. Cell populations were gated against a non-transfected control (FIG. 4). There were no significant differences in the number of Kv8.2 expressing cells in WT vs. codon optimized plasmids at 48 hours (n=3 independent experiments).

Median fluorescence intensity (MFI) was used to quantify Kv8.2 protein expression levels in transfected cells. There was no significant differences between the median fluorescence of Kv8.2/Alexa Fluor 488 stained cells in KCNV2 WT vs KCNV2 Opti expression constructs at 48 hours (n=3 independent experiments).

### Example 3: AAV Transduction of Expression Constructs in ARPE19 Cells

AAV5 KCNV2 vectors (CAG-KCNV2 WT, CAG-KCNV2 Opti, RK-KCNV2 WT, and RK-KCNV2 Opti) were transduced into ARPE19 cells on chamber slides at two Multiplicities of Incection (MOIs) (1E4 vector genomes (VGs) per cell and 1E5 VGs per cell) and fixed 21 days later. Cells stained with Kv8.2 primary antibody and Alexa Fluor 555 secondary antibody were imaged by confocal. Three images (from three wells) per condition were taken and blinded before analysis in FIJI (Image J). The percent of Kv8.2 expressing cells were scored relative to DAPI and average staining intensity (integrated density) of Kv8.2 expressing cells was quantified in FIJI (Image J).

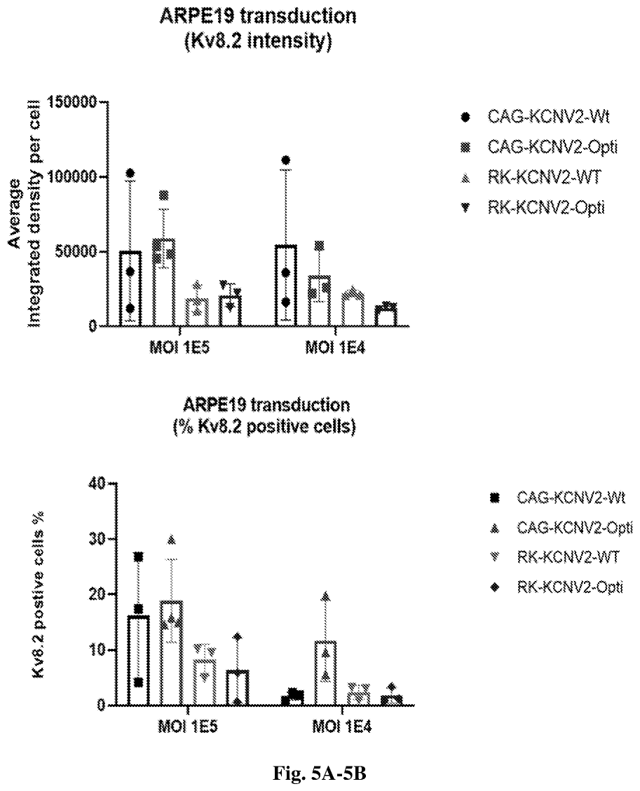

CAG promoter expressing AAVs (Opti and WT) scored higher in the percent Kv8.2 expressing cells and in the Kv8.2 staining intensity levels than RK (Opti and WT). CAG Opti and CAG WT vectors did not have significantly different staining intensity levels in Kv8.2 expressing cells in either MOI although the variability was high. CAG Opti had significantly more Kv8.2 positive cells in the 1E4 condition but not in the 1E5 (FIG. 5).

### Example 4: AAV Transduction of Expression Constructs in Organoids

Methods for AAV Transduction of Organoids with Expression Constructs

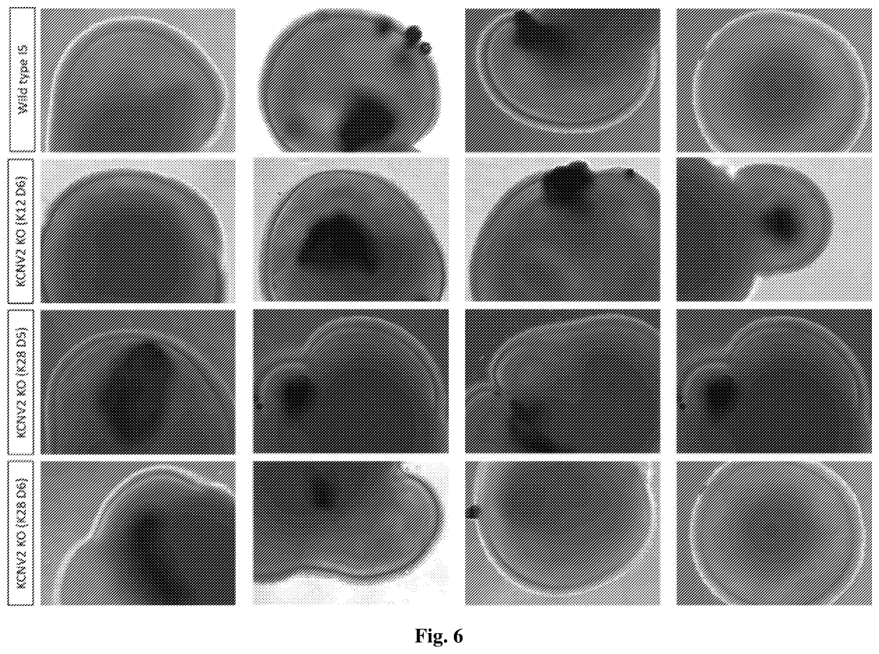

Retinal organoids were transferred into 96 well low-attachment plates (one organoid per well) and transduced at day 140 with one of the eight AAV KCNV2 constructs (AAV5 CAG-KCNV2 WT, AAV5 CAG-KCNV2 Opti, AAV5 RK-KCNV2 WT, AAV5 RK-KCNV2 Opti, AAV7m8 CAG-KCNV2 WT, AAV7m8 CAG-KCNV2 Opti, AAV7m8 RK-KCNV2 WT, and AAV7m8 RK-KCNV2 Opti) at a dose of 3E11 viral genomes (VG) per organoid in a total of 100 μL of media. The following day, organoids were transferred into 24 well low-attachment plates and the media was changed 3 days later. Retinal organoids for transduction were selected based on morphology; the presence of a clear laminated structure and visible outer segments brush borders (FIG. 6) were selected for fixation and analysis by immunofluorescence. Organoids with an internal rosette structure (in which photoreceptors are present in internal structures) were transduced for qPCR analysis in which mRNA from the whole organoid is assayed.

Organoids were cultured for a further 3 weeks before harvesting by snap freezing of whole organoids (qPCR and western blot) or fixing in 4% paraformaldehyde (PFA) for 30 minutes at 4° C. Next, organoids were washed twice in standard phosphate buffered saline solution (PBS) and subsequently immersed in PBS with 30% sucrose overnight at 4° C. The following day, organoids were embedded in optimal cutting temperature (OCT) compound and stored at −80° C. before cryosectioning at 7 μm.

For each transduction of KCNV2 KO organoids, a non-transduced control from the same clone and differentiation batch and a WT (non-CRISPR edited) control was included.

AAV7m8 KCNV2 transduction in outermost layer of photoreceptor cells

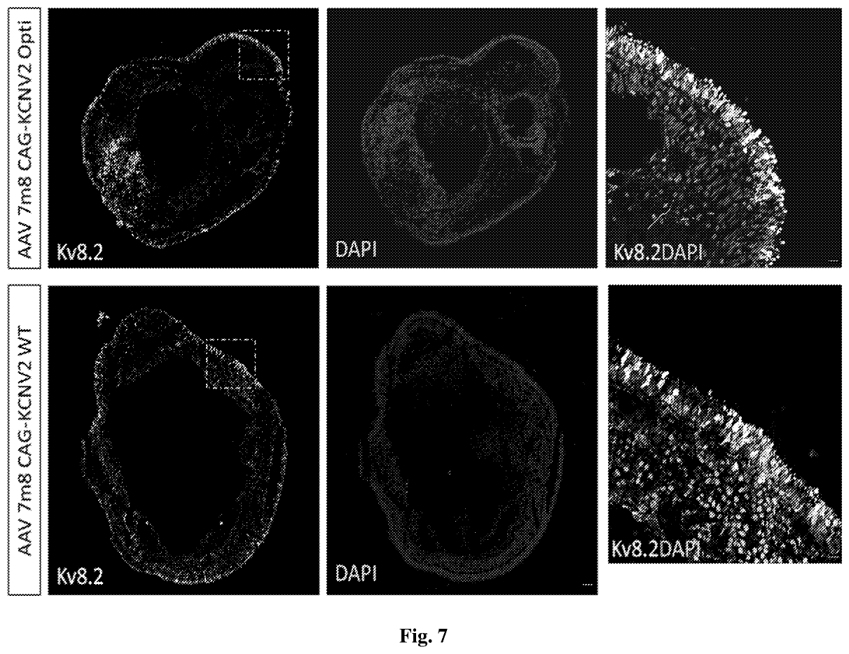

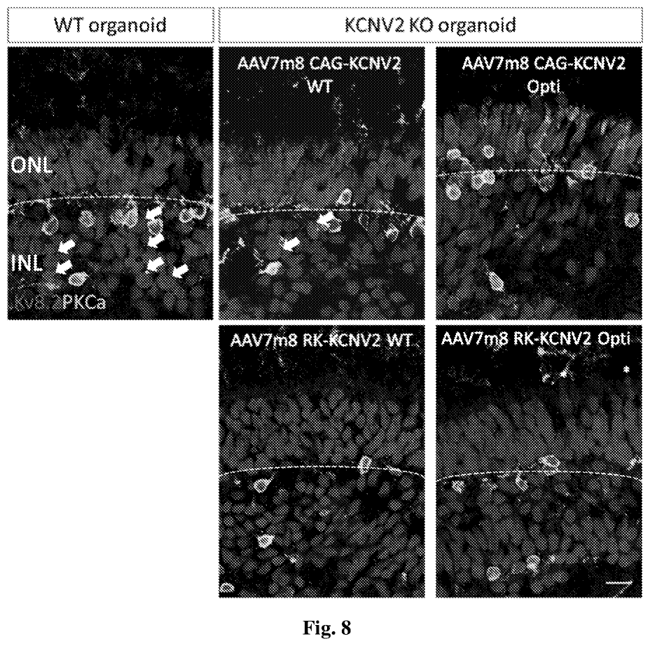

Three weeks post AAV transduction KCNV2 KO retinal organoids were sectioned and assayed for transgenic KCNV2 protein product Kv8.2. Confocal analysis of whole retinal organoids revealed that both KCNV2 WT and KCNV2 codon optimized vectors were expressed in the outermost, photoreceptor layer (FIG. 7).

### AAV7m8 KCNV2 Transduction of Inner Retinal Cells

There was little detectable Kv8.2 product in inner retinal layers in transduced organoids. Co-staining with bipolar cell markers PKCa revealed no Kv8.2 staining PKCa positive bipolar cells, however WT retinal organoids had several inner retinal cells immune-positive for Kv8.2 (white arrows FIG. 7), possibly amacrine, horizontal or cone bipolar cells. In contrast, transduced KCNV2 KO retinal organoids had very few Kv8.2 positive inner retinal cells despite the high expression in transduced photoreceptors in CAG promoter containing vectors (FIG. 8), indicating an inability of the AAV to access these layers and/or preferential vector tropisms for photoreceptor cells.

### AAV7m8 KCNV2 Transduction of Retinal Pigment Epithelial (RPE) Cells

Pigmented RPE cells and photoreceptors originate from the same developmental progenitor cell population. In vivo, the RPE monolayer lies adjacent to the photoreceptor outer segments defining the boundary of the sub-retinal space. RPE cells in retinal organoids are arranged in clusters on the external surface of the organoid (FIG. 9, left panel). Where present, RPE cells were transduced by both the AAV5 and 7m8 and CAG-KCVN2 expressed high levels of Kv8.2 protein. RK-KCNV2 did not express detectable Kv8.2 in RPE cells, possibly due to the photoreceptor specificity of the RK promoter.

### AAV7m8 KCNV2 Transduction of Muller Glia Cells

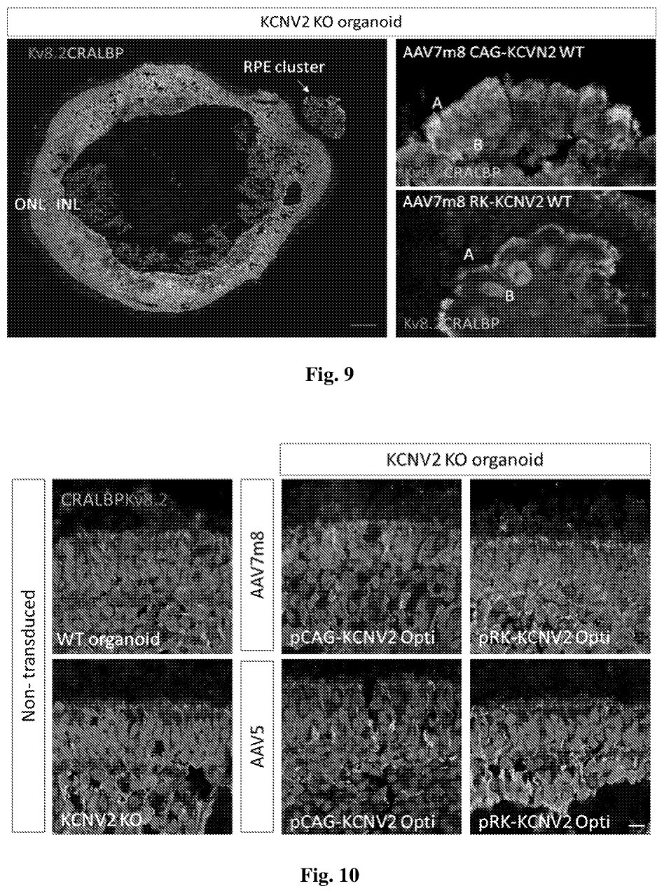

Muller Glia cells span the full thickness of the retina providing architectural support and forming the outer and inner limiting membrane. In addition to RPE cells, CRALBP is a marker for Muller Glia in retinal organoids which can be seen spanning the inner and outer nuclear layers and forming the outer limiting membrane. Co-staining with CRALBP with Kv8.2 revealed no co-staining of these two markers, suggesting that the AAV5 and AAV7m8 does transduce and/or express the transgene in Muller glia cells (FIG. 10).

### Example 5: Kv8.2 Localization in AAV7m8 KCNV2 Transduced Cells

### Kv8.2 Localization in Photoreceptor Inner Segments

Endogenous KCNV2 (Kv8.2) protein is reportedly expressed in the plasma membrane of rod and cone inner segments and not in outer segments. The trafficking of photoreceptor proteins to their correct subcellular compartment is critical to their function, and the mis-trafficking of incorrectly folded proteins underlies the pathogenicity of many inherited retinal degenerative disorders.

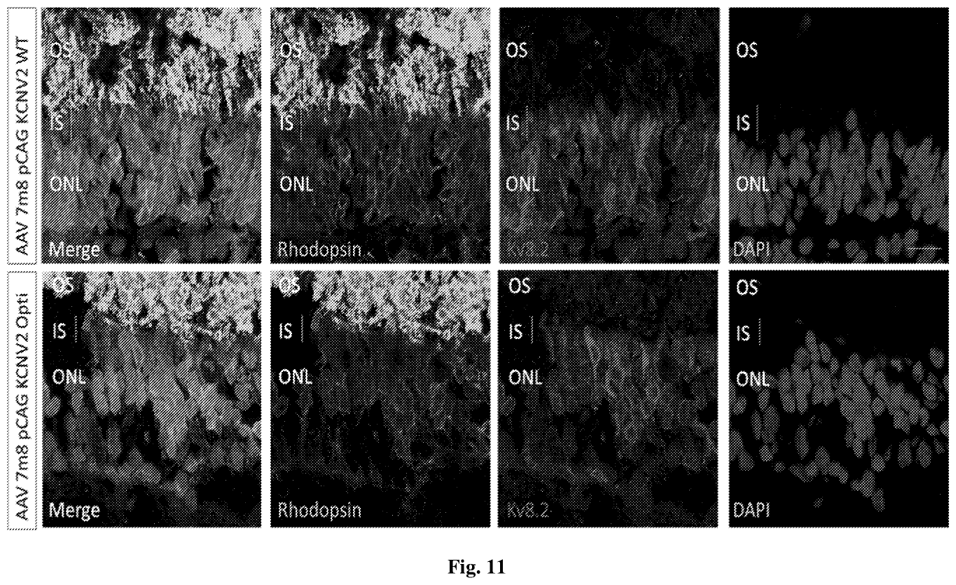

Transduced retinal organoids were stained with rhodopsin, which was found to correctly localize to the membranous outer-segment structures. Transgenic kv8.2 (7m8 CAG-WT and 7m8 CAG-Opti) was found to localize to the inner segments (IS) and plasma membrane of photoreceptor cell bodies (FIG. 11). Kv8.2 staining was absent from outer segments (OS). This suggests that the protein generated from both WT and codon optimized vectors are appropriately trafficked post translation.

Kv8.2 Colocalization with Kv2.1 in AAV7m8 KCNV2 Transduced Cells

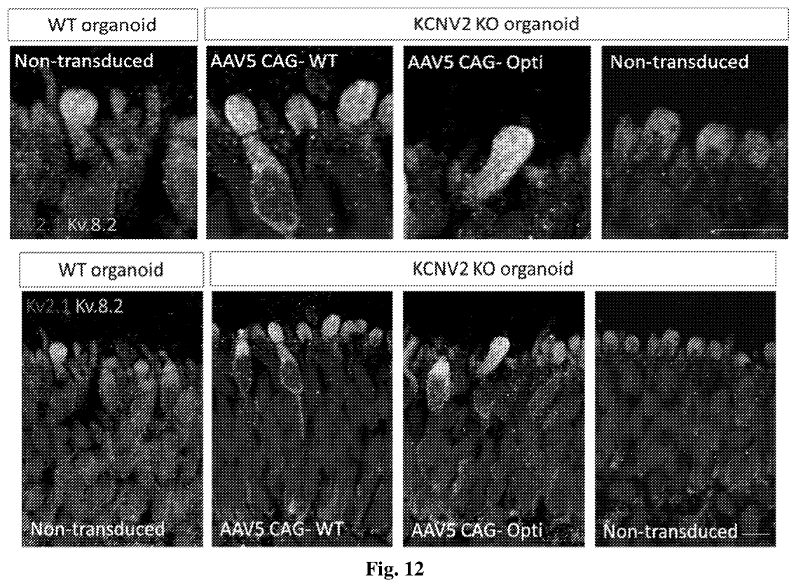

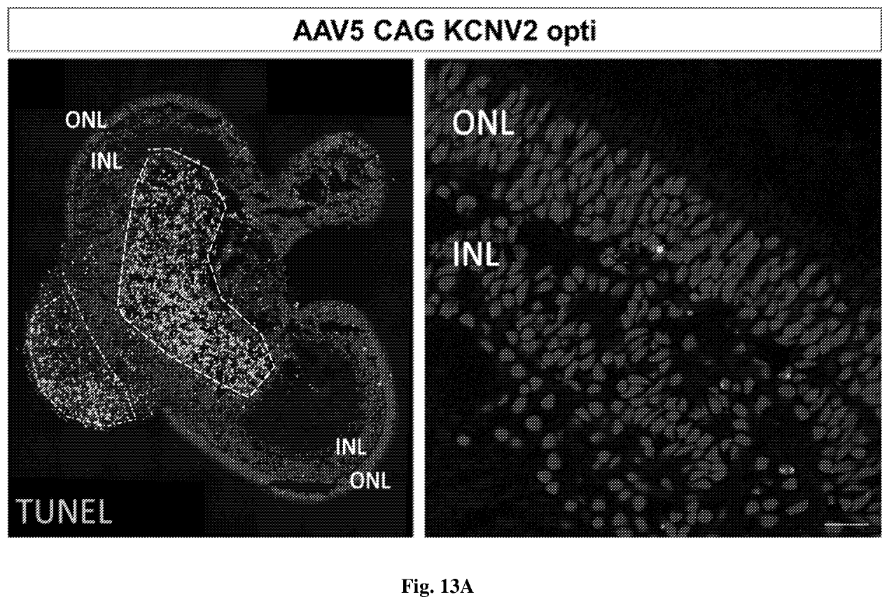

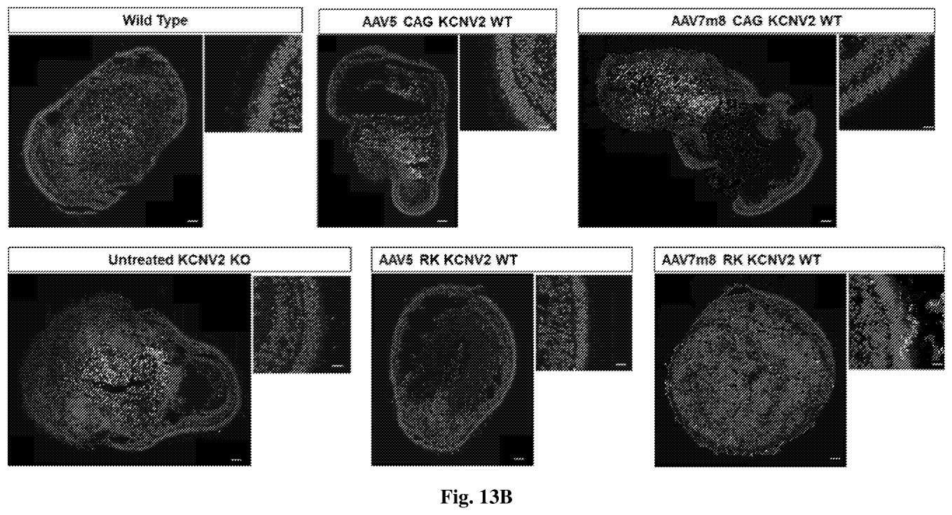

KCVN2 gene product Kv8.2 interacts with potassium channel subunit Kv2.1 in the retina. Kv8.2 is a silent Kv channel subunit and thus can only function via its interaction with larger Kv channel subunits. In WT retinal organoids, aKv2.1 antibody clearly labelled photoreceptor inner segments with a stronger signal in the cone inner segments (ellipsoid region) (FIG. 12). Endogenous Kv8.2 protein (FIG. 12) was present in rod and cone inner segments where is co-localised with Kv2.1. In KO retinal organoids, where Kv8.2 is absent, the inner segment ellipsoid pattern of Kv2.1 staining is detected photoreceptors. AAV derived Kv8.2 protein (both WT and codon optimized vectors) was also expressed in photoreceptor inner segment structures in transduced retinal organoids indicating that both transgenes are translated into protein which is efficiently trafficked to the correct subcellular compartment (FIG. 12).

### Example 6: Assessment of Rescue and Toxicity after AAV Transduction

An increase in TUNEL reactivity across the retina has been reported in KCNV2 KO mouse model at 1, 3 and 6 months of age and a reduction in cone cell number per mm2 to 80% of WT was reported at 6 months. To determine whether our fetal stage KCNV2 KO retinal cell model recapitulated these phenotypes at the time of transduction, and to assess any vector associated cytotoxicity, TUNEL reactivity and cone cell numbers were measured in WT vs. KO organoids and KO organoids transduced with all AAV vectors.

### TUNEL Reactivity in AAV Transduced Organoids

TUNEL is a method for detecting DNA fragmentation by labelling the 3′-hydroxyl termini in the double-strand DNA breaks generated during apoptosis. TUNEL reactivity in retinal organoid cryosections was assessed in KCNV2 KO transduced organoids vs. non transduced controls and WT.

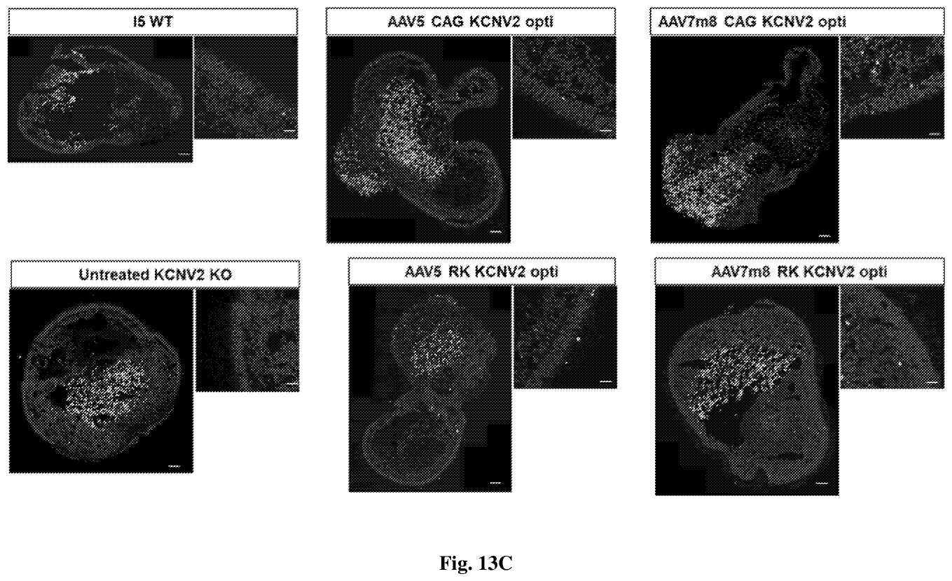

FIG. 13A shows TUNEL staining of AAV5 CAG-KCNV2-Opti treated retinal organoids (clone K28), 3 weeks post transduction. TUNEL positive cells were mostly noted in the centre of the organoid (dashed line) with no or very few TUNEL positive cells in the retinal cell layers (ONL, INL). There were no increases in TUNEL reactivity in KCNV2 KO photoreceptors relative to WT, suggesting that ‘in vitro’ retinal degeneration was not occurring at this time point in this model.

Neither of the two AAV serotypes caused a significant level of TUNEL positive cells in the ONL or INL with WT or codon optimised transgenes (FIGS. 13b and 13c). This suggests that the tested AAV serotypes and overexpressed transgenic protein are not cytotoxic to retinal cells. The presence of TUNEL positive cells in the centre of the organoid has been widely reported in other HIPSC retinal organoid models and is most likely due to hypoxia and/or poor nutrient transfer to cells in the centre of the retinal organoid.

### Clone Cell Number in AAV Transduced Organoids

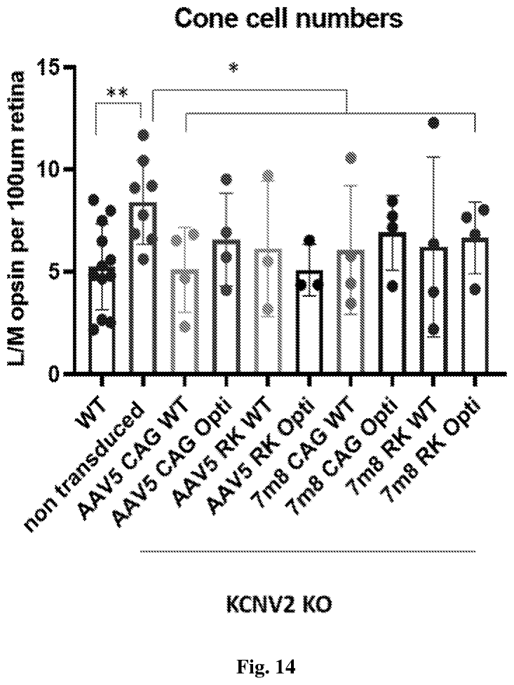

KCNV2 KO mice exhibit a mild loss of cone cells at 6 months of age to 80% of WT levels. To determine if this phenotype is recapitulated in human fetal stage retinal organoids, cone cells per 100 μm of retinal tissue were quantified by immunofluorescence in WT and KCNV2 KO retinal organoids. L/M opsin positive cone cell numbers were counted from whole organoid tile scans taken at 40× magnification (7 μm retinal cryosection) and normalized to the total length of retinal tissue. Average cone cell numbers were counted in total of 12 WT and 8 untreated KO retinal organoids. There was a significant increase in cone cell numbers in KCNV2 KO cell lines relative to WT (FIG. 14). Transduced retinal organoids (all vectors grouped n=30) showed no statistically significant difference between WT and transduced organoids p=0.2 but a significant reduction relative to non-transduced KO (p=0.02).

### Example 7: Quantitative Assessment of Transgenic KCNV2 mRNA and Kv8.2 Protein in Transduced Organoids

qPCR Assessed KCNV2 mRNA Levels in Transduced Organoids

Quantitative comparison of vector driven transgene expression was carried out by qPCR. KCNV2 mRNA expression levels were assessed in KCNV2 KO organoids transduced with WT and codon optimised version of the KCNV2 gene driven by a RK or CAG promoter and delivered either by AAV2/5 or AAV2/7m8. Whole transduced organoids from clones K12, K5 and K28 were harvested 21 days post transduction by snap freezing. RNA was extracted, DNAse treated and cDNA made from 0.1 μg of RNA according to SOP (PRCL-SOP-RNA purification cDNA synthesis). Gene expression levels were normalised to endogenous housekeeping genes GAPDH and β actin and relative expression was determined using the ΔΔCT method.

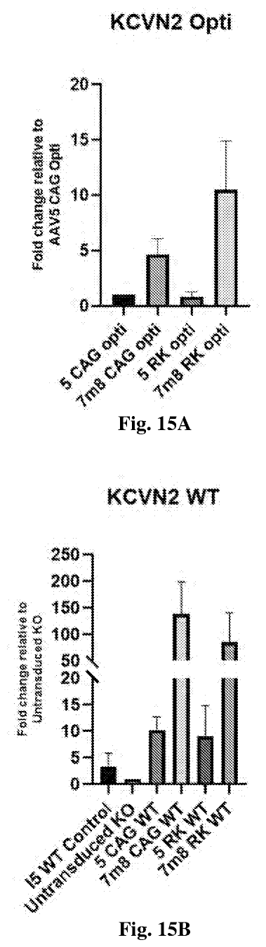

Highest level of KCNV2-Opti expression were observed in retinal organoids transduced with AAV7m8-RK-codon optimised KCNV2 (FIG. 15a) compared to retinal organoids that received either AAV5-RK-codon optimised KCNV2, AAV5-CAG-codon optimised KCNV2, or AAV7m8-CAG-codon optimised.

Highest levels of vector derived KCNV2 WT mRNA were observed in organoids transduced with AAV7m8-CAG-WT KCNV2. KCNV2 expression in organoids treated with AAV7m8-CAG-WT KCNV2 was ˜138 fold higher than non-transduced KCNV2 KO control (FIG. 9b) and AAV7m8-RK-WT KCNV2 was ˜86 fold higher than non-transduced control. Retinal organoids transduced with the different versions of the KCNV2 WT gene delivered with the AAV5, either under the control of a CAG promoter or RK promoter were ˜10 fold higher than non-transduced controls (FIG. 15B).

Overall, AAV2-7m8 was found to be more efficacious in transducing photoreceptors in retinal organoids than AAV5. Interestingly there was no significant difference in either WT or Opti KCNV2 mRNA in vectors driven by the photoreceptor specific RK promoter or the constitutive CAG promoter.

### Kv8.2 Protein Levels in Transduced Organoids, Assessed by Immunofluorescence.

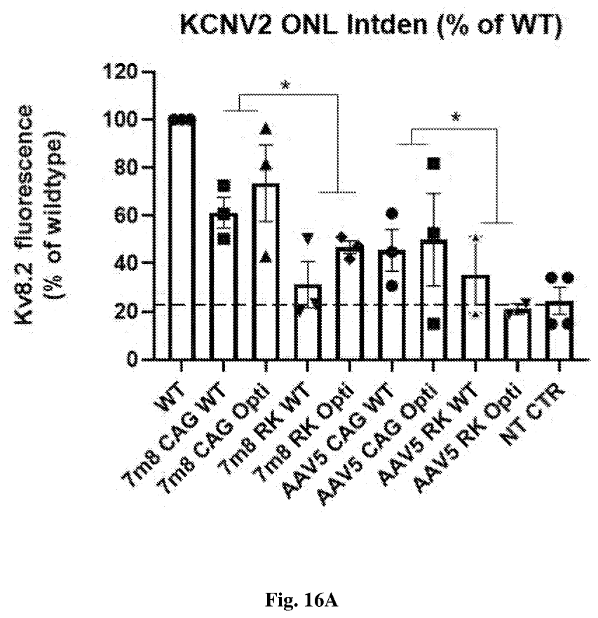

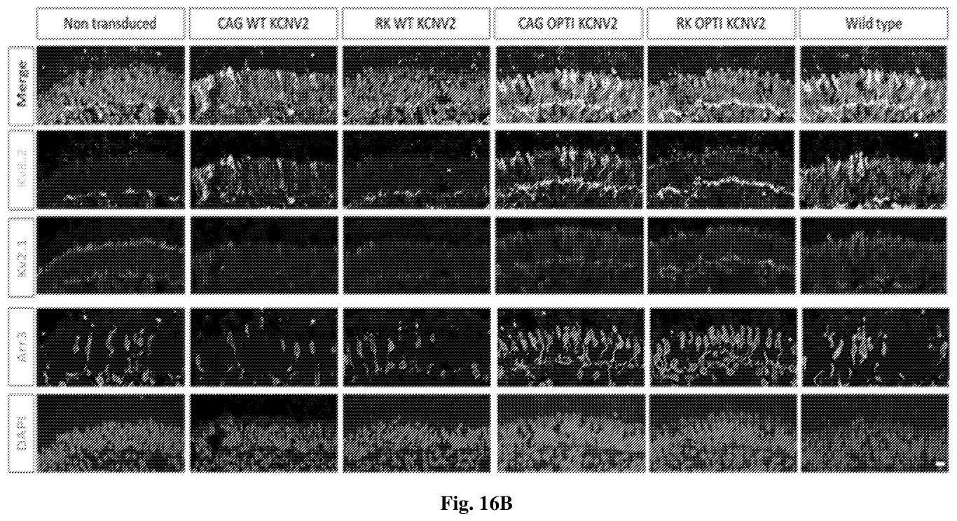

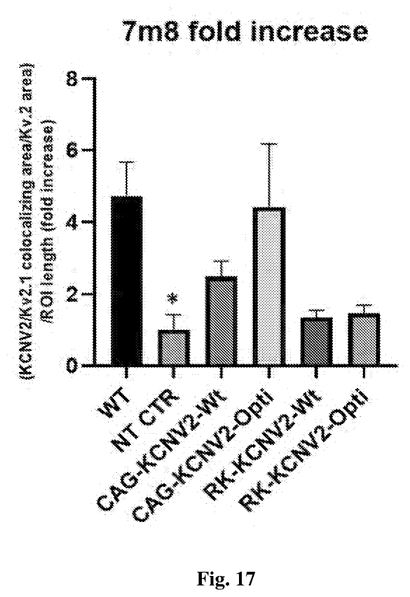

Kv8.2 protein levels were expressed in the outer nuclear layer of transduced organoids (see FIG. 7, FIG. 16B). In order to determine relative cumulative protein levels between vectors, organoid cryosections were stained with Kv8.2 antibody and the total cumulative fluorescence in the ONL (raw integrated density) was quantified in FIJI (Image J) and normalised to the total ONL area measured. FIG. 16 shows the total fluorescence expressed as a percentage of WT organoids embedded on the same block and imaged on the same day. There was a significant difference in total fluorescence between CAG and RK promoters in both 7m8 and AAV5 capsids (p=0.031 and 0.028 respectively, 2 tailed, paired student's t test). There was no significant difference between WT and Opti vectors with CAG or RK promoters despite a trend towards increased fluorescence intensity in codon optimized (Opti) vectors.

### Example 8: Colocalization of Kv8.2 and Kv2.1 in Transduced Retinal Organoids

### Relative Colocalization of Kv8.2 and Kv2.1 Assessed by Immunofluorescence

Kv8.2 functions in the retina as by forming a heteromer with voltage gated potassium channel Kv2.1. Qualitative analysis revealed co-localisation of vector derived Kv8.2 with endogenous Kv2.1 at the photoreceptor inner segment (FIG. 12). In order to determine relative levels of restored Kv8.2, quantitative immunofluorescence and co-localisation analysis were carried out. 7 μm organoid cryosections were co-stained with Kv8.2 and Kv2.1 and whole organoid sections were imaged at 40× magnification and subsequently merged in LSM software to create a tile scan of the whole organoid which was exported into FIJI image analysis software. Tile scans of n=3-5 organoids per vector were acquired and analysed in FIJI (image J). The total co-localising area in the inner segment region was determined using the “image calculate> and” function to determine pixels above threshold in both Kv.2.1 and Kv8.2 channels. This value was normalised to the length of the region of interest to account for the varying retinal organoid sizes.

There was a significant difference between WT (CTR) and non-treated KCNV2 KO organoids (FIG. 16). There was a trend towards an increase in average co-localising area in 7m8 CAG-WT and 7m8 CAG-Opti treated organoids, but the difference did not reach significance (one way ANOVA, Dunnett's multiple comparison test). 7m8 RK-WT and RK-Opti average co-localising area was similar to non-treated indicating a lack of vector derived Kv8.2 expression detectable by this method.

### Colocalization and Proximity of Kv8.2 and Kv2.1 Assessed by a Proximity Ligation Assay

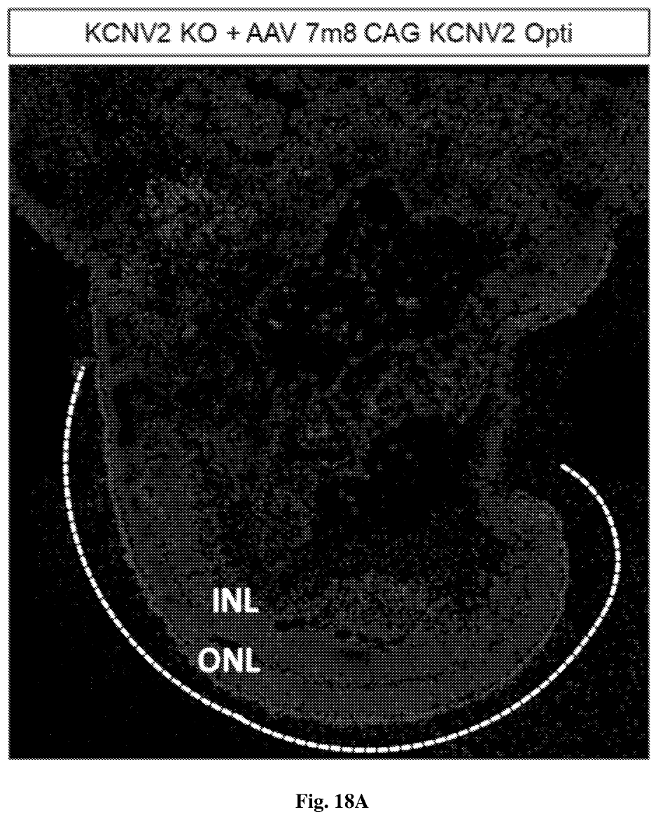

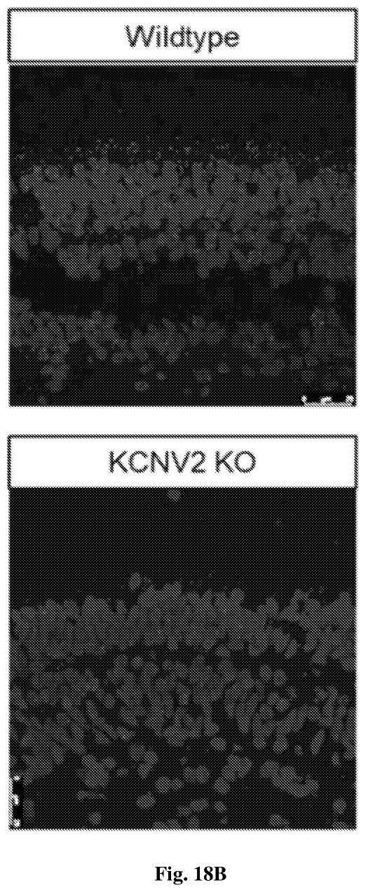

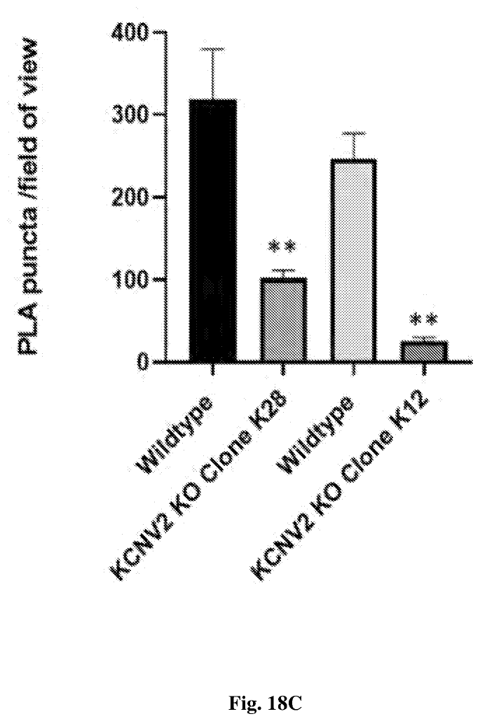

A proximity ligation assay (PLA) was developed to assess protein-protein interactions in photoreceptors between potassium channel subunits Kv8.2 and Kv2.1. Transduced KCNV2 KO retinal organoids along with WT (positive control) and non-transduced KO (negative control) were fixed and embedded in OCT on the same block for cryosectioning. 7 μm cryosections were co-stained with Kv8.2 (rabbit) and Kv2.1 (mouse) antibodies and rabbit and mouse PLA plus and minus probes. Following ligation and amplification steps (duo link-orange), PLA puncta in the outer nuclear layer were visualised at 63× magnification by confocal microscopy. Observing the organoid as a whole there was a clear concentration of PLA signal at the location of the photoreceptor cell layer, specifically where the photoreceptor inner segments were located (FIG. 18A). This confirms the specificity of the kv.8.2/Kv2.1 interaction in the expected cell type and subcellular compartment. Further, specificity was confirmed by the significant reduction in signal at the ONL of KCNV2 KO retinal organoids (FIG. 18B, 18C). Quantification of PLA signal revealed a significant reduction in PLA puncta number per area of ONL in KCNV2 KO clones (K28 and K12) relative to WT organoid processed on the same slide (FIG. 18C).

### Quantification of PLA Signal in Transduced Retinal Organoids

KCNV2 KO retinal organoids transduced with AAV vectors express KCNV2 mRNA and Kv8.2 protein. The function of vector derived translated protein is dependent of its ability to form a heteromer with voltage gated potassium channel kv2.1. In addition to assessing the total quantity of vector derived KCNV2 transcript and Kv8.2 in protein we used PLA to assess the extent of its interactions with Kv2.1.

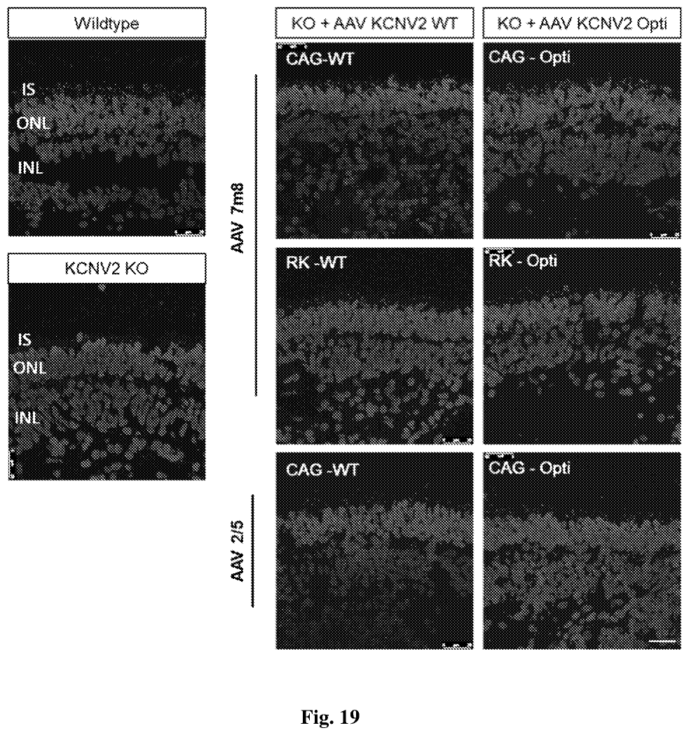

KCNV2 KO organoids transduced with one of the 8 indicated vectors from clonal lines were embedded in the same cryopreserved tissue block as WT (positive control) and non-transduced KCNV2 KO organoids (negative control), the experiment was repeated in KCNV2 KO clonal cell lines K12 and K28. As above 7 μm cryosections were co-stained with Kv8.2 (rabbit) and Kv2.1 (mouse) antibodies and rabbit and mouse PLA plus and minus probes. Maximum intensity z projections at 63× magnification were used to quantify PLA puncta per field of view. Each z projection captured 100-150 photoreceptor cells and contained between 17 (non-transduced) and 550 puncta (maximum signal). The Kv8.2 antibody titre was reduced from 1 in 100 to 1 in 400 to maximise signal without coalescence of PLA puncta. FIG. 19 shows representative maximum intensity projections from KCNV2 KO clone K12 transduced with both AAV 2/5 and AAV7m8 capsids vs WT and non-transduced. PLA puncta were more abundant in transduced organoids relative to non-transduced, with the signal frequency highest at the apical edge in the region of photoreceptor inner segments. Puncta in the ONL were quantified using FIJI (Image J) ‘analyse particles’ function. Regional differences in ONL size necessitated the normalisation of signal to the ONL area measured.

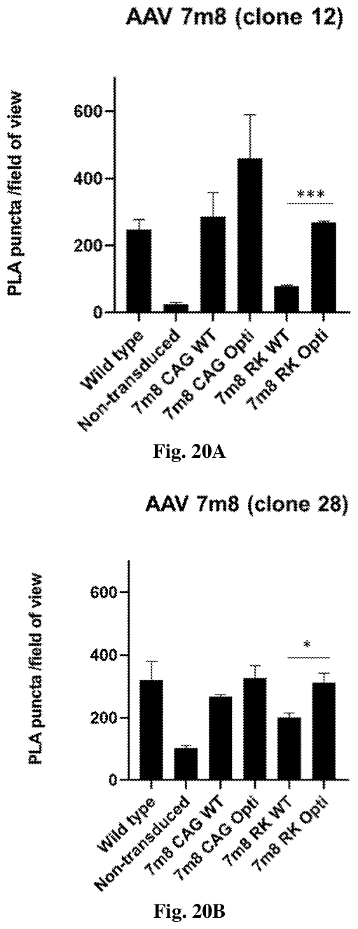

There was a significant effect of AAV transduction on PLA puncta number (P>0.001, one way ANOVA) individual comparisons showed that all vectors produced a significantly higher signal than non-transduced (FIGS. 20A and 20B). CAG WT vectors produced a significantly higher PLA signal than RK WT vectors (K12 p=0.02, K28 P=0.01) but there was no significant different between CAG Opti and RK Opti vectors (K12 p=0.17, K28 p=0.77). In both clones there was a significant difference between 7m8 RK Wt and 7m8 RK Opti (K12 p<0.0001, K28 p>0.01).

Higher PLA signal in KCNV2 KO photoreceptors is an indicator of higher functional protein levels. In all 7m8 vectors the signal did not differ significantly from the WT levels—with the exception of 7m8 RK-WT in clone K12.

This is an indication that all 7m8 vectors are able to deliver KCNV2 to human photoreceptor cells with sufficient efficacy that enables the restoration of functional Kv2.1/Kv8.2 heteromers to WT levels.

### Overview of Sequences

| SEQ | | | |
|---|---|---|---|
| ID | | | |
| NO | Name | Description | Sequence |
| 1 | AAVss- | AAV2 5′ | CCTGCAGGCAGCTGCGCGCTCGCTCGCTCACTGA |
| | CAG- | ITR: | GGCCGCCCGGGCAAAGCCCGGGCGTCGGGCGAC |
| | KCNV2- | 1-141 bp | CTTTGGTCGCCCGGCCTCAGTGAGCGAGCGAGCG |
| | OPTI | CAG: | CGCAGAGAGGGAGTGGCCAACTCCATCACTAGG |
| | | 169-1901 bp | GGTTCCTTCTAGACAACTTTGTATAGAAAAGTTG |
| | | Kozak: | CTCGACATTGATTATTGACTAGTTATTAATAGTAA |
| | | 1926-1931 bp | TCAATTACGGGGTCATTAGTTCATAGCCCATATA |
| | | KCNV2 | TGGAGTTCCGCGTTACATAACTTACGGTAAATGG |
| | | (opti) | CCCGCCTGGCTGACCGCCCAACGACCCCCGCCCA |
| | | 1932-3569 bp | TTGACGTCAATAATGACGTATGTTCCCATAGTAA |
| | | WPREmut6: | CGCCAATAGGGACTTTCCATTGACGTCAATGGGT |
| | | 3570-4180 bp | GGAGTATTTACGGTAAACTGCCCACTTGGCAGTA |
| | | BGH pA: | CATCAAGTGTATCATATGCCAAGTACGCCCCCTA |
| | | 4235-4442 bp | TTGACGTCAATGACGGTAAATGGCCCGCCTGGCA |
| | | AAV2 3′ | TTATGCCCAGTACATGACCTTATGGGACTTTCCTA |
| | | ITR: | CTTGGCAGTACATCTACGTATTAGTCATCGCTATT |
| | | 4450-4590 bp | ACCATGGTCGAGGTGAGCCCCACGTTCTGCTTCA |
| | | | CTCTCCCCATCTCCCCCCCCTCCCCACCCCCAATT |
| | | | TTGTATTTATTTATTTTTTAATTATTTTGTGCAGCG |
| | | | ATGGGGGCGGGGGGGGGGGGGGGGCGCGCGCCA |
| | | | GGCGGGGCGGGGCGGGGCGAGGGGCGGGGCGGG |
| | | | GCGAGGCGGAGAGGTGCGGCGGCAGCCAATCAG |
| | | | AGCGGCGCGCTCCGAAAGTTTCCTTTTATGGCGA |
| | | | GGCGGCGGCGGCGGCGGCCCTATAAAAAGCGAA |
| | | | GCGCGCGGCGGGCGGGAGTCGCTGCGCGCTGCCT |
| | | | TCGCCCCGTGCCCCGCTCCGCCGCCGCCTCGCGC |
| | | | CGCCCGCCCCGGCTCTGACTGACCGCGTTACTCC |
| | | | CACAGGTGAGCGGGCGGGACGGCCCTTCTCCTCC |
| | | | GGGCTGTAATTAGCGCTTGGTTTAATGACGGCTT |
| | | | GTTTCTTTTCTGTGGCTGCGTGAAAGCCTTGAGGG |
| | | | GCTCCGGGAGGGCCCTTTGTGCGGGGGGAGCGGC |
| | | | TCGGGGGGTGCGTGCGTGTGTGTGTGCGTGGGGA |
| | | | GCGCCGCGTGCGGCTCCGCGCTGCCCGGCGGCTG |
| | | | TGAGCGCTGCGGGCGCGGCGCGGGGCTTTGTGCG |
| | | | CTCCGCAGTGTGCGCGAGGGGAGCGCGGCCGGG |
| | | | GGCGGTGCCCCGCGGTGCGGGGGGGGCTGCGAG |
| | | | GGGAACAAAGGCTGCGTGCGGGGTGTGTGCGTG |
| | | | GGGGGGTGAGCAGGGGGTGTGGGCGCGTCGGTC |
| | | | GGGCTGCAACCCCCCCTGCACCCCCCTCCCCGAG |
| | | | TTGCTGAGCACGGCCCGGCTTCGGGTGCGGGGCT |
| | | | CCGTACGGGGCGTGGCGCGGGGCTCGCCGTGCCG |
| | | | GGCGGGGGGTGGCGGCAGGTGGGGGTGCCGGGC |
| | | | GGGGCGGGGCCGCCTCGGGCCGGGGAGGGCTCG |
| | | | GGGGAGGGGCGCGGCGGCCCCCGGAGCGCCGGC |
| | | | GGCTGTCGAGGCGCGGCGAGCCGCAGCCATTGCC |
| | | | TTTTATGGTAATCGTGCGAGAGGGCGCAGGGACT |
| | | | TCCTTTGTCCCAAATCTGTGCGGAGCCGAAATCT |
| | | | GGGAGGCGCCGCCGCACCCCCTCTAGCGGGCGCG |
| | | | GGGCGAAGCGGTGCGGCGCCGGCAGGAAGGAAA |
| | | | TGGGCGGGGAGGGCCTTCGTGCGTCGCCGCGCCG |
| | | | CCGTCCCCTTCTCCCTCTCCAGCCTCGGGGCTGTC |
| | | | CGCGGGGGGACGGCTGCCTTCGGGGGGGACGGG |
| | | | GCAGGGCGGGGTTCGGCTTCTGGCGTGTGACCGG |
| | | | CGGCTCTAGAGCCTCTGCTAACCATGTTCATGCCT |
| | | | TCTTCTTTTTCCTACAGCTCCTGGGCAACGTGCTG |
| | | | GTTATTGTGCTGTCTCATCATTTTGGCAAAGAATT |
| | | | GCAAGTTTGTACAAAAAAGCAGGCTGCCACCATG |
| | | | CTGAAGCAGAGCGAGAGAAGGCGGAGCTGGTCC |
| | | | TACAGACCTTGGAACACCACAGAGAACGAGGGC |
| | | | AGCCAGCACAGAAGATCCATCTGTTCTCTGGGCG |
| | | | CCAGAAGCGGCTCTCAGGCCTCTATTCATGGCTG |
| | | | GACCGAGGGCAACTACAACTACTACATCGAAGA |
| | | | GGACGAGGACGGCGAGGAAGAGGACCAGTGGAA |
| | | | AGATGACCTGGCCGAGGAAGATCAGCAGGCCGG |
| | | | CGAAGTGACAACAGCCAAGCCTGAAGGACCTAG |
| | | | CGATCCTCCTGCTCTGCTGAGCACCCTGAATGTG |
| | | | AATGTCGGCGGCCACAGCTACCAGCTGGATTACT |
| | | | GTGAACTGGCCGGCTTTCCCAAGACCAGACTGGG |
| | | | CAGACTGGCCACCAGCACAAGCAGATCTAGACA |
| | | | GCTGAGCCTGTGCGACGACTACGAGGAACAGAC |
| | | | CGACGAGTACTTCTTCGACAGAGATCCCGCCGTG |
| | | | TTTCAGCTGGTGTACAACTTCTACCTGAGCGGCG |
| | | | TGCTGCTGGTGCTGGATGGACTGTGCCCTCGGAG |
| | | | ATTTCTGGAAGAACTCGGCTACTGGGGCGTCAGA |
| | | | CTGAAGTACACCCCTCGGTGCTGCCGGATCTGCT |
| | | | TCGAGGAAAGAAGGGACGAGCTGAGCGAGCGGC |
| | | | TGAAGATCCAGCATGAACTGAGAGCCCAGGCTCA |
| | | | GGTGGAAGAGGCCGAAGAACTGTTCCGGGACAT |
| | | | GAGATTCTACGGCCCTCAACGGCGGAGACTGTGG |
| | | | AACCTGATGGAAAAGCCTTTTAGCAGCGTGGCCG |
| | | | CCAAGGCCATTGGAGTGGCCTCTTCTACATTCGT |
| | | | GCTGGTGTCTGTGGTGGCCCTGGCTCTGAATACC |
| | | | GTGGAAGAGATGCAGCAGCACTCTGGCCAAGGC |
| | | | GAAGGCGGACCTGATCTGAGGCCTATCCTGGAAC |
| | | | ACGTGGAAATGCTGTGCATGGGCTTTTTCACCCT |
| | | | GGAATACCTGCTGCGGCTGGCCTCTACACCCGAC |
| | | | CTGAGAAGATTTGCCAGATCTGCCCTGAACCTGG |
| | | | TGGATCTGGTGGCTATCCTGCCTCTGTATCTGCAG |
| | | | CTGCTGCTGGAATGTTTTACCGGCGAGGGACATC |
| | | | AGAGGGGCCAGACAGTGGGATCTGTGGGCAAAG |
| | | | TTGGACAGGTGCTGAGAGTGATGCGGCTGATGAG |
| | | | AATCTTCCGGATCCTGAAGCTGGCCAGACACAGC |
| | | | ACCGGACTGAGAGCTTTCGGCTTCACCCTGAGAC |
| | | | AGTGCTACCAGCAAGTGGGCTGCCTGCTGCTGTT |
| | | | TATCGCCATGGGCATCTTCACCTTCTCTGCCGCCG |
| | | | TGTACAGCGTGGAACACGATGTGCCTAGCACCAA |
| | | | CTTCACCACCATTCCTCACTCTTGGTGGTGGGCCG |
| | | | CTGTGTCTATCTCTACAGTCGGCTACGGCGACAT |
| | | | GTACCCAGAGACACACCTGGGCAGATTCTTCGCC |
| | | | TTCCTGTGTATCGCCTTCGGCATCATCCTGAACGG |
| | | | CATGCCCATCAGCATCCTGTACAACAAGTTCAGC |
| | | | GACTACTACAGCAAGCTCAAGGCCTACGAGTACA |
| | | | CCACAATTCGGAGAGAGCGGGGCGAAGTCAACT |
| | | | TCATGCAGCGGGCCAGAAAGAAAATCGCCGAGT |
| | | | GCCTGCTGGGCAGCAATCCTCAGCTGACCCCTCG |
| | | | GCAAGAGAACTGACGATTTCTGGATCCACGCTAG |
| | | | CAATCAACCTCTGGATTACAAAATTTGTGAAAGA |
| | | | TTGACTGGTATTCTTAACTATGTTGCTCCTTTTAC |
| | | | GCTATGTGGATACGCTGCTTTAATGCCTTTGTATC |
| | | | ATGCTATTGCTTCCCGTATGGCTTTCATTTTCTCC |
| | | | TCCTTGTATAAATCCTGGTTGCTGTCTCTTTATGA |
| | | | GGAGTTGTGGCCCGTTGTCAGGCAACGTGGCGTG |
| | | | GTGTGCACTGTGTTTGCTGACGCAACCCCCACTG |
| | | | GTTGGGGCATTGCCACCACCTGTCAGCTCCTTTCC |
| | | | GGGACTTTCGCTTTCCCCCTCCCTATTGCCACGGC |
| | | | GGAACTCATCGCCGCCTGCCTTGCCCGCTGCTGG |
| | | | ACAGGGGCTCGGCTGTTGGGCACTGACAATTCCG |
| | | | TGGTGTTGTCGGGGAAATCATCGTCCTTTCCTTGG |
| | | | CTGCTCGCCTGTGTTGCCACCTGGATTCTGCGCGG |
| | | | GACGTCCTTCTGCTACGTCCCTTCGGCCCTCAATC |
| | | | CAGCGGACCTTCCTTCCCGCGGCCTGCTGCCGGC |
| | | | TCTGCGGCCTCTTCCGCGTCTTCGCCTTCGCCCTC |
| | | | AGACGAGTCGGATCTCCCTTTGGGCCGCCTCCCC |
| | | | GCACCCAGCTTTCTTGTACAAAGTGGGAATTCCT |
| | | | AGAGCTCGCTGATCAGCCTCGACTGTGCCTTCTA |
| | | | GTTGCCAGCCATCTGTTGTTTGCCCCTCCCCCGTG |
| | | | CCTTCCTTGACCCTGGAAGGTGCCACTCCCACTGT |
| | | | CCTTTCCTAATAAAATGAGGAAATTGCATCGCAT |
| | | | TGTCTGAGTAGGTGTCATTCTATTCTGGGGGGTG |
| | | | GGGTGGGGCAGGACAGCAAGGGGGAGGATTGGG |
| | | | AAGAGAATAGCAGGCATGCTGGGGAGGGCCGCA |
| | | | GGAACCCCTAGTGATGGAGTTGGCCACTCCCTCT |
| | | | CTGCGCGCTCGCTCGCTCACTGAGGCCGGGCGAC |
| | | | CAAAGGTCGCCCGACGCCCGGGCTTTGCCCGGGC |
| | | | GGCCTCAGTGAGCGAGCGAGCGCGCAGCTGCCTG |
| | | | CAGGGGCGCCTGATGCGGTATTTTCTCCTTACGC |
| | | | ATCTGTGCGGTATTTCACACCGCATACGTCAAAG |
| | | | CAACCATAGTACGCGCCCTGTAGCGGCGCATTAA |
| | | | GCGCGGCGGGTGTGGTGGTTACGCGCAGCGTGAC |
| | | | CGCTACACTTGCCAGCGCCTTAGCGCCCGCTCCTT |
| | | | TCGCTTTCTTCCCTTCCTTTCTCGCCACGTTCGCC |
| | | | GGCTTTCCCCGTCAAGCTCTAAATCGGGGGCTCC |
| | | | CTTTAGGGTTCCGATTTAGTGCTTTACGGCACCTC |
| | | | GACCCCAAAAAACTTGATTTGGGTGATGGTTCAC |
| | | | GTAGTGGGCCATCGCCCTGATAGACGGTTTTTCG |
| | | | CCCTTTGACGTTGGAGTCCACGTTCTTTAATAGTG |
| | | | GACTCTTGTTCCAAACTGGAACAACACTCAACTC |
| | | | TATCTCGGGCTATTCTTTTGATTTATAAGGGATTT |
| | | | TGCCGATTTCGGTCTATTGGTTAAAAAATGAGCT |
| | | | GATTTAACAAAAATTTAACGCGAATTTTAACAAA |
| | | | ATATTAACGTTTACAATTTTATGGTGCACTCTCAG |
| | | | TACAATCTGCTCTGATGCCGCATAGTTAAGCCAG |
| | | | CCCCGACACCCGCCAACACCCGCTGACGCGCCCT |
| | | | GACGGGCTTGTCTGCTCCCGGCATCCGCTTACAG |
| | | | ACAAGCTGTGACCGTCTCCGGGAGCTGCATGTGT |
| | | | CAGAGGTTTTCACCGTCATCACCGAAACGCGCGA |
| | | | GACGAAAGGGCCTCGTGATACGCCTATTTTTATA |
| | | | GGTTAATGTCATGATAATAATGGTTTCTTAGACG |
| | | | TCCTGGCCCGTGTCTCAAAATCTCTGATGTTACAT |
| | | | TGCACAAGATAAAAATATATCATCATGAACAATA |
| | | | AAACTGTCTGCTTACATAAACAGTAATACAAGGG |
| | | | GTGTTATGAGCCATATTCAACGGGAAACGTCGAG |
| | | | GCCGCGATTAAATTCCAACATGGATGCTGATTTA |
| | | | TATGGGTATAAATGGGCTCGCGATAATGTCGGGC |
| | | | AATCAGGTGCGACAATCTATCGCTTGTATGGGAA |
| | | | GCCCGATGCGCCAGAGTTGTTTCTGAAACATGGC |
| | | | AAAGGTAGCGTTGCCAATGATGTTACAGATGAGA |
| | | | TGGTCAGACTAAACTGGCTGACGGAATTTATGCC |
| | | | TCTTCCGACCATCAAGCATTTTATCCGTACTCCTG |
| | | | ATGATGCATGGTTACTCACCACTGCGATCCCCGG |
| | | | AAAAACAGCATTCCAGGTATTAGAAGAATATCCT |
| | | | GATTCAGGTGAAAATATTGTTGATGCGCTGGCAG |
| | | | TGTTCCTGCGCCGGTTGCATTCGATTCCTGTTTGT |
| | | | AATTGTCCTTTTAACAGCGATCGCGTATTTCGTCT |
| | | | CGCTCAGGCGCAATCACGAATGAATAACGGTTTG |
| | | | GTTGATGCGAGTGATTTTGATGACGAGCGTAATG |
| | | | GCTGGCCTGTTGAACAAGTCTGGAAAGAAATGCA |
| | | | TAAACTTTTGCCATTCTCACCGGATTCAGTCGTCA |
| | | | CTCATGGTGATTTCTCACTTGATAACCTTATTTTT |
| | | | GACGAGGGGAAATTAATAGGTTGTATTGATGTTG |
| | | | GACGAGTCGGAATCGCAGACCGATACCAGGATCT |
| | | | TGCCATCCTATGGAACTGCCTCGGTGAGTTTTCTC |
| | | | CTTCATTACAGAAACGGCTTTTTCAAAAATATGG |
| | | | TATTGATAATCCTGATATGAATAAATTGCAGTTTC |
| | | | ATTTGATGCTCGATGAGTTTTTCTAATCAGAATTG |
| | | | GTTAATTGGTTGTAACACTGGCAGAGCATTACGC |
| | | | TGACTTGACGGGACGGCGCAAGCTCATGACCAAA |
| | | | ATCCCTTAACGTGAGTTACGCGTGAAGATCCTTTT |
| | | | TGATAATCTCATGACCAAAATCCCTTAACGTGAG |
| | | | TTTTCGTTCCACTGAGCGTCAGACCCCGTAGAAA |
| | | | AGATCAAAGGATCTTCTTGAGATCCTTTTTTTCTG |
| | | | CGCGTAATCTGCTGCTTGCAAACAAAAAAACCAC |
| | | | CGCTACCAGCGGTGGTTTGTTTGCCGGATCAAGA |
| | | | GCTACCAACTCTTTTTCCGAAGGTAACTGGCTTCA |
| | | | GCAGAGCGCAGATACCAAATACTGTTCTTCTAGT |
| | | | GTAGCCGTAGTTAGGCCACCACTTCAAGAACTCT |
| | | | GTAGCACCGCCTACATACCTCGCTCTGCTAATCCT |
| | | | GTTACCAGTGGCTGCTGCCAGTGGCGATAAGTCG |
| | | | TGTCTTACCGGGTTGGACTCAAGACGATAGTTAC |
| | | | CGGATAAGGCGCAGCGGTCGGGCTGAACGGGGG |
| | | | GTTCGTGCACACAGCCCAGCTTGGAGCGAACGAC |
| | | | CTACACCGAACTGAGATACCTACAGCGTGAGCTA |
| | | | TGAGAAAGCGCCACGCTTCCCGAAGGGAGAAAG |
| | | | GCGGACAGGTATCCGGTAAGCGGCAGGGTCGGA |
| | | | ACAGGAGAGCGCACGAGGGAGCTTCCAGGGGGA |
| | | | AACGCCTGGTATCTTTATAGTCCTGTCGGGTTTCG |
| | | | CCACCTCTGACTTGAGCGTCGATTTTTGTGATGCT |
| | | | CGTCAGGGGGGCGGAGCCTATGGAAAAACGCCA |
| | | | GCAACGCGGCCTTTTTACGGTTCCTGGCCTTTTGC |
| | | | TGGCCTTTTGCTCACATGT |
| 2 | AAVss- | AAV2 5′ | CCTGCAGGCAGCTGCGCGCTCGCTCGCTCACTGA |
| | CAG- | ITR: | GGCCGCCCGGGCAAAGCCCGGGCGTCGGGCGAC |
| | KCNV2- | 1-141 bp | CTTTGGTCGCCCGGCCTCAGTGAGCGAGCGAGCG |
| | WT | CAG: | CGCAGAGAGGGAGTGGCCAACTCCATCACTAGG |
| | | 169-1901 bp | GGTTCCTTCTAGACAACTTTGTATAGAAAAGTTG |
| | | Kozak: | CTCGACATTGATTATTGACTAGTTATTAATAGTAA |
| | | 1926-1931 bp | TCAATTACGGGGTCATTAGTTCATAGCCCATATA |
| | | KCNV2 | TGGAGTTCCGCGTTACATAACTTACGGTAAATGG |
| | | (WT) | CCCGCCTGGCTGACCGCCCAACGACCCCCGCCCA |
| | | 1932-3569 bp | TTGACGTCAATAATGACGTATGTTCCCATAGTAA |
| | | WPREmut6: | CGCCAATAGGGACTTTCCATTGACGTCAATGGGT |
| | | 3570-4180 bp | GGAGTATTTACGGTAAACTGCCCACTTGGCAGTA |
| | | BGH pA: | CATCAAGTGTATCATATGCCAAGTACGCCCCCTA |
| | | 4235-4442 bp | TTGACGTCAATGACGGTAAATGGCCCGCCTGGCA |
| | | AAV2 3′ | TTATGCCCAGTACATGACCTTATGGGACTTTCCTA |
| | | ITR: | CTTGGCAGTACATCTACGTATTAGTCATCGCTATT |
| | | 4450-4590 bp | ACCATGGTCGAGGTGAGCCCCACGTTCTGCTTCA |
| | | | CTCTCCCCATCTCCCCCCCCTCCCCACCCCCAATT |
| | | | TTGTATTTATTTATTTTTTAATTATTTTGTGCAGCG |
| | | | ATGGGGGCGGGGGGGGGGGGGGGGCGCGCGCCA |
| | | | GGCGGGGCGGGGCGGGGCGAGGGGCGGGGCGGG |
| | | | GCGAGGCGGAGAGGTGCGGCGGCAGCCAATCAG |
| | | | AGCGGCGCGCTCCGAAAGTTTCCTTTTATGGCGA |
| | | | GGCGGCGGCGGCGGCGGCCCTATAAAAAGCGAA |
| | | | GCGCGCGGCGGGCGGGAGTCGCTGCGCGCTGCCT |
| | | | TCGCCCCGTGCCCCGCTCCGCCGCCGCCTCGCGC |
| | | | CGCCCGCCCCGGCTCTGACTGACCGCGTTACTCC |
| | | | CACAGGTGAGCGGGCGGGACGGCCCTTCTCCTCC |
| | | | GGGCTGTAATTAGCGCTTGGTTTAATGACGGCTT |
| | | | GTTTCTTTTCTGTGGCTGCGTGAAAGCCTTGAGGG |
| | | | GCTCCGGGAGGGCCCTTTGTGCGGGGGGAGCGGC |
| | | | TCGGGGGGTGCGTGCGTGTGTGTGTGCGTGGGGA |
| | | | GCGCCGCGTGCGGCTCCGCGCTGCCCGGCGGCTG |
| | | | TGAGCGCTGCGGGCGCGGCGCGGGGCTTTGTGCG |
| | | | CTCCGCAGTGTGCGCGAGGGGAGCGCGGCCGGG |
| | | | GGCGGTGCCCCGCGGTGCGGGGGGGGCTGCGAG |
| | | | GGGAACAAAGGCTGCGTGCGGGGTGTGTGCGTG |
| | | | GGGGGGTGAGCAGGGGGTGTGGGCGCGTCGGTC |
| | | | GGGCTGCAACCCCCCCTGCACCCCCCTCCCCGAG |
| | | | TTGCTGAGCACGGCCCGGCTTCGGGTGCGGGGCT |
| | | | CCGTACGGGGCGTGGCGCGGGGCTCGCCGTGCCG |
| | | | GGCGGGGGGTGGCGGCAGGTGGGGGTGCCGGGC |
| | | | GGGGCGGGGCCGCCTCGGGCCGGGGAGGGCTCG |
| | | | GGGGAGGGGCGCGGCGGCCCCCGGAGCGCCGGC |
| | | | GGCTGTCGAGGCGCGGCGAGCCGCAGCCATTGCC |
| | | | TTTTATGGTAATCGTGCGAGAGGGCGCAGGGACT |
| | | | TCCTTTGTCCCAAATCTGTGCGGAGCCGAAATCT |
| | | | GGGAGGCGCCGCCGCACCCCCTCTAGCGGGCGCG |
| | | | GGGCGAAGCGGTGCGGCGCCGGCAGGAAGGAAA |
| | | | TGGGCGGGGAGGGCCTTCGTGCGTCGCCGCGCCG |
| | | | CCGTCCCCTTCTCCCTCTCCAGCCTCGGGGCTGTC |
| | | | CGCGGGGGGACGGCTGCCTTCGGGGGGGACGGG |
| | | | GCAGGGCGGGGTTCGGCTTCTGGCGTGTGACCGG |
| | | | CGGCTCTAGAGCCTCTGCTAACCATGTTCATGCCT |
| | | | TCTTCTTTTTCCTACAGCTCCTGGGCAACGTGCTG |
| | | | GTTATTGTGCTGTCTCATCATTTTGGCAAAGAATT |
| | | | GCAAGTTTGTACAAAAAAGCAGGCTGCCACCATG |
| | | | CTCAAACAGAGTGAGAGGAGACGGTCCTGGAGC |
| | | | TACAGGCCCTGGAACACGACGGAGAATGAGGGC |
| | | | AGCCAACACCGCAGGAGCATTTGCTCCCTGGGTG |
| | | | CCCGTTCCGGCTCCCAGGCCAGCATCCACGGCTG |
| | | | GACAGAGGGCAACTATAACTACTACATCGAGGA |
| | | | AGACGAAGACGGCGAGGAGGAGGACCAGTGGAA |
| | | | GGACGACCTGGCAGAAGAGGACCAGCAGGCAGG |
| | | | GGAGGTCACCACCGCCAAGCCCGAGGGCCCCAG |
| | | | CGACCCTCCGGCCCTGCTGTCCACGCTGAATGTG |
| | | | AACGTGGGTGGCCACAGCTACCAGCTGGACTACT |
| | | | GCGAGCTGGCCGGCTTCCCCAAGACGCGCCTAGG |
| | | | TCGCCTGGCCACCTCCACCAGCCGCAGCCGCCAG |
| | | | CTAAGCCTGTGCGACGACTACGAGGAGCAGACA |
| | | | GACGAATACTTCTTCGACCGCGACCCGGCCGTCT |
| | | | TCCAGCTGGTCTACAATTTCTACCTGTCCGGGGTG |
| | | | CTGCTGGTGCTCGACGGGCTGTGTCCGCGCCGCT |
| | | | TCCTGGAGGAGCTGGGCTACTGGGGCGTGCGGCT |
| | | | CAAGTACACGCCACGCTGCTGCCGCATCTGCTTC |
| | | | GAGGAGCGGCGCGACGAGCTGAGCGAACGGCTC |
| | | | AAGATCCAGCACGAGCTGCGCGCGCAGGCGCAG |
| | | | GTCGAGGAGGCGGAGGAACTCTTCCGCGACATGC |
| | | | GCTTCTACGGCCCGCAGCGGCGCCGCCTCTGGAA |
| | | | CCTCATGGAGAAGCCATTCTCCTCGGTGGCCGCC |
| | | | AAGGCCATCGGGGTGGCCTCCAGCACCTTCGTGC |
| | | | TCGTCTCCGTGGTGGCGCTGGCGCTCAACACCGT |
| | | | GGAGGAGATGCAGCAGCACTCGGGGCAGGGCGA |
| | | | GGGCGGCCCAGACCTGCGGCCCATCCTGGAGCAC |
| | | | GTGGAGATGCTGTGCATGGGCTTCTTCACGCTCG |
| | | | AGTACCTGCTGCGCCTAGCCTCCACGCCCGACCT |
| | | | GAGGCGCTTCGCGCGCAGCGCCCTCAACCTGGTG |
| | | | GACCTGGTGGCCATCCTGCCGCTCTACCTTCAGCT |
| | | | GCTGCTCGAGTGCTTCACGGGCGAGGGCCACCAA |
| | | | CGCGGCCAGACGGTGGGCAGCGTGGGTAAGGTG |
| | | | GGTCAGGTGTTGCGCGTCATGCGCCTCATGCGCA |
| | | | TCTTCCGCATCCTCAAGCTGGCGCGCCACTCCAC |
| | | | CGGACTGCGTGCCTTCGGCTTCACGCTGCGCCAG |
| | | | TGCTACCAGCAGGTGGGCTGCCTGCTGCTCTTCA |
| | | | TCGCCATGGGCATCTTCACTTTCTCTGCGGCTGTC |
| | | | TACTCTGTGGAGCACGATGTGCCCAGCACCAACT |
| | | | TCACTACCATCCCCCACTCCTGGTGGTGGGCCGC |
| | | | GGTGAGCATCTCCACCGTGGGCTACGGAGACATG |
| | | | TACCCAGAGACCCACCTGGGCAGGTTTTTTGCCT |
| | | | TCCTCTGCATTGCTTTTGGGATCATTCTCAACGGG |
| | | | ATGCCCATTTCCATCCTCTACAACAAGTTTTCTGA |
| | | | TTACTACAGCAAGCTGAAGGCTTATGAGTATACC |
| | | | ACCATACGCAGGGAGAGGGGAGAGGTGAACTTC |
| | | | ATGCAGAGAGCCAGAAAGAAGATAGCTGAGTGT |
| | | | TTGCTTGGAAGCAACCCACAGCTCACCCCAAGAC |
| | | | AAGAGAATTAGCGATTTCTGGATCCACGCTAGCA |
| | | | ATCAACCTCTGGATTACAAAATTTGTGAAAGATT |
| | | | GACTGGTATTCTTAACTATGTTGCTCCTTTTACGC |
| | | | TATGTGGATACGCTGCTTTAATGCCTTTGTATCAT |
| | | | GCTATTGCTTCCCGTATGGCTTTCATTTTCTCCTC |
| | | | CTTGTATAAATCCTGGTTGCTGTCTCTTTATGAGG |
| | | | AGTTGTGGCCCGTTGTCAGGCAACGTGGCGTGGT |
| | | | GTGCACTGTGTTTGCTGACGCAACCCCCACTGGT |
| | | | TGGGGCATTGCCACCACCTGTCAGCTCCTTTCCG |
| | | | GGACTTTCGCTTTCCCCCTCCCTATTGCCACGGCG |
| | | | GAACTCATCGCCGCCTGCCTTGCCCGCTGCTGGA |
| | | | CAGGGGCTCGGCTGTTGGGCACTGACAATTCCGT |
| | | | GGTGTTGTCGGGGAAATCATCGTCCTTTCCTTGGC |
| | | | TGCTCGCCTGTGTTGCCACCTGGATTCTGCGCGG |
| | | | GACGTCCTTCTGCTACGTCCCTTCGGCCCTCAATC |
| | | | CAGCGGACCTTCCTTCCCGCGGCCTGCTGCCGGC |
| | | | TCTGCGGCCTCTTCCGCGTCTTCGCCTTCGCCCTC |
| | | | AGACGAGTCGGATCTCCCTTTGGGCCGCCTCCCC |
| | | | GCACCCAGCTTTCTTGTACAAAGTGGGAATTCCT |
| | | | AGAGCTCGCTGATCAGCCTCGACTGTGCCTTCTA |
| | | | GTTGCCAGCCATCTGTTGTTTGCCCCTCCCCCGTG |
| | | | CCTTCCTTGACCCTGGAAGGTGCCACTCCCACTGT |
| | | | CCTTTCCTAATAAAATGAGGAAATTGCATCGCAT |
| | | | TGTCTGAGTAGGTGTCATTCTATTCTGGGGGGTG |
| | | | GGGTGGGGCAGGACAGCAAGGGGGAGGATTGGG |
| | | | AAGAGAATAGCAGGCATGCTGGGGAGGGCCGCA |
| | | | GGAACCCCTAGTGATGGAGTTGGCCACTCCCTCT |
| | | | CTGCGCGCTCGCTCGCTCACTGAGGCCGGGCGAC |
| | | | CAAAGGTCGCCCGACGCCCGGGCTTTGCCCGGGC |
| | | | GGCCTCAGTGAGCGAGCGAGCGCGCAGCTGCCTG |
| | | | CAGGGGCGCCTGATGCGGTATTTTCTCCTTACGC |
| | | | ATCTGTGCGGTATTTCACACCGCATACGTCAAAG |
| | | | CAACCATAGTACGCGCCCTGTAGCGGCGCATTAA |
| | | | GCGCGGCGGGTGTGGTGGTTACGCGCAGCGTGAC |
| | | | CGCTACACTTGCCAGCGCCTTAGCGCCCGCTCCTT |
| | | | TCGCTTTCTTCCCTTCCTTTCTCGCCACGTTCGCC |
| | | | GGCTTTCCCCGTCAAGCTCTAAATCGGGGGCTCC |
| | | | CTTTAGGGTTCCGATTTAGTGCTTTACGGCACCTC |
| | | | GACCCCAAAAAACTTGATTTGGGTGATGGTTCAC |
| | | | GTAGTGGGCCATCGCCCTGATAGACGGTTTTTCG |
| | | | CCCTTTGACGTTGGAGTCCACGTTCTTTAATAGTG |
| | | | GACTCTTGTTCCAAACTGGAACAACACTCAACTC |
| | | | TATCTCGGGCTATTCTTTTGATTTATAAGGGATTT |
| | | | TGCCGATTTCGGTCTATTGGTTAAAAAATGAGCT |
| | | | GATTTAACAAAAATTTAACGCGAATTTTAACAAA |
| | | | ATATTAACGTTTACAATTTTATGGTGCACTCTCAG |
| | | | TACAATCTGCTCTGATGCCGCATAGTTAAGCCAG |
| | | | CCCCGACACCCGCCAACACCCGCTGACGCGCCCT |
| | | | GACGGGCTTGTCTGCTCCCGGCATCCGCTTACAG |
| | | | ACAAGCTGTGACCGTCTCCGGGAGCTGCATGTGT |
| | | | CAGAGGTTTTCACCGTCATCACCGAAACGCGCGA |
| | | | GACGAAAGGGCCTCGTGATACGCCTATTTTTATA |
| | | | GGTTAATGTCATGATAATAATGGTTTCTTAGACG |
| | | | TCCTGGCCCGTGTCTCAAAATCTCTGATGTTACAT |
| | | | TGCACAAGATAAAAATATATCATCATGAACAATA |
| | | | AAACTGTCTGCTTACATAAACAGTAATACAAGGG |
| | | | GTGTTATGAGCCATATTCAACGGGAAACGTCGAG |
| | | | GCCGCGATTAAATTCCAACATGGATGCTGATTTA |
| | | | TATGGGTATAAATGGGCTCGCGATAATGTCGGGC |
| | | | AATCAGGTGCGACAATCTATCGCTTGTATGGGAA |
| | | | GCCCGATGCGCCAGAGTTGTTTCTGAAACATGGC |
| | | | AAAGGTAGCGTTGCCAATGATGTTACAGATGAGA |
| | | | TGGTCAGACTAAACTGGCTGACGGAATTTATGCC |
| | | | TCTTCCGACCATCAAGCATTTTATCCGTACTCCTG |
| | | | ATGATGCATGGTTACTCACCACTGCGATCCCCGG |
| | | | AAAAACAGCATTCCAGGTATTAGAAGAATATCCT |
| | | | GATTCAGGTGAAAATATTGTTGATGCGCTGGCAG |
| | | | TGTTCCTGCGCCGGTTGCATTCGATTCCTGTTTGT |
| | | | AATTGTCCTTTTAACAGCGATCGCGTATTTCGTCT |
| | | | CGCTCAGGCGCAATCACGAATGAATAACGGTTTG |
| | | | GTTGATGCGAGTGATTTTGATGACGAGCGTAATG |
| | | | GCTGGCCTGTTGAACAAGTCTGGAAAGAAATGCA |
| | | | TAAACTTTTGCCATTCTCACCGGATTCAGTCGTCA |
| | | | CTCATGGTGATTTCTCACTTGATAACCTTATTTTT |
| | | | GACGAGGGGAAATTAATAGGTTGTATTGATGTTG |
| | | | GACGAGTCGGAATCGCAGACCGATACCAGGATCT |
| | | | TGCCATCCTATGGAACTGCCTCGGTGAGTTTTCTC |
| | | | CTTCATTACAGAAACGGCTTTTTCAAAAATATGG |
| | | | TATTGATAATCCTGATATGAATAAATTGCAGTTTC |
| | | | ATTTGATGCTCGATGAGTTTTTCTAATCAGAATTG |
| | | | GTTAATTGGTTGTAACACTGGCAGAGCATTACGC |
| | | | TGACTTGACGGGACGGCGCAAGCTCATGACCAAA |
| | | | ATCCCTTAACGTGAGTTACGCGTGAAGATCCTTTT |
| | | | TGATAATCTCATGACCAAAATCCCTTAACGTGAG |
| | | | TTTTCGTTCCACTGAGCGTCAGACCCCGTAGAAA |
| | | | AGATCAAAGGATCTTCTTGAGATCCTTTTTTTCTG |
| | | | CGCGTAATCTGCTGCTTGCAAACAAAAAAACCAC |
| | | | CGCTACCAGCGGTGGTTTGTTTGCCGGATCAAGA |
| | | | GCTACCAACTCTTTTTCCGAAGGTAACTGGCTTCA |
| | | | GCAGAGCGCAGATACCAAATACTGTTCTTCTAGT |
| | | | GTAGCCGTAGTTAGGCCACCACTTCAAGAACTCT |
| | | | GTAGCACCGCCTACATACCTCGCTCTGCTAATCCT |
| | | | GTTACCAGTGGCTGCTGCCAGTGGCGATAAGTCG |
| | | | TGTCTTACCGGGTTGGACTCAAGACGATAGTTAC |
| | | | CGGATAAGGCGCAGCGGTCGGGCTGAACGGGGG |
| | | | GTTCGTGCACACAGCCCAGCTTGGAGCGAACGAC |
| | | | CTACACCGAACTGAGATACCTACAGCGTGAGCTA |
| | | | TGAGAAAGCGCCACGCTTCCCGAAGGGAGAAAG |
| | | | GCGGACAGGTATCCGGTAAGCGGCAGGGTCGGA |
| | | | ACAGGAGAGCGCACGAGGGAGCTTCCAGGGGGA |
| | | | AACGCCTGGTATCTTTATAGTCCTGTCGGGTTTCG |
| | | | CCACCTCTGACTTGAGCGTCGATTTTTGTGATGCT |
| | | | CGTCAGGGGGGCGGAGCCTATGGAAAAACGCCA |
| | | | GCAACGCGGCCTTTTTACGGTTCCTGGCCTTTTGC |
| | | | TGGCCTTTTGCTCACATGT |
| 3 | AAVss- | AAV2 5′ | CCTGCAGGCAGCTGCGCGCTCGCTCGCTCACTGA |
| | RK- | ITR: | GGCCGCCCGGGCAAAGCCCGGGCGTCGGGCGAC |
| | KCNV2- | 1-141 bp | CTTTGGTCGCCCGGCCTCAGTGAGCGAGCGAGCG |
| | OPTI | RK: | CGCAGAGAGGGAGTGGCCAACTCCATCACTAGG |
| | | 169-788 bp | GGTTCCTTCTAGACAACTTTGTATAGAAAAGTTG |
| | | Kozak: | TGTAGTTAATGATTAACCCGCCATGCTACTTATCT |
| | | 813-818 bp | ACGTACATTTATATTGGCTCATGTCCAACATTACC |
| | | KCNV2(Opti): | GCCATGTTGACATTGATTATTGACTAGAATTCGCT |
| | | 819-2456 bp | AGCAAGATCCAAGCTCAGATCTCGATCGAGTTGG |
| | | WPRE(mut6): | GCCCCAGAAGCCTGGTGGTTGTTTGTCCTTCTCAG |
| | | 2457-3067 bp | GGGAAAAGTGAGGCGGCCCCTTGGAGGAAGGGG |
| | | BGH pA: | CCGGGCAGAATGATCTAATCGGATTCCAAGCAGC |
| | | 3122-3329 bp | TCAGGGGATTGTCTTTTTCTAGCACCTTCTTGCCA |
| | | AAV2 3′ | CTCCTAAGCGTCCTCCGTGACCCCGGCTGGGATT |
| | | ITR: | TAGCCTGGTGCTGTGTCAGCCCCGGTCTCCCAGG |
| | | 3337-3477 bp | GGCTTCCCAGTGGTCCCCAGGAACCCTCGACAGG |
| | | | GCCCGGTCTCTCTCGTCCAGCAAGGGCAGGGACG |
| | | | GGCCACAGGCCAAGGGCCCTCGATCGAGGAACT |
| | | | GAAAAACCAGAAAGTTAACTGGTAAGTTTAGTCT |
| | | | TTTTGTCTTTTATTTCAGGTCCCGGATCCGGTGGT |
| | | | GGTGCAAATCAAAGAACTGCTCCTCAGTGGATGT |
| | | | TGCCTTTACTTCTAGGCCTGTACGGAAGTGTTACT |
| | | | TCTGCTCTAAAAGCTGCGGAATTGTACCCGCGGC |
| | | | CGCCAAGTTTGTACAAAAAAGCAGGCTGCCACCA |
| | | | TGCTGAAGCAGAGCGAGAGAAGGCGGAGCTGGT |
| | | | CCTACAGACCTTGGAACACCACAGAGAACGAGG |
| | | | GCAGCCAGCACAGAAGATCCATCTGTTCTCTGGG |
| | | | CGCCAGAAGCGGCTCTCAGGCCTCTATTCATGGC |
| | | | TGGACCGAGGGCAACTACAACTACTACATCGAAG |
| | | | AGGACGAGGACGGCGAGGAAGAGGACCAGTGGA |
| | | | AAGATGACCTGGCCGAGGAAGATCAGCAGGCCG |
| | | | GCGAAGTGACAACAGCCAAGCCTGAAGGACCTA |
| | | | GCGATCCTCCTGCTCTGCTGAGCACCCTGAATGT |
| | | | GAATGTCGGCGGCCACAGCTACCAGCTGGATTAC |
| | | | TGTGAACTGGCCGGCTTTCCCAAGACCAGACTGG |
| | | | GCAGACTGGCCACCAGCACAAGCAGATCTAGAC |
| | | | AGCTGAGCCTGTGCGACGACTACGAGGAACAGA |
| | | | CCGACGAGTACTTCTTCGACAGAGATCCCGCCGT |
| | | | GTTTCAGCTGGTGTACAACTTCTACCTGAGCGGC |
| | | | GTGCTGCTGGTGCTGGATGGACTGTGCCCTCGGA |
| | | | GATTTCTGGAAGAACTCGGCTACTGGGGCGTCAG |
| | | | ACTGAAGTACACCCCTCGGTGCTGCCGGATCTGC |
| | | | TTCGAGGAAAGAAGGGACGAGCTGAGCGAGCGG |
| | | | CTGAAGATCCAGCATGAACTGAGAGCCCAGGCTC |
| | | | AGGTGGAAGAGGCCGAAGAACTGTTCCGGGACA |
| | | | TGAGATTCTACGGCCCTCAACGGCGGAGACTGTG |
| | | | GAACCTGATGGAAAAGCCTTTTAGCAGCGTGGCC |
| | | | GCCAAGGCCATTGGAGTGGCCTCTTCTACATTCG |
| | | | TGCTGGTGTCTGTGGTGGCCCTGGCTCTGAATAC |
| | | | CGTGGAAGAGATGCAGCAGCACTCTGGCCAAGG |
| | | | CGAAGGCGGACCTGATCTGAGGCCTATCCTGGAA |
| | | | CACGTGGAAATGCTGTGCATGGGCTTTTTCACCC |
| | | | TGGAATACCTGCTGCGGCTGGCCTCTACACCCGA |
| | | | CCTGAGAAGATTTGCCAGATCTGCCCTGAACCTG |
| | | | GTGGATCTGGTGGCTATCCTGCCTCTGTATCTGCA |
| | | | GCTGCTGCTGGAATGTTTTACCGGCGAGGGACAT |
| | | | CAGAGGGGCCAGACAGTGGGATCTGTGGGCAAA |
| | | | GTTGGACAGGTGCTGAGAGTGATGCGGCTGATGA |
| | | | GAATCTTCCGGATCCTGAAGCTGGCCAGACACAG |
| | | | CACCGGACTGAGAGCTTTCGGCTTCACCCTGAGA |
| | | | CAGTGCTACCAGCAAGTGGGCTGCCTGCTGCTGT |
| | | | TTATCGCCATGGGCATCTTCACCTTCTCTGCCGCC |
| | | | GTGTACAGCGTGGAACACGATGTGCCTAGCACCA |
| | | | ACTTCACCACCATTCCTCACTCTTGGTGGTGGGCC |
| | | | GCTGTGTCTATCTCTACAGTCGGCTACGGCGACA |
| | | | TGTACCCAGAGACACACCTGGGCAGATTCTTCGC |
| | | | CTTCCTGTGTATCGCCTTCGGCATCATCCTGAACG |
| | | | GCATGCCCATCAGCATCCTGTACAACAAGTTCAG |
| | | | CGACTACTACAGCAAGCTCAAGGCCTACGAGTAC |
| | | | ACCACAATTCGGAGAGAGCGGGGCGAAGTCAAC |
| | | | TTCATGCAGCGGGCCAGAAAGAAAATCGCCGAG |
| | | | TGCCTGCTGGGCAGCAATCCTCAGCTGACCCCTC |
| | | | GGCAAGAGAACTGACGATTTCTGGATCCACGCTA |
| | | | GCAATCAACCTCTGGATTACAAAATTTGTGAAAG |
| | | | ATTGACTGGTATTCTTAACTATGTTGCTCCTTTTA |
| | | | CGCTATGTGGATACGCTGCTTTAATGCCTTTGTAT |
| | | | CATGCTATTGCTTCCCGTATGGCTTTCATTTTCTC |
| | | | CTCCTTGTATAAATCCTGGTTGCTGTCTCTTTATG |
| | | | AGGAGTTGTGGCCCGTTGTCAGGCAACGTGGCGT |
| | | | GGTGTGCACTGTGTTTGCTGACGCAACCCCCACT |
| | | | GGTTGGGGCATTGCCACCACCTGTCAGCTCCTTTC |
| | | | CGGGACTTTCGCTTTCCCCCTCCCTATTGCCACGG |
| | | | CGGAACTCATCGCCGCCTGCCTTGCCCGCTGCTG |
| | | | GACAGGGGCTCGGCTGTTGGGCACTGACAATTCC |
| | | | GTGGTGTTGTCGGGGAAATCATCGTCCTTTCCTTG |
| | | | GCTGCTCGCCTGTGTTGCCACCTGGATTCTGCGCG |
| | | | GGACGTCCTTCTGCTACGTCCCTTCGGCCCTCAAT |
| | | | CCAGCGGACCTTCCTTCCCGCGGCCTGCTGCCGG |
| | | | CTCTGCGGCCTCTTCCGCGTCTTCGCCTTCGCCCT |
| | | | CAGACGAGTCGGATCTCCCTTTGGGCCGCCTCCC |
| | | | CGCACCCAGCTTTCTTGTACAAAGTGGGAATTCC |
| | | | TAGAGCTCGCTGATCAGCCTCGACTGTGCCTTCT |
| | | | AGTTGCCAGCCATCTGTTGTTTGCCCCTCCCCCGT |
| | | | GCCTTCCTTGACCCTGGAAGGTGCCACTCCCACT |
| | | | GTCCTTTCCTAATAAAATGAGGAAATTGCATCGC |
| | | | ATTGTCTGAGTAGGTGTCATTCTATTCTGGGGGGT |
| | | | GGGGTGGGGCAGGACAGCAAGGGGGAGGATTGG |
| | | | GAAGAGAATAGCAGGCATGCTGGGGAGGGCCGC |
| | | | AGGAACCCCTAGTGATGGAGTTGGCCACTCCCTC |
| | | | TCTGCGCGCTCGCTCGCTCACTGAGGCCGGGCGA |
| | | | CCAAAGGTCGCCCGACGCCCGGGCTTTGCCCGGG |
| | | | CGGCCTCAGTGAGCGAGCGAGCGCGCAGCTGCCT |
| | | | GCAGGGGCGCCTGATGCGGTATTTTCTCCTTACG |
| | | | CATCTGTGCGGTATTTCACACCGCATACGTCAAA |
| | | | GCAACCATAGTACGCGCCCTGTAGCGGCGCATTA |
| | | | AGCGCGGCGGGTGTGGTGGTTACGCGCAGCGTGA |
| | | | CCGCTACACTTGCCAGCGCCTTAGCGCCCGCTCC |
| | | | TTTCGCTTTCTTCCCTTCCTTTCTCGCCACGTTCGC |
| | | | CGGCTTTCCCCGTCAAGCTCTAAATCGGGGGCTC |
| | | | CCTTTAGGGTTCCGATTTAGTGCTTTACGGCACCT |
| | | | CGACCCCAAAAAACTTGATTTGGGTGATGGTTCA |
| | | | CGTAGTGGGCCATCGCCCTGATAGACGGTTTTTC |
| | | | GCCCTTTGACGTTGGAGTCCACGTTCTTTAATAGT |
| | | | GGACTCTTGTTCCAAACTGGAACAACACTCAACT |
| | | | CTATCTCGGGCTATTCTTTTGATTTATAAGGGATT |
| | | | TTGCCGATTTCGGTCTATTGGTTAAAAAATGAGC |
| | | | TGATTTAACAAAAATTTAACGCGAATTTTAACAA |
| | | | AATATTAACGTTTACAATTTTATGGTGCACTCTCA |
| | | | GTACAATCTGCTCTGATGCCGCATAGTTAAGCCA |
| | | | GCCCCGACACCCGCCAACACCCGCTGACGCGCCC |
| | | | TGACGGGCTTGTCTGCTCCCGGCATCCGCTTACA |
| | | | GACAAGCTGTGACCGTCTCCGGGAGCTGCATGTG |
| | | | TCAGAGGTTTTCACCGTCATCACCGAAACGCGCG |
| | | | AGACGAAAGGGCCTCGTGATACGCCTATTTTTAT |
| | | | AGGTTAATGTCATGATAATAATGGTTTCTTAGAC |
| | | | GTCCTGGCCCGTGTCTCAAAATCTCTGATGTTACA |
| | | | TTGCACAAGATAAAAATATATCATCATGAACAAT |
| | | | AAAACTGTCTGCTTACATAAACAGTAATACAAGG |
| | | | GGTGTTATGAGCCATATTCAACGGGAAACGTCGA |
| | | | GGCCGCGATTAAATTCCAACATGGATGCTGATTT |
| | | | ATATGGGTATAAATGGGCTCGCGATAATGTCGGG |
| | | | CAATCAGGTGCGACAATCTATCGCTTGTATGGGA |
| | | | AGCCCGATGCGCCAGAGTTGTTTCTGAAACATGG |
| | | | CAAAGGTAGCGTTGCCAATGATGTTACAGATGAG |
| | | | ATGGTCAGACTAAACTGGCTGACGGAATTTATGC |
| | | | CTCTTCCGACCATCAAGCATTTTATCCGTACTCCT |
| | | | GATGATGCATGGTTACTCACCACTGCGATCCCCG |
| | | | GAAAAACAGCATTCCAGGTATTAGAAGAATATCC |
| | | | TGATTCAGGTGAAAATATTGTTGATGCGCTGGCA |
| | | | GTGTTCCTGCGCCGGTTGCATTCGATTCCTGTTTG |
| | | | TAATTGTCCTTTTAACAGCGATCGCGTATTTCGTC |
| | | | TCGCTCAGGCGCAATCACGAATGAATAACGGTTT |
| | | | GGTTGATGCGAGTGATTTTGATGACGAGCGTAAT |
| | | | GGCTGGCCTGTTGAACAAGTCTGGAAAGAAATGC |
| | | | ATAAACTTTTGCCATTCTCACCGGATTCAGTCGTC |
| | | | ACTCATGGTGATTTCTCACTTGATAACCTTATTTT |
| | | | TGACGAGGGGAAATTAATAGGTTGTATTGATGTT |
| | | | GGACGAGTCGGAATCGCAGACCGATACCAGGAT |
| | | | CTTGCCATCCTATGGAACTGCCTCGGTGAGTTTTC |
| | | | TCCTTCATTACAGAAACGGCTTTTTCAAAAATAT |
| | | | GGTATTGATAATCCTGATATGAATAAATTGCAGT |
| | | | TTCATTTGATGCTCGATGAGTTTTTCTAATCAGAA |
| | | | TTGGTTAATTGGTTGTAACACTGGCAGAGCATTA |
| | | | CGCTGACTTGACGGGACGGCGCAAGCTCATGACC |
| | | | AAAATCCCTTAACGTGAGTTACGCGTGAAGATCC |
| | | | TTTTTGATAATCTCATGACCAAAATCCCTTAACGT |
| | | | GAGTTTTCGTTCCACTGAGCGTCAGACCCCGTAG |
| | | | AAAAGATCAAAGGATCTTCTTGAGATCCTTTTTTT |
| | | | CTGCGCGTAATCTGCTGCTTGCAAACAAAAAAAC |
| | | | CACCGCTACCAGCGGTGGTTTGTTTGCCGGATCA |
| | | | AGAGCTACCAACTCTTTTTCCGAAGGTAACTGGC |
| | | | TTCAGCAGAGCGCAGATACCAAATACTGTTCTTC |
| | | | TAGTGTAGCCGTAGTTAGGCCACCACTTCAAGAA |
| | | | CTCTGTAGCACCGCCTACATACCTCGCTCTGCTAA |
| | | | TCCTGTTACCAGTGGCTGCTGCCAGTGGCGATAA |
| | | | GTCGTGTCTTACCGGGTTGGACTCAAGACGATAG |
| | | | TTACCGGATAAGGCGCAGCGGTCGGGCTGAACG |
| | | | GGGGGTTCGTGCACACAGCCCAGCTTGGAGCGAA |
| | | | CGACCTACACCGAACTGAGATACCTACAGCGTGA |
| | | | GCTATGAGAAAGCGCCACGCTTCCCGAAGGGAG |
| | | | AAAGGCGGACAGGTATCCGGTAAGCGGCAGGGT |
| | | | CGGAACAGGAGAGCGCACGAGGGAGCTTCCAGG |
| | | | GGGAAACGCCTGGTATCTTTATAGTCCTGTCGGG |
| | | | TTTCGCCACCTCTGACTTGAGCGTCGATTTTTGTG |
| | | | ATGCTCGTCAGGGGGGCGGAGCCTATGGAAAAA |
| | | | CGCCAGCAACGCGGCCTTTTTACGGTTCCTGGCC |
| | | | TTTTGCTGGCCTTTTGCTCACATGT |
| 4 | AAVss- | AAV2 5′ | CCTGCAGGCAGCTGCGCGCTCGCTCGCTCACTGA |
| | RK- | ITR: | GGCCGCCCGGGCAAAGCCCGGGCGTCGGGCGAC |
| | KCNV2- | 1-141 bp | CTTTGGTCGCCCGGCCTCAGTGAGCGAGCGAGCG |
| | WT | RK: | CGCAGAGAGGGAGTGGCCAACTCCATCACTAGG |
| | | 169-788 bp | GGTTCCTTCTAGACAACTTTGTATAGAAAAGTTG |
| | | Kozak: | TGTAGTTAATGATTAACCCGCCATGCTACTTATCT |
| | | 813-818 bp | ACGTACATTTATATTGGCTCATGTCCAACATTACC |
| | | KCNV2(WT): | GCCATGTTGACATTGATTATTGACTAGAATTCGCT |
| | | 819-2456 bp | AGCAAGATCCAAGCTCAGATCTCGATCGAGTTGG |
| | | WPRE(mut6): | GCCCCAGAAGCCTGGTGGTTGTTTGTCCTTCTCAG |
| | | 2457-3067 bp | GGGAAAAGTGAGGCGGCCCCTTGGAGGAAGGGG |
| | | BGH pA: | CCGGGCAGAATGATCTAATCGGATTCCAAGCAGC |
| | | 3122-3329 bp | TCAGGGGATTGTCTTTTTCTAGCACCTTCTTGCCA |
| | | AAV2 3′ | CTCCTAAGCGTCCTCCGTGACCCCGGCTGGGATT |
| | | ITR: | TAGCCTGGTGCTGTGTCAGCCCCGGTCTCCCAGG |
| | | 3337-3477 bp | GGCTTCCCAGTGGTCCCCAGGAACCCTCGACAGG |
| | | | GCCCGGTCTCTCTCGTCCAGCAAGGGCAGGGACG |
| | | | GGCCACAGGCCAAGGGCCCTCGATCGAGGAACT |
| | | | GAAAAACCAGAAAGTTAACTGGTAAGTTTAGTCT |
| | | | TTTTGTCTTTTATTTCAGGTCCCGGATCCGGTGGT |
| | | | GGTGCAAATCAAAGAACTGCTCCTCAGTGGATGT |
| | | | TGCCTTTACTTCTAGGCCTGTACGGAAGTGTTACT |
| | | | TCTGCTCTAAAAGCTGCGGAATTGTACCCGCGGC |
| | | | CGCCAAGTTTGTACAAAAAAGCAGGCTGCCACCA |
| | | | TGCTCAAACAGAGTGAGAGGAGACGGTCCTGGA |
| | | | GCTACAGGCCCTGGAACACGACGGAGAATGAGG |
| | | | GCAGCCAACACCGCAGGAGCATTTGCTCCCTGGG |
| | | | TGCCCGTTCCGGCTCCCAGGCCAGCATCCACGGC |
| | | | TGGACAGAGGGCAACTATAACTACTACATCGAGG |
| | | | AAGACGAAGACGGCGAGGAGGAGGACCAGTGGA |
| | | | AGGACGACCTGGCAGAAGAGGACCAGCAGGCAG |
| | | | GGGAGGTCACCACCGCCAAGCCCGAGGGCCCCA |
| | | | GCGACCCTCCGGCCCTGCTGTCCACGCTGAATGT |
| | | | GAACGTGGGTGGCCACAGCTACCAGCTGGACTAC |
| | | | TGCGAGCTGGCCGGCTTCCCCAAGACGCGCCTAG |
| | | | GTCGCCTGGCCACCTCCACCAGCCGCAGCCGCCA |
| | | | GCTAAGCCTGTGCGACGACTACGAGGAGCAGAC |
| | | | AGACGAATACTTCTTCGACCGCGACCCGGCCGTC |
| | | | TTCCAGCTGGTCTACAATTTCTACCTGTCCGGGGT |
| | | | GCTGCTGGTGCTCGACGGGCTGTGTCCGCGCCGC |
| | | | TTCCTGGAGGAGCTGGGCTACTGGGGCGTGCGGC |
| | | | TCAAGTACACGCCACGCTGCTGCCGCATCTGCTT |
| | | | CGAGGAGCGGCGCGACGAGCTGAGCGAACGGCT |
| | | | CAAGATCCAGCACGAGCTGCGCGCGCAGGCGCA |
| | | | GGTCGAGGAGGCGGAGGAACTCTTCCGCGACAT |
| | | | GCGCTTCTACGGCCCGCAGCGGCGCCGCCTCTGG |
| | | | AACCTCATGGAGAAGCCATTCTCCTCGGTGGCCG |
| | | | CCAAGGCCATCGGGGTGGCCTCCAGCACCTTCGT |
| | | | GCTCGTCTCCGTGGTGGCGCTGGCGCTCAACACC |
| | | | GTGGAGGAGATGCAGCAGCACTCGGGGCAGGGC |
| | | | GAGGGCGGCCCAGACCTGCGGCCCATCCTGGAGC |
| | | | ACGTGGAGATGCTGTGCATGGGCTTCTTCACGCT |
| | | | CGAGTACCTGCTGCGCCTAGCCTCCACGCCCGAC |
| | | | CTGAGGCGCTTCGCGCGCAGCGCCCTCAACCTGG |
| | | | TGGACCTGGTGGCCATCCTGCCGCTCTACCTTCA |
| | | | GCTGCTGCTCGAGTGCTTCACGGGCGAGGGCCAC |
| | | | CAACGCGGCCAGACGGTGGGCAGCGTGGGTAAG |
| | | | GTGGGTCAGGTGTTGCGCGTCATGCGCCTCATGC |
| | | | GCATCTTCCGCATCCTCAAGCTGGCGCGCCACTC |
| | | | CACCGGACTGCGTGCCTTCGGCTTCACGCTGCGC |
| | | | CAGTGCTACCAGCAGGTGGGCTGCCTGCTGCTCT |
| | | | TCATCGCCATGGGCATCTTCACTTTCTCTGCGGCT |
| | | | GTCTACTCTGTGGAGCACGATGTGCCCAGCACCA |
| | | | ACTTCACTACCATCCCCCACTCCTGGTGGTGGGC |
| | | | CGCGGTGAGCATCTCCACCGTGGGCTACGGAGAC |
| | | | ATGTACCCAGAGACCCACCTGGGCAGGTTTTTTG |
| | | | CCTTCCTCTGCATTGCTTTTGGGATCATTCTCAAC |
| | | | GGGATGCCCATTTCCATCCTCTACAACAAGTTTTC |
| | | | TGATTACTACAGCAAGCTGAAGGCTTATGAGTAT |
| | | | ACCACCATACGCAGGGAGAGGGGAGAGGTGAAC |
| | | | TTCATGCAGAGAGCCAGAAAGAAGATAGCTGAG |
| | | | TGTTTGCTTGGAAGCAACCCACAGCTCACCCCAA |
| | | | GACAAGAGAATTAGCGATTTCTGGATCCACGCTA |
| | | | GCAATCAACCTCTGGATTACAAAATTTGTGAAAG |
| | | | ATTGACTGGTATTCTTAACTATGTTGCTCCTTTTA |
| | | | CGCTATGTGGATACGCTGCTTTAATGCCTTTGTAT |
| | | | CATGCTATTGCTTCCCGTATGGCTTTCATTTTCTC |
| | | | CTCCTTGTATAAATCCTGGTTGCTGTCTCTTTATG |
| | | | AGGAGTTGTGGCCCGTTGTCAGGCAACGTGGCGT |
| | | | GGTGTGCACTGTGTTTGCTGACGCAACCCCCACT |
| | | | GGTTGGGGCATTGCCACCACCTGTCAGCTCCTTTC |
| | | | CGGGACTTTCGCTTTCCCCCTCCCTATTGCCACGG |
| | | | CGGAACTCATCGCCGCCTGCCTTGCCCGCTGCTG |
| | | | GACAGGGGCTCGGCTGTTGGGCACTGACAATTCC |
| | | | GTGGTGTTGTCGGGGAAATCATCGTCCTTTCCTTG |
| | | | GCTGCTCGCCTGTGTTGCCACCTGGATTCTGCGCG |
| | | | GGACGTCCTTCTGCTACGTCCCTTCGGCCCTCAAT |
| | | | CCAGCGGACCTTCCTTCCCGCGGCCTGCTGCCGG |
| | | | CTCTGCGGCCTCTTCCGCGTCTTCGCCTTCGCCCT |
| | | | CAGACGAGTCGGATCTCCCTTTGGGCCGCCTCCC |
| | | | CGCACCCAGCTTTCTTGTACAAAGTGGGAATTCC |
| | | | TAGAGCTCGCTGATCAGCCTCGACTGTGCCTTCT |
| | | | AGTTGCCAGCCATCTGTTGTTTGCCCCTCCCCCGT |
| | | | GCCTTCCTTGACCCTGGAAGGTGCCACTCCCACT |
| | | | GTCCTTTCCTAATAAAATGAGGAAATTGCATCGC |
| | | | ATTGTCTGAGTAGGTGTCATTCTATTCTGGGGGGT |
| | | | GGGGTGGGGCAGGACAGCAAGGGGGAGGATTGG |
| | | | GAAGAGAATAGCAGGCATGCTGGGGAGGGCCGC |
| | | | AGGAACCCCTAGTGATGGAGTTGGCCACTCCCTC |
| | | | TCTGCGCGCTCGCTCGCTCACTGAGGCCGGGCGA |
| | | | CCAAAGGTCGCCCGACGCCCGGGCTTTGCCCGGG |
| | | | CGGCCTCAGTGAGCGAGCGAGCGCGCAGCTGCCT |
| | | | GCAGGGGCGCCTGATGCGGTATTTTCTCCTTACG |
| | | | CATCTGTGCGGTATTTCACACCGCATACGTCAAA |
| | | | GCAACCATAGTACGCGCCCTGTAGCGGCGCATTA |
| | | | AGCGCGGCGGGTGTGGTGGTTACGCGCAGCGTGA |
| | | | CCGCTACACTTGCCAGCGCCTTAGCGCCCGCTCC |
| | | | TTTCGCTTTCTTCCCTTCCTTTCTCGCCACGTTCGC |
| | | | CGGCTTTCCCCGTCAAGCTCTAAATCGGGGGCTC |
| | | | CCTTTAGGGTTCCGATTTAGTGCTTTACGGCACCT |
| | | | CGACCCCAAAAAACTTGATTTGGGTGATGGTTCA |
| | | | CGTAGTGGGCCATCGCCCTGATAGACGGTTTTTC |
| | | | GCCCTTTGACGTTGGAGTCCACGTTCTTTAATAGT |
| | | | GGACTCTTGTTCCAAACTGGAACAACACTCAACT |
| | | | CTATCTCGGGCTATTCTTTTGATTTATAAGGGATT |
| | | | TTGCCGATTTCGGTCTATTGGTTAAAAAATGAGC |
| | | | TGATTTAACAAAAATTTAACGCGAATTTTAACAA |
| | | | AATATTAACGTTTACAATTTTATGGTGCACTCTCA |
| | | | GTACAATCTGCTCTGATGCCGCATAGTTAAGCCA |
| | | | GCCCCGACACCCGCCAACACCCGCTGACGCGCCC |
| | | | TGACGGGCTTGTCTGCTCCCGGCATCCGCTTACA |
| | | | GACAAGCTGTGACCGTCTCCGGGAGCTGCATGTG |
| | | | TCAGAGGTTTTCACCGTCATCACCGAAACGCGCG |
| | | | AGACGAAAGGGCCTCGTGATACGCCTATTTTTAT |
| | | | AGGTTAATGTCATGATAATAATGGTTTCTTAGAC |
| | | | GTCCTGGCCCGTGTCTCAAAATCTCTGATGTTACA |
| | | | TTGCACAAGATAAAAATATATCATCATGAACAAT |
| | | | AAAACTGTCTGCTTACATAAACAGTAATACAAGG |
| | | | GGTGTTATGAGCCATATTCAACGGGAAACGTCGA |
| | | | GGCCGCGATTAAATTCCAACATGGATGCTGATTT |
| | | | ATATGGGTATAAATGGGCTCGCGATAATGTCGGG |
| | | | CAATCAGGTGCGACAATCTATCGCTTGTATGGGA |
| | | | AGCCCGATGCGCCAGAGTTGTTTCTGAAACATGG |
| | | | CAAAGGTAGCGTTGCCAATGATGTTACAGATGAG |
| | | | ATGGTCAGACTAAACTGGCTGACGGAATTTATGC |
| | | | CTCTTCCGACCATCAAGCATTTTATCCGTACTCCT |
| | | | GATGATGCATGGTTACTCACCACTGCGATCCCCG |
| | | | GAAAAACAGCATTCCAGGTATTAGAAGAATATCC |
| | | | TGATTCAGGTGAAAATATTGTTGATGCGCTGGCA |
| | | | GTGTTCCTGCGCCGGTTGCATTCGATTCCTGTTTG |
| | | | TAATTGTCCTTTTAACAGCGATCGCGTATTTCGTC |
| | | | TCGCTCAGGCGCAATCACGAATGAATAACGGTTT |
| | | | GGTTGATGCGAGTGATTTTGATGACGAGCGTAAT |
| | | | GGCTGGCCTGTTGAACAAGTCTGGAAAGAAATGC |
| | | | ATAAACTTTTGCCATTCTCACCGGATTCAGTCGTC |
| | | | ACTCATGGTGATTTCTCACTTGATAACCTTATTTT |
| | | | TGACGAGGGGAAATTAATAGGTTGTATTGATGTT |
| | | | GGACGAGTCGGAATCGCAGACCGATACCAGGAT |
| | | | CTTGCCATCCTATGGAACTGCCTCGGTGAGTTTTC |
| | | | TCCTTCATTACAGAAACGGCTTTTTCAAAAATAT |
| | | | GGTATTGATAATCCTGATATGAATAAATTGCAGT |
| | | | TTCATTTGATGCTCGATGAGTTTTTCTAATCAGAA |
| | | | TTGGTTAATTGGTTGTAACACTGGCAGAGCATTA |
| | | | CGCTGACTTGACGGGACGGCGCAAGCTCATGACC |
| | | | AAAATCCCTTAACGTGAGTTACGCGTGAAGATCC |
| | | | TTTTTGATAATCTCATGACCAAAATCCCTTAACGT |
| | | | GAGTTTTCGTTCCACTGAGCGTCAGACCCCGTAG |
| | | | AAAAGATCAAAGGATCTTCTTGAGATCCTTTTTTT |
| | | | CTGCGCGTAATCTGCTGCTTGCAAACAAAAAAAC |
| | | | CACCGCTACCAGCGGTGGTTTGTTTGCCGGATCA |
| | | | AGAGCTACCAACTCTTTTTCCGAAGGTAACTGGC |
| | | | TTCAGCAGAGCGCAGATACCAAATACTGTTCTTC |
| | | | TAGTGTAGCCGTAGTTAGGCCACCACTTCAAGAA |
| | | | CTCTGTAGCACCGCCTACATACCTCGCTCTGCTAA |
| | | | TCCTGTTACCAGTGGCTGCTGCCAGTGGCGATAA |
| | | | GTCGTGTCTTACCGGGTTGGACTCAAGACGATAG |
| | | | TTACCGGATAAGGCGCAGCGGTCGGGCTGAACG |
| | | | GGGGGTTCGTGCACACAGCCCAGCTTGGAGCGAA |
| | | | CGACCTACACCGAACTGAGATACCTACAGCGTGA |
| | | | GCTATGAGAAAGCGCCACGCTTCCCGAAGGGAG |
| | | | AAAGGCGGACAGGTATCCGGTAAGCGGCAGGGT |
| | | | CGGAACAGGAGAGCGCACGAGGGAGCTTCCAGG |
| | | | GGGAAACGCCTGGTATCTTTATAGTCCTGTCGGG |
| | | | TTTCGCCACCTCTGACTTGAGCGTCGATTTTTGTG |
| | | | ATGCTCGTCAGGGGGGCGGAGCCTATGGAAAAA |
| | | | CGCCAGCAACGCGGCCTTTTTACGGTTCCTGGCC |
| | | | TTTTGCTGGCCTTTTGCTCACATGT |
| 5 | AAV2 | 141 bp | CCTGCAGGCAGCTGCGCGCTCGCTCGCTCACTGA |
| | 5′ ITR | | GGCCGCCCGGGCAAAGCCCGGGCGTCGGGCGAC |
| | | | CTTTGGTCGCCCGGCCTCAGTGAGCGAGCGAGCG |
| | | | CGCAGAGAGGGAGTGGCCAACTCCATCACTAGG |
| | | | GGTTCCT |
| 6 | AAV2 | 141 bp | AGGAACCCCTAGTGATGGAGTTGGCCACTCCCTC |
| | 3′ ITR | | TCTGCGCGCTCGCTCGCTCACTGAGGCCGGGCGA |
| | | | CCAAAGGTCGCCCGACGCCCGGGCTTTGCCCGGG |
| | | | CGGCCTCAGTGAGCGAGCGAGCGCGCAGCTGCCT |
| | | | GCAGG |
| 7 | RK | | TGTAGTTAATGATTAACCCGCCATGCTACTTATCT |
| | promoter | | ACGTACATTTATATTGGCTCATGTCCAACATTACC |
| | | | GCCATGTTGACATTGATTATTGACTAGAATTCGCT |
| | | | AGCAAGATCCAAGCTCAGATCTCGATCGAGTTGG |
| | | | GCCCCAGAAGCCTGGTGGTTGTTTGTCCTTCTCAG |
| | | | GGGAAAAGTGAGGCGGCCCCTTGGAGGAAGGGG |
| | | | CCGGGCAGAATGATCTAATCGGATTCCAAGCAGC |
| | | | TCAGGGGATTGTCTTTTTCTAGCACCTTCTTGCCA |
| | | | CTCCTAAGCGTCCTCCGTGACCCCGGCTGGGATT |
| | | | TAGCCTGGTGCTGTGTCAGCCCCGGTCTCCCAGG |
| | | | GGCTTCCCAGTGGTCCCCAGGAACCCTCGACAGG |
| | | | GCCCGGTCTCTCTCGTCCAGCAAGGGCAGGGACG |
| | | | GGCCACAGGCCAAGGGCCCTCGATCGAGGAACT |
| | | | GAAAAAC |
| 8 | CAG | | CTCGACATTGATTATTGACTAGTTATTAATAGTAA |
| | promoter | | TCAATTACGGGGTCATTAGTTCATAGCCCATATA |
| | | | TGGAGTTCCGCGTTACATAACTTACGGTAAATGG |
| | | | CCCGCCTGGCTGACCGCCCAACGACCCCCGCCCA |
| | | | TTGACGTCAATAATGACGTATGTTCCCATAGTAA |
| | | | CGCCAATAGGGACTTTCCATTGACGTCAATGGGT |
| | | | GGAGTATTTACGGTAAACTGCCCACTTGGCAGTA |
| | | | CATCAAGTGTATCATATGCCAAGTACGCCCCCTA |
| | | | TTGACGTCAATGACGGTAAATGGCCCGCCTGGCA |
| | | | TTATGCCCAGTACATGACCTTATGGGACTTTCCTA |
| | | | CTTGGCAGTACATCTACGTATTAGTCATCGCTATT |
| | | | ACCATGGTCGAGGTGAGCCCCACGTTCTGCTTCA |
| | | | CTCTCCCCATCTCCCCCCCCTCCCCACCCCCAATT |
| | | | TTGTATTTATTTATTTTTTAATTATTTTGTGCAGCG |
| | | | ATGGGGGCGGGGGGGGGGGGGGGGCGCGCGCCA |
| | | | GGCGGGGCGGGGCGGGGCGAGGGGGGGGGGGG |
| | | | GCGAGGCGGAGAGGTGCGGCGGCAGCCAATCAG |
| | | | AGCGGCGCGCTCCGAAAGTTTCCTTTTATGGCGA |
| | | | GGCGGCGGCGGCGGCGGCCCTATAAAAAGCGAA |
| | | | GCGCGCGGCGGGCGGGAGTCGCTGCGCGCTGCCT |
| | | | TCGCCCCGTGCCCCGCTCCGCCGCCGCCTCGCGC |
| | | | CGCCCGCCCCGGCTCTGACTGACCGCGTTACTCC |
| | | | CACAGGTGAGCGGGCGGGACGGCCCTTCTCCTCC |
| | | | GGGCTGTAATTAGCGCTTGGTTTAATGACGGCTT |
| | | | GTTTCTTTTCTGTGGCTGCGTGAAAGCCTTGAGGG |
| | | | GCTCCGGGAGGGCCCTTTGTGCGGGGGGAGCGGC |
| | | | TCGGGGGGTGCGTGCGTGTGTGTGTGCGTGGGGA |
| | | | GCGCCGCGTGCGGCTCCGCGCTGCCCGGCGGCTG |
| | | | TGAGCGCTGCGGGCGCGGCGCGGGGCTTTGTGCG |
| | | | CTCCGCAGTGTGCGCGAGGGGAGCGCGGCCGGG |
| | | | GGCGGTGCCCCGCGGTGCGGGGGGGGCTGCGAG |
| | | | GGGAACAAAGGCTGCGTGCGGGGTGTGTGCGTG |
| | | | GGGGGGTGAGCAGGGGGTGTGGGCGCGTCGGTC |
| | | | GGGCTGCAACCCCCCCTGCACCCCCCTCCCCGAG |
| | | | TTGCTGAGCACGGCCCGGCTTCGGGTGCGGGGCT |
| | | | CCGTACGGGGCGTGGCGCGGGGCTCGCCGTGCCG |
| | | | GGCGGGGGGTGGCGGCAGGTGGGGGTGCCGGGC |
| | | | GGGGCGGGGCCGCCTCGGGCCGGGGAGGGCTCG |
| | | | GGGGAGGGGCGCGGCGGCCCCCGGAGCGCCGGC |
| | | | GGCTGTCGAGGCGCGGCGAGCCGCAGCCATTGCC |
| | | | TTTTATGGTAATCGTGCGAGAGGGCGCAGGGACT |
| | | | TCCTTTGTCCCAAATCTGTGCGGAGCCGAAATCT |
| | | | GGGAGGCGCCGCCGCACCCCCTCTAGCGGGCGCG |
| | | | GGGCGAAGCGGTGCGGCGCCGGCAGGAAGGAAA |
| | | | TGGGCGGGGAGGGCCTTCGTGCGTCGCCGCGCCG |
| | | | CCGTCCCCTTCTCCCTCTCCAGCCTCGGGGCTGTC |
| | | | CGCGGGGGGACGGCTGCCTTCGGGGGGGACGGG |
| | | | GCAGGGCGGGGTTCGGCTTCTGGCGTGTGACCGG |
| | | | CGGCTCTAGAGCCTCTGCTAACCATGTTCATGCCT |
| | | | TCTTCTTTTTCCTACAGCTCCTGGGCAACGTGCTG |
| | | | GTTATTGTGCTGTCTCATCATTTTGGCAAAGAATT |
| | | | G |
| 9 | KCNV2- | | ATGCTCAAACAGAGTGAGAGGAGACGGTCCTGG |
| | WT | | AGCTACAGGCCCTGGAACACGACGGAGAATGAG |
| | | | GGCAGCCAACACCGCAGGAGCATTTGCTCCCTGG |
| | | | GTGCCCGTTCCGGCTCCCAGGCCAGCATCCACGG |
| | | | CTGGACAGAGGGCAACTATAACTACTACATCGAG |
| | | | GAAGACGAAGACGGCGAGGAGGAGGACCAGTGG |
| | | | AAGGACGACCTGGCAGAAGAGGACCAGCAGGCA |
| | | | GGGGAGGTCACCACCGCCAAGCCCGAGGGCCCC |
| | | | AGCGACCCTCCGGCCCTGCTGTCCACGCTGAATG |
| | | | TGAACGTGGGTGGCCACAGCTACCAGCTGGACTA |
| | | | CTGCGAGCTGGCCGGCTTCCCCAAGACGCGCCTA |
| | | | GGTCGCCTGGCCACCTCCACCAGCCGCAGCCGCC |
| | | | AGCTAAGCCTGTGCGACGACTACGAGGAGCAGA |
| | | | CAGACGAATACTTCTTCGACCGCGACCCGGCCGT |
| | | | CTTCCAGCTGGTCTACAATTTCTACCTGTCCGGGG |
| | | | TGCTGCTGGTGCTCGACGGGCTGTGTCCGCGCCG |
| | | | CTTCCTGGAGGAGCTGGGCTACTGGGGCGTGCGG |
| | | | CTCAAGTACACGCCACGCTGCTGCCGCATCTGCT |
| | | | TCGAGGAGCGGCGCGACGAGCTGAGCGAACGGC |
| | | | TCAAGATCCAGCACGAGCTGCGCGCGCAGGCGC |
| | | | AGGTCGAGGAGGCGGAGGAACTCTTCCGCGACA |
| | | | TGCGCTTCTACGGCCCGCAGCGGCGCCGCCTCTG |
| | | | GAACCTCATGGAGAAGCCATTCTCCTCGGTGGCC |
| | | | GCCAAGGCCATCGGGGTGGCCTCCAGCACCTTCG |
| | | | TGCTCGTCTCCGTGGTGGCGCTGGCGCTCAACAC |
| | | | CGTGGAGGAGATGCAGCAGCACTCGGGGCAGGG |
| | | | CGAGGGCGGCCCAGACCTGCGGCCCATCCTGGAG |
| | | | CACGTGGAGATGCTGTGCATGGGCTTCTTCACGC |
| | | | TCGAGTACCTGCTGCGCCTAGCCTCCACGCCCGA |
| | | | CCTGAGGCGCTTCGCGCGCAGCGCCCTCAACCTG |
| | | | GTGGACCTGGTGGCCATCCTGCCGCTCTACCTTC |
| | | | AGCTGCTGCTCGAGTGCTTCACGGGCGAGGGCCA |
| | | | CCAACGCGGCCAGACGGTGGGCAGCGTGGGTAA |
| | | | GGTGGGTCAGGTGTTGCGCGTCATGCGCCTCATG |
| | | | CGCATCTTCCGCATCCTCAAGCTGGCGCGCCACT |
| | | | CCACCGGACTGCGTGCCTTCGGCTTCACGCTGCG |
| | | | CCAGTGCTACCAGCAGGTGGGCTGCCTGCTGCTC |
| | | | TTCATCGCCATGGGCATCTTCACTTTCTCTGCGGC |
| | | | TGTCTACTCTGTGGAGCACGATGTGCCCAGCACC |
| | | | AACTTCACTACCATCCCCCACTCCTGGTGGTGGG |
| | | | CCGCGGTGAGCATCTCCACCGTGGGCTACGGAGA |
| | | | CATGTACCCAGAGACCCACCTGGGCAGGTTTTTT |
| | | | GCCTTCCTCTGCATTGCTTTTGGGATCATTCTCAA |
| | | | CGGGATGCCCATTTCCATCCTCTACAACAAGTTTT |
| | | | CTGATTACTACAGCAAGCTGAAGGCTTATGAGTA |
| | | | TACCACCATACGCAGGGAGAGGGGAGAGGTGAA |
| | | | CTTCATGCAGAGAGCCAGAAAGAAGATAGCTGA |
| | | | GTGTTTGCTTGGAAGCAACCCACAGCTCACCCCA |
| | | | AGACAAGAGAATTAG |
| 10 | KCNV2- | | ATGCTGAAGCAGAGCGAGAGAAGGCGGAGCTGG |
| | Opti | | TCCTACAGACCTTGGAACACCACAGAGAACGAG |
| | | | GGCAGCCAGCACAGAAGATCCATCTGTTCTCTGG |
| | | | GCGCCAGAAGCGGCTCTCAGGCCTCTATTCATGG |
| | | | CTGGACCGAGGGCAACTACAACTACTACATCGAA |
| | | | GAGGACGAGGACGGCGAGGAAGAGGACCAGTGG |
| | | | AAAGATGACCTGGCCGAGGAAGATCAGCAGGCC |
| | | | GGCGAAGTGACAACAGCCAAGCCTGAAGGACCT |
| | | | AGCGATCCTCCTGCTCTGCTGAGCACCCTGAATG |
| | | | TGAATGTCGGCGGCCACAGCTACCAGCTGGATTA |
| | | | CTGTGAACTGGCCGGCTTTCCCAAGACCAGACTG |
| | | | GGCAGACTGGCCACCAGCACAAGCAGATCTAGA |
| | | | CAGCTGAGCCTGTGCGACGACTACGAGGAACAG |
| | | | ACCGACGAGTACTTCTTCGACAGAGATCCCGCCG |
| | | | TGTTTCAGCTGGTGTACAACTTCTACCTGAGCGG |
| | | | CGTGCTGCTGGTGCTGGATGGACTGTGCCCTCGG |
| | | | AGATTTCTGGAAGAACTCGGCTACTGGGGCGTCA |
| | | | GACTGAAGTACACCCCTCGGTGCTGCCGGATCTG |
| | | | CTTCGAGGAAAGAAGGGACGAGCTGAGCGAGCG |
| | | | GCTGAAGATCCAGCATGAACTGAGAGCCCAGGCT |
| | | | CAGGTGGAAGAGGCCGAAGAACTGTTCCGGGAC |
| | | | ATGAGATTCTACGGCCCTCAACGGCGGAGACTGT |
| | | | GGAACCTGATGGAAAAGCCTTTTAGCAGCGTGGC |
| | | | CGCCAAGGCCATTGGAGTGGCCTCTTCTACATTC |
| | | | GTGCTGGTGTCTGTGGTGGCCCTGGCTCTGAATA |
| | | | CCGTGGAAGAGATGCAGCAGCACTCTGGCCAAG |
| | | | GCGAAGGCGGACCTGATCTGAGGCCTATCCTGGA |
| | | | ACACGTGGAAATGCTGTGCATGGGCTTTTTCACC |
| | | | CTGGAATACCTGCTGCGGCTGGCCTCTACACCCG |
| | | | ACCTGAGAAGATTTGCCAGATCTGCCCTGAACCT |
| | | | GGTGGATCTGGTGGCTATCCTGCCTCTGTATCTGC |
| | | | AGCTGCTGCTGGAATGTTTTACCGGCGAGGGACA |
| | | | TCAGAGGGGCCAGACAGTGGGATCTGTGGGCAA |
| | | | AGTTGGACAGGTGCTGAGAGTGATGCGGCTGATG |
| | | | AGAATCTTCCGGATCCTGAAGCTGGCCAGACACA |
| | | | GCACCGGACTGAGAGCTTTCGGCTTCACCCTGAG |
| | | | ACAGTGCTACCAGCAAGTGGGCTGCCTGCTGCTG |
| | | | TTTATCGCCATGGGCATCTTCACCTTCTCTGCCGC |
| | | | CGTGTACAGCGTGGAACACGATGTGCCTAGCACC |
| | | | AACTTCACCACCATTCCTCACTCTTGGTGGTGGGC |
| | | | CGCTGTGTCTATCTCTACAGTCGGCTACGGCGAC |
| | | | ATGTACCCAGAGACACACCTGGGCAGATTCTTCG |
| | | | CCTTCCTGTGTATCGCCTTCGGCATCATCCTGAAC |
| | | | GGCATGCCCATCAGCATCCTGTACAACAAGTTCA |
| | | | GCGACTACTACAGCAAGCTCAAGGCCTACGAGTA |
| | | | CACCACAATTCGGAGAGAGCGGGGCGAAGTCAA |
| | | | CTTCATGCAGCGGGCCAGAAAGAAAATCGCCGA |
| | | | GTGCCTGCTGGGCAGCAATCCTCAGCTGACCCCT |
| | | | CGGCAAGAGAACTGA |
| 11 | WPRE | | AATCAACCTCTGGATTACAAAATTTGTGAAAGAT |
| | (mut6) | | TGACTGGTATTCTTAACTATGTTGCTCCTTTTACG |
| | | | CTATGTGGATACGCTGCTTTAATGCCTTTGTATCA |
| | | | TGCTATTGCTTCCCGTATGGCTTTCATTTTCTCCTC |
| | | | CTTGTATAAATCCTGGTTGCTGTCTCTTTATGAGG |
| | | | AGTTGTGGCCCGTTGTCAGGCAACGTGGCGTGGT |
| | | | GTGCACTGTGTTTGCTGACGCAACCCCCACTGGT |
| | | | TGGGGCATTGCCACCACCTGTCAGCTCCTTTCCG |
| | | | GGACTTTCGCTTTCCCCCTCCCTATTGCCACGGCG |
| | | | GAACTCATCGCCGCCTGCCTTGCCCGCTGCTGGA |
| | | | CAGGGGCTCGGCTGTTGGGCACTGACAATTCCGT |
| | | | GGTGTTGTCGGGGAAATCATCGTCCTTTCCTTGGC |
| | | | TGCTCGCCTGTGTTGCCACCTGGATTCTGCGCGG |
| | | | GACGTCCTTCTGCTACGTCCCTTCGGCCCTCAATC |
| | | | CAGCGGACCTTCCTTCCCGCGGCCTGCTGCCGGC |
| | | | TCTGCGGCCTCTTCCGCGTCTTCGCCTTCGCCCTC |
| | | | AGACGAGTCGGATCTCCCTTTGGGCCGCCTCCCC |
| | | | GC |
| 12 | BGH | | CTGTGCCTTCTAGTTGCCAGCCATCTGTTGTTTGC |
| | pA | | CCCTCCCCCGTGCCTTCCTTGACCCTGGAAGGTGC |
| | | | CACTCCCACTGTCCTTTCCTAATAAAATGAGGAA |
| | | | ATTGCATCGCATTGTCTGAGTAGGTGTCATTCTAT |
| | | | TCTGGGGGGTGGGGTGGGGCAGGACAGCAAGGG |
| | | | GGAGGATTGGGAAGAGAATAGCAGGCATGCTGG |
| | | | GGA |
| 13 | Kv8.2 | | MLKQSERRRSWSYRPWNTTENEGSQHRRSICSLGA |
| | | | RSGSQASIHGWTEGNYNYYIEEDEDGEEEDQWKDD |
| | | | LAEEDQQAGEVTTAKPEGPSDPPALLSTLNVNVGG |
| | | | HSYQLDYCELAGFPKTRLGRLATSTSRSRQLSLCDD |
| | | | YEEQTDEYFFDRDPAVFQLVYNFYLSGVLLVLDGL |
| | | | CPRRFLEELGYWGVRLKYTPRCCRICFEERRDELSE |
| | | | RLKIQHELRAQAQVEEAEELFRDMRFYGPQRRRLW |
| | | | NLMEKPFSSVAAKAIGVASSTFVLVSVVALALNTV |
| | | | EEMQQHSGQGEGGPDLRPILEHVEMLCMGFFTLEY |
| | | | LLRLASTPDLRRFARSALNLVDLVAILPLYLQLLLE |
| | | | CFTGEGHQRGQTVGSVGKVGQVLRVMRLMRIFRIL |
| | | | KLARHSTGLRAFGFTLRQCYQQVGCLLLFIAMGIFT |
| | | | FSAAVYSVEHDVPSTNFTTIPHSWWWAAVSISTVG |
| | | | YGDMYPETHLGRFFAFLCIAFGIILNGMPISILYNKF |
| | | | SDYYSKLKAYEYTTIRRERGEVNFMQRARKKIAEC |
| | | | LLGSNPQLTPRQEN |

1. An expression construct comprising:
(a) a promotor sequence that confers expression in photoreceptor cells; and
(b) a nucleic acid sequence encoding Kv8.2, wherein the nucleic acid sequence comprises a sequence that is at least 90% identical to SEQ ID NO: 10,
wherein the nucleic acid sequence is operably linked to the promotor.

2. The expression construct of claim 1, wherein the promotor sequence is a CAG or rhodopsin kinase (RK) promotor sequence.

3. The expression construct of claim 2, wherein the promotor sequence comprises a sequence that is at least 90% identical to SEQ ID: NO:8.

4. The expression construct of claim 3, wherein the promotor sequence comprises a sequence of SEQ ID: NO:8.

5. The expression construct of claim 2, wherein the promotor sequence comprises a sequence that is at least 90% identical to SEQ ID: NO:7.

6. The expression construct of claim 5, wherein the promotor sequence comprises a sequence of SEQ ID: NO:7.

7. The expression construct of claim 1, wherein the expression construct further comprises a post transcriptional regulatory element.

8. The expression construct of claim 7, wherein the expression construct further comprises a woodchuck hepatitis virus post transcriptional regulatory element (WPRE).

9. The expression construct of claim 8, wherein the WPRE comprises a sequence that is at least 90% identical to SEQ ID NO:11.

10. The expression construct of claim 9, wherein the WPRE comprises a sequence comprising SEQ ID NO:11.

11.-15. (canceled)

16. The expression construct of claim 1, wherein the nucleic acid sequence encoding the Kv8.2 comprises a sequence comprising SEQ ID NO:10.

17. The expression construct of claim 1, wherein the nucleic acid sequence encoding the Kv8.2 encodes a protein comprising a sequence that is at least 90% identical to SEQ ID NO:13.

18. The expression construct of claim 17, wherein the nucleic acid sequence encoding the Kv8.2 encodes a protein comprising SEQ ID NO: 13.

19. The expression construct of claim 1, wherein the expression construct further comprises bovine growth hormone polyadenylation (BGH-polyA) signal.

20. The expression construct of claim 19, wherein the polyadenylation signal comprises a sequence that is at least 90% identical to SEQ ID NO:12.

21. The expression construct of claim 20, wherein the polyadenylation signal comprises SEQ ID NO:12.

22. The expression construct of claim 1, wherein the expression construct comprises a sequence that is at least 90% identical to a sequence selected from the group consisting of SEQ ID NOS: 1-4.

23. The expression construct of claim 22, wherein the expression construct comprises a sequence selected from the group consisting of SEQ ID NOS: 1-4.

24. A vector comprising an expression construct of claim 1.

25. The vector of claim 24, wherein the vector is a viral vector.

26. The vector of claim 25, wherein the vector is an adeno-associated virus (AAV) vector.

27. The vector of claim 26, wherein the vector comprises a genome derived from AAV serotype AAV2.

28. The vector of claim 26, wherein the vector comprises a capsid derived from AAV7m8.

29. The vector of claim 26, wherein the vector comprises a capsid derived from AAV5.

30. A pharmaceutical composition comprising the vector of claim 24 and a pharmaceutically acceptable carrier.

31. A method for treating a retinal disease in a subject in need thereof, wherein the retinal disease is associated with one or more mutations in the KCNV2 gene, the method comprising administering to the subject the vector of claim 24.

32. The method of claim 31, wherein the retinal disease is cone-dystrophy with supernormal rod response (CDSSR).

33. A method of increasing expression of KCNV2 in a subject in need thereof, the method comprising administering to the subject the vector of claim 24.

34. A method of increasing Kv8.2 levels in a photoreceptor in a subject in need thereof, the method comprising administering to the subject the vector of claim 24.

35. The method of claim 31, wherein the vector or the pharmaceutical composition is administered by intraocular injection.

36. The method of claim 35, wherein the vector or the pharmaceutical composition is injected into the central retina of the subject.


SEQUENCE LISTING (13 sequences)

SEQ ID NO: 1 - DNA, 7138 bp, synthetic construct
  source 1..7138: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgctgaagcagagcgagagaaggcggagctggtcctacagaccttggaacaccacagagaacgagggcagccagcacagaagatccatctgttctctgggcgccagaagcggctctcaggcctctattcatggctggaccgagggcaactacaactactacatcgaagaggacgaggacggcgaggaagaggaccagtggaaagatgacctggccgaggaagatcagcaggccggcgaagtgacaacagccaagcctgaaggacctagcgatcctcctgctctgctgagcaccctgaatgtgaatgtcggcggccacagctaccagctggattactgtgaactggccggctttcccaagaccagactgggcagactggccaccagcacaagcagatctagacagctgagcctgtgcgacgactacgaggaacagaccgacgagtacttcttcgacagagatcccgccgtgtttcagctggtgtacaacttctacctgagcggcgtgctgctggtgctggatggactgtgccctcggagatttctggaagaactcggctactggggcgtcagactgaagtacacccctcggtgctgccggatctgcttcgaggaaagaagggacgagctgagcgagcggctgaagatccagcatgaactgagagcccaggctcaggtggaagaggccgaagaactgttccgggacatgagattctacggccctcaacggcggagactgtggaacctgatggaaaagccttttagcagcgtggccgccaaggccattggagtggcctcttctacattcgtgctggtgtctgtggtggccctggctctgaataccgtggaagagatgcagcagcactctggccaaggcgaaggcggacctgatctgaggcctatcctggaacacgtggaaatgctgtgcatgggctttttcaccctggaatacctgctgcggctggcctctacacccgacctgagaagatttgccagatctgccctgaacctggtggatctggtggctatcctgcctctgtatctgcagctgctgctggaatgttttaccggcgagggacatcagaggggccagacagtgggatctgtgggcaaagttggacaggtgctgagagtgatgcggctgatgagaatcttccggatcctgaagctggccagacacagcaccggactgagagctttcggcttcaccctgagacagtgctaccagcaagtgggctgcctgctgctgtttatcgccatgggcatcttcaccttctctgccgccgtgtacagcgtggaacacgatgtgcctagcaccaacttcaccaccattcctcactcttggtggtgggccgctgtgtctatctctacagtcggctacggcgacatgtacccagagacacacctgggcagattcttcgccttcctgtgtatcgccttcggcatcatcctgaacggcatgcccatcagcatcctgtacaacaagttcagcgactactacagcaagctcaaggcctacgagtacaccacaattcggagagagcggggcgaagtcaacttcatgcagcgggccagaaagaaaatcgccgagtgcctgctgggcagcaatcctcagctgacccctcggcaagagaactgacgatttctggatccacgctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 7138 bp, synthetic construct
  source 1..7138: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgctcaaacagagtgagaggagacggtcctggagctacaggccctggaacacgacggagaatgagggcagccaacaccgcaggagcatttgctccctgggtgcccgttccggctcccaggccagcatccacggctggacagagggcaactataactactacatcgaggaagacgaagacggcgaggaggaggaccagtggaaggacgacctggcagaagaggaccagcaggcaggggaggtcaccaccgccaagcccgagggccccagcgaccctccggccctgctgtccacgctgaatgtgaacgtgggtggccacagctaccagctggactactgcgagctggccggcttccccaagacgcgcctaggtcgcctggccacctccaccagccgcagccgccagctaagcctgtgcgacgactacgaggagcagacagacgaatacttcttcgaccgcgacccggccgtcttccagctggtctacaatttctacctgtccggggtgctgctggtgctcgacgggctgtgtccgcgccgcttcctggaggagctgggctactggggcgtgcggctcaagtacacgccacgctgctgccgcatctgcttcgaggagcggcgcgacgagctgagcgaacggctcaagatccagcacgagctgcgcgcgcaggcgcaggtcgaggaggcggaggaactcttccgcgacatgcgcttctacggcccgcagcggcgccgcctctggaacctcatggagaagccattctcctcggtggccgccaaggccatcggggtggcctccagcaccttcgtgctcgtctccgtggtggcgctggcgctcaacaccgtggaggagatgcagcagcactcggggcagggcgagggcggcccagacctgcggcccatcctggagcacgtggagatgctgtgcatgggcttcttcacgctcgagtacctgctgcgcctagcctccacgcccgacctgaggcgcttcgcgcgcagcgccctcaacctggtggacctggtggccatcctgccgctctaccttcagctgctgctcgagtgcttcacgggcgagggccaccaacgcggccagacggtgggcagcgtgggtaaggtgggtcaggtgttgcgcgtcatgcgcctcatgcgcatcttccgcatcctcaagctggcgcgccactccaccggactgcgtgccttcggcttcacgctgcgccagtgctaccagcaggtgggctgcctgctgctcttcatcgccatgggcatcttcactttctctgcggctgtctactctgtggagcacgatgtgcccagcaccaacttcactaccatcccccactcctggtggtgggccgcggtgagcatctccaccgtgggctacggagacatgtacccagagacccacctgggcaggttttttgccttcctctgcattgcttttgggatcattctcaacgggatgcccatttccatcctctacaacaagttttctgattactacagcaagctgaaggcttatgagtataccaccatacgcagggagaggggagaggtgaacttcatgcagagagccagaaagaagatagctgagtgtttgcttggaagcaacccacagctcaccccaagacaagagaattagcgatttctggatccacgctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 3 - DNA, 6025 bp, synthetic construct
  source 1..6025: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtgtagttaatgattaacccgccatgctacttatctacgtacatttatattggctcatgtccaacattaccgccatgttgacattgattattgactagaattcgctagcaagatccaagctcagatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaagtttgtacaaaaaagcaggctgccaccatgctgaagcagagcgagagaaggcggagctggtcctacagaccttggaacaccacagagaacgagggcagccagcacagaagatccatctgttctctgggcgccagaagcggctctcaggcctctattcatggctggaccgagggcaactacaactactacatcgaagaggacgaggacggcgaggaagaggaccagtggaaagatgacctggccgaggaagatcagcaggccggcgaagtgacaacagccaagcctgaaggacctagcgatcctcctgctctgctgagcaccctgaatgtgaatgtcggcggccacagctaccagctggattactgtgaactggccggctttcccaagaccagactgggcagactggccaccagcacaagcagatctagacagctgagcctgtgcgacgactacgaggaacagaccgacgagtacttcttcgacagagatcccgccgtgtttcagctggtgtacaacttctacctgagcggcgtgctgctggtgctggatggactgtgccctcggagatttctggaagaactcggctactggggcgtcagactgaagtacacccctcggtgctgccggatctgcttcgaggaaagaagggacgagctgagcgagcggctgaagatccagcatgaactgagagcccaggctcaggtggaagaggccgaagaactgttccgggacatgagattctacggccctcaacggcggagactgtggaacctgatggaaaagccttttagcagcgtggccgccaaggccattggagtggcctcttctacattcgtgctggtgtctgtggtggccctggctctgaataccgtggaagagatgcagcagcactctggccaaggcgaaggcggacctgatctgaggcctatcctggaacacgtggaaatgctgtgcatgggctttttcaccctggaatacctgctgcggctggcctctacacccgacctgagaagatttgccagatctgccctgaacctggtggatctggtggctatcctgcctctgtatctgcagctgctgctggaatgttttaccggcgagggacatcagaggggccagacagtgggatctgtgggcaaagttggacaggtgctgagagtgatgcggctgatgagaatcttccggatcctgaagctggccagacacagcaccggactgagagctttcggcttcaccctgagacagtgctaccagcaagtgggctgcctgctgctgtttatcgccatgggcatcttcaccttctctgccgccgtgtacagcgtggaacacgatgtgcctagcaccaacttcaccaccattcctcactcttggtggtgggccgctgtgtctatctctacagtcggctacggcgacatgtacccagagacacacctgggcagattcttcgccttcctgtgtatcgccttcggcatcatcctgaacggcatgcccatcagcatcctgtacaacaagttcagcgactactacagcaagctcaaggcctacgagtacaccacaattcggagagagcggggcgaagtcaacttcatgcagcgggccagaaagaaaatcgccgagtgcctgctgggcagcaatcctcagctgacccctcggcaagagaactgacgatttctggatccacgctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 4 - DNA, 6025 bp, synthetic construct
  source 1..6025: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtgtagttaatgattaacccgccatgctacttatctacgtacatttatattggctcatgtccaacattaccgccatgttgacattgattattgactagaattcgctagcaagatccaagctcagatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaagtttgtacaaaaaagcaggctgccaccatgctcaaacagagtgagaggagacggtcctggagctacaggccctggaacacgacggagaatgagggcagccaacaccgcaggagcatttgctccctgggtgcccgttccggctcccaggccagcatccacggctggacagagggcaactataactactacatcgaggaagacgaagacggcgaggaggaggaccagtggaaggacgacctggcagaagaggaccagcaggcaggggaggtcaccaccgccaagcccgagggccccagcgaccctccggccctgctgtccacgctgaatgtgaacgtgggtggccacagctaccagctggactactgcgagctggccggcttccccaagacgcgcctaggtcgcctggccacctccaccagccgcagccgccagctaagcctgtgcgacgactacgaggagcagacagacgaatacttcttcgaccgcgacccggccgtcttccagctggtctacaatttctacctgtccggggtgctgctggtgctcgacgggctgtgtccgcgccgcttcctggaggagctgggctactggggcgtgcggctcaagtacacgccacgctgctgccgcatctgcttcgaggagcggcgcgacgagctgagcgaacggctcaagatccagcacgagctgcgcgcgcaggcgcaggtcgaggaggcggaggaactcttccgcgacatgcgcttctacggcccgcagcggcgccgcctctggaacctcatggagaagccattctcctcggtggccgccaaggccatcggggtggcctccagcaccttcgtgctcgtctccgtggtggcgctggcgctcaacaccgtggaggagatgcagcagcactcggggcagggcgagggcggcccagacctgcggcccatcctggagcacgtggagatgctgtgcatgggcttcttcacgctcgagtacctgctgcgcctagcctccacgcccgacctgaggcgcttcgcgcgcagcgccctcaacctggtggacctggtggccatcctgccgctctaccttcagctgctgctcgagtgcttcacgggcgagggccaccaacgcggccagacggtgggcagcgtgggtaaggtgggtcaggtgttgcgcgtcatgcgcctcatgcgcatcttccgcatcctcaagctggcgcgccactccaccggactgcgtgccttcggcttcacgctgcgccagtgctaccagcaggtgggctgcctgctgctcttcatcgccatgggcatcttcactttctctgcggctgtctactctgtggagcacgatgtgcccagcaccaacttcactaccatcccccactcctggtggtgggccgcggtgagcatctccaccgtgggctacggagacatgtacccagagacccacctgggcaggttttttgccttcctctgcattgcttttgggatcattctcaacgggatgcccatttccatcctctacaacaagttttctgattactacagcaagctgaaggcttatgagtataccaccatacgcagggagaggggagaggtgaacttcatgcagagagccagaaagaagatagctgagtgtttgcttggaagcaacccacagctcaccccaagacaagagaattagcgatttctggatccacgctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 452 bp, synthetic construct
  source 1..452: mol_type = other DNA
tgtagttaatgattaacccgccatgctacttatctacgtacatttatattggctcatgtccaacattaccgccatgttgacattgattattgactagaattcgctagcaagatccaagctcagatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggccctcgatcgaggaactgaaaaac

SEQ ID NO: 8 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 9 - DNA, 1638 bp, synthetic construct
  source 1..1638: mol_type = other DNA
atgctcaaacagagtgagaggagacggtcctggagctacaggccctggaacacgacggagaatgagggcagccaacaccgcaggagcatttgctccctgggtgcccgttccggctcccaggccagcatccacggctggacagagggcaactataactactacatcgaggaagacgaagacggcgaggaggaggaccagtggaaggacgacctggcagaagaggaccagcaggcaggggaggtcaccaccgccaagcccgagggccccagcgaccctccggccctgctgtccacgctgaatgtgaacgtgggtggccacagctaccagctggactactgcgagctggccggcttccccaagacgcgcctaggtcgcctggccacctccaccagccgcagccgccagctaagcctgtgcgacgactacgaggagcagacagacgaatacttcttcgaccgcgacccggccgtcttccagctggtctacaatttctacctgtccggggtgctgctggtgctcgacgggctgtgtccgcgccgcttcctggaggagctgggctactggggcgtgcggctcaagtacacgccacgctgctgccgcatctgcttcgaggagcggcgcgacgagctgagcgaacggctcaagatccagcacgagctgcgcgcgcaggcgcaggtcgaggaggcggaggaactcttccgcgacatgcgcttctacggcccgcagcggcgccgcctctggaacctcatggagaagccattctcctcggtggccgccaaggccatcggggtggcctccagcaccttcgtgctcgtctccgtggtggcgctggcgctcaacaccgtggaggagatgcagcagcactcggggcagggcgagggcggcccagacctgcggcccatcctggagcacgtggagatgctgtgcatgggcttcttcacgctcgagtacctgctgcgcctagcctccacgcccgacctgaggcgcttcgcgcgcagcgccctcaacctggtggacctggtggccatcctgccgctctaccttcagctgctgctcgagtgcttcacgggcgagggccaccaacgcggccagacggtgggcagcgtgggtaaggtgggtcaggtgttgcgcgtcatgcgcctcatgcgcatcttccgcatcctcaagctggcgcgccactccaccggactgcgtgccttcggcttcacgctgcgccagtgctaccagcaggtgggctgcctgctgctcttcatcgccatgggcatcttcactttctctgcggctgtctactctgtggagcacgatgtgcccagcaccaacttcactaccatcccccactcctggtggtgggccgcggtgagcatctccaccgtgggctacggagacatgtacccagagacccacctgggcaggttttttgccttcctctgcattgcttttgggatcattctcaacgggatgcccatttccatcctctacaacaagttttctgattactacagcaagctgaaggcttatgagtataccaccatacgcagggagaggggagaggtgaacttcatgcagagagccagaaagaagatagctgagtgtttgcttggaagcaacccacagctcaccccaagacaagagaattag

SEQ ID NO: 10 - DNA, 1638 bp, synthetic construct
  source 1..1638: mol_type = other DNA
atgctgaagcagagcgagagaaggcggagctggtcctacagaccttggaacaccacagagaacgagggcagccagcacagaagatccatctgttctctgggcgccagaagcggctctcaggcctctattcatggctggaccgagggcaactacaactactacatcgaagaggacgaggacggcgaggaagaggaccagtggaaagatgacctggccgaggaagatcagcaggccggcgaagtgacaacagccaagcctgaaggacctagcgatcctcctgctctgctgagcaccctgaatgtgaatgtcggcggccacagctaccagctggattactgtgaactggccggctttcccaagaccagactgggcagactggccaccagcacaagcagatctagacagctgagcctgtgcgacgactacgaggaacagaccgacgagtacttcttcgacagagatcccgccgtgtttcagctggtgtacaacttctacctgagcggcgtgctgctggtgctggatggactgtgccctcggagatttctggaagaactcggctactggggcgtcagactgaagtacacccctcggtgctgccggatctgcttcgaggaaagaagggacgagctgagcgagcggctgaagatccagcatgaactgagagcccaggctcaggtggaagaggccgaagaactgttccgggacatgagattctacggccctcaacggcggagactgtggaacctgatggaaaagccttttagcagcgtggccgccaaggccattggagtggcctcttctacattcgtgctggtgtctgtggtggccctggctctgaataccgtggaagagatgcagcagcactctggccaaggcgaaggcggacctgatctgaggcctatcctggaacacgtggaaatgctgtgcatgggctttttcaccctggaatacctgctgcggctggcctctacacccgacctgagaagatttgccagatctgccctgaacctggtggatctggtggctatcctgcctctgtatctgcagctgctgctggaatgttttaccggcgagggacatcagaggggccagacagtgggatctgtgggcaaagttggacaggtgctgagagtgatgcggctgatgagaatcttccggatcctgaagctggccagacacagcaccggactgagagctttcggcttcaccctgagacagtgctaccagcaagtgggctgcctgctgctgtttatcgccatgggcatcttcaccttctctgccgccgtgtacagcgtggaacacgatgtgcctagcaccaacttcaccaccattcctcactcttggtggtgggccgctgtgtctatctctacagtcggctacggcgacatgtacccagagacacacctgggcagattcttcgccttcctgtgtatcgccttcggcatcatcctgaacggcatgcccatcagcatcctgtacaacaagttcagcgactactacagcaagctcaaggcctacgagtacaccacaattcggagagagcggggcgaagtcaacttcatgcagcgggccagaaagaaaatcgccgagtgcctgctgggcagcaatcctcagctgacccctcggcaagagaactga

SEQ ID NO: 11 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 12 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 13 - protein, 545 aa, synthetic construct
MLKQSERRRSWSYRPWNTTENEGSQHRRSICSLGARSGSQASIHGWTEGNYNYYIEEDEDGEEEDQWKDDLAEEDQQAGEVTTAKPEGPSDPPALLSTLNVNVGGHSYQLDYCELAGFPKTRLGRLATSTSRSRQLSLCDDYEEQTDEYFFDRDPAVFQLVYNFYLSGVLLVLDGLCPRRFLEELGYWGVRLKYTPRCCRICFEERRDELSERLKIQHELRAQAQVEEAEELFRDMRFYGPQRRRLWNLMEKPFSSVAAKAIGVASSTFVLVSVVALALNTVEEMQQHSGQGEGGPDLRPILEHVEMLCMGFFTLEYLLRLASTPDLRRFARSALNLVDLVAILPLYLQLLLECFTGEGHQRGQTVGSVGKVGQVLRVMRLMRIFRILKLARHSTGLRAFGFTLRQCYQQVGCLLLFIAMGIFTFSAAVYSVEHDVPSTNFTTIPHSWWWAAVSISTVGYGDMYPETHLGRFFAFLCIAFGIILNGMPISILYNKFSDYYSKLKAYEYTTIRRERGEVNFMQRARKKIAECLLGSNPQLTPRQEN